SEQUENCE LISTING (29 sequences)

SEQ ID NO: 1 - protein, 311 aa, synthetic construct
  REGION 1..311: note = Synthetic sequence
MFLIIFILPTTLAVIGDFNCTNSFINDYNKTIPRISEDVVDVSLGLGTYYVLNRVYLNTTLLFTGYFPKSGANFRDLALKGSIYLSTLWYKPPFLSDFNNGIFSKVKNTKLYVNNTLYSEFSTIVIGSVFVNTSYTIVVQPHNGILEITACQYTMCEYPHTVCKSKGSIRNESWHIDSSEPLCLFKKNFTYNVSADWLYFHFYQERGVFYAYYADVGMPTTFLFSLYLGTILSHYYVMPLTCNAISSNTDNETLEYWVTPLSRRQYLLNFDEHGVITNAVDCSSSFLSEIQCKTQSFAPNTGVYDLSGFTV

SEQ ID NO: 2 - protein, 672 aa, synthetic construct
  REGION 1..672: note = Synthetic sequence
CVNYDLYGITGQGIFKEVSAAYYNNWQNLLYDSNGNIIGFKDFLTNKTYTILPCYSGRVSAAFYQNSSSPALLYRNLKCSYVLNNISFISQPFYFDSYLGCVLNAVNLTSYSVSSCDLRMGSGFCIDYALPSSRASVGGISSPYRFVTFEPFNVSFVNDSVETVGGLFEIQIPTNFTIAGHEEFIQTSSPKVTIDCSAFVCSNYAACHDLLSEYGTFCDNINSILNEVNDLLDITQLQVANALMQGVTLSSNLNTNLHSDVDNIDFKSLLGCLGSQCGSSSRSPLEDLLFNKVKLSDVGFVEAYNNCTGGSEIRDLLCVQSFNGIKVLPPILSETQISGYTTAATVAAMFPPWSAAAGVPFPLNVQYRINGLGVTMDVLNKNQKLIANAFNKALLSIQNGFTATPSALAKIQSVVNANAQALNSLLQQLFNKFGAISSSLQEILSRLDPPEAQVQIDRLINGRLTALNAYVSQQLSDITLIKAGASRAIEKVNECVKSQSPRINFCGNGNHILSLVQNAPYGLLFIHFSYKPTSFKTVLVSPGLCLSGDRGIAPKQGYFIKQNDSWMFTGSSYYYPEPISDKNVVFMNSCSVNFTKAPFIYLNNSIPNLSDFEAELSLWFKNHTSIAPNLTFNSHINATFLDLYYEMNVIQESIKSLNSSFINLKEIGTYEM

SEQ ID NO: 3 - protein, 217 aa, synthetic construct
  REGION 1..217: note = Synthetic sequence
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

SEQ ID NO: 4 - protein, 217 aa, synthetic construct
  REGION 1..217: note = Synthetic sequence
QPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNK

SEQ ID NO: 5 - protein, 217 aa, synthetic construct
  REGION 1..217: note = Synthetic sequence
QPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNK

SEQ ID NO: 6 - protein, 217 aa, synthetic construct
  REGION 1..217: note = Synthetic sequence
QPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNK

SEQ ID NO: 7 - protein, 217 aa, synthetic construct
  REGION 1..217: note = Synthetic sequence
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNK

SEQ ID NO: 8 - protein, 217 aa, synthetic construct
  REGION 1..217: note = Synthetic sequence
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRVVVLSFELLHAPATVCGPKKSTNLVKNK

SEQ ID NO: 9 - protein, 217 aa, synthetic construct
  REGION 1..217: note = Synthetic sequence
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPKKSTNLVKNK

SEQ ID NO: 10 - protein, 217 aa, synthetic construct
  REGION 1..217: note = Synthetic sequence
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYQYRLFRKSNLKPFERDISTEIYQAGSTPCNGVEGFNCYSPLQSYGFQPTNGVGYQPYRVVVLSFELLHAPATVCGPKKSTNLVKNK

SEQ ID NO: 11 - protein, 217 aa, synthetic construct
  REGION 1..217: note = Synthetic sequence
QPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVKGFNCYFPLQSYGFQPTYGVGYQPYRVVVLSFELLHAPATVCGPKKSTNLVKNK

SEQ ID NO: 12 - protein, 217 aa, synthetic construct
  REGION 1..217: note = Synthetic sequence
QPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGNTPCNGVKGFNCYFPLQSYGFQPTYGVGYQPYRVVVLSFELLHAPATVCGPKKSTNLVKNK

SEQ ID NO: 13 - protein, 27 aa, synthetic construct
  REGION 1..27: note = Synthetic sequence
GYIPEAPRDGQAYVRKDGEWVLLSTFL

SEQ ID NO: 14 - protein, 1253 aa, synthetic construct
  REGION 1..1253: note = Synthetic sequence
MFLIIFILPTTLAVIGDFNCTNSFINDYNKTIPRISEDVVDVSLGLGTYYVLNRVYLNTTLLFTGYFPKSGANFRDLALKGSIYLSTLWYKPPFLSDFNNGIFSKVKNTKLYVNNTLYSEFSTIVIGSVFVNTSYTIVVQPHNGILEITACQYTMCEYPHTVCKSKGSIRNESWHIDSSEPLCLFKKNFTYNVSADWLYFHFYQERGVFYAYYADVGMPTTFLFSLYLGTILSHYYVMPLTCNAISSNTDNETLEYWVTPLSRRQYLLNFDEHGVITNAVDCSSSFLSEIQCKTQSFAPNTGVYDLSGFTVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNYDLYGITGQGIFKEVSAAYYNNWQNLLYDSNGNIIGFKDFLTNKTYTILPCYSGRVSAAFYQNSSSPALLYRNLKCSYVLNNISFISQPFYFDSYLGCVLNAVNLTSYSVSSCDLRMGSGFCIDYALPSSRASVGGISSPYRFVTFEPFNVSFVNDSVETVGGLFEIQIPTNFTIAGHEEFIQTSSPKVTIDCSAFVCSNYAACHDLLSEYGTFCDNINSILNEVNDLLDITQLQVANALMQGVTLSSNLNTNLHSDVDNIDFKSLLGCLGSQCGSSSRSPLEDLLFNKVKLSDVGFVEAYNNCTGGSEIRDLLCVQSFNGIKVLPPILSETQISGYTTAATVAAMFPPWSAAAGVPFPLNVQYRINGLGVTMDVLNKNQKLIANAFNKALLSIQNGFTATPSALAKIQSVVNANAQALNSLLQQLFNKFGAISSSLQEILSRLDPPEAQVQIDRLINGRLTALNAYVSQQLSDITLIKAGASRAIEKVNECVKSQSPRINFCGNGNHILSLVQNAPYGLLFIHFSYKPTSFKTVLVSPGLCLSGDRGIAPKQGYFIKQNDSWMFTGSSYYYPEPISDKNVVFMNSCSVNFTKAPFIYLNNSIPNLSDFEAELSLWFKNHTSIAPNLTFNSHINATFLDLYYEMNVIQESIKSLNSSFINLKEIGTYEMGSGYIPEAPRDGQAYVRKDGEWVLLSTFLGSGLNDIFEAQKIEWHEGHHHHHH

SEQ ID NO: 15 - protein, 1229 aa, synthetic construct
  REGION 1..1229: note = Synthetic sequence
MFLIIFILPTTLAVIGDFNCTNSFINDYNKTIPRISEDVVDVSLGLGTYYVLNRVYLNTTLLFTGYFPKSGANFRDLALKGSIYLSTLWYKPPFLSDFNNGIFSKVKNTKLYVNNTLYSEFSTIVIGSVFVNTSYTIVVQPHNGILEITACQYTMCEYPHTVCKSKGSIRNESWHIDSSEPLCLFKKNFTYNVSADWLYFHFYQERGVFYAYYADVGMPTTFLFSLYLGTILSHYYVMPLTCNAISSNTDNETLEYWVTPLSRRQYLLNFDEHGVITNAVDCSSSFLSEIQCKTQSFAPNTGVYDLSGFTVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNYDLYGITGQGIFKEVSAAYYNNWQNLLYDSNGNIIGFKDFLTNKTYTILPCYSGRVSAAFYQNSSSPALLYRNLKCSYVLNNISFISQPFYFDSYLGCVLNAVNLTSYSVSSCDLRMGSGFCIDYALPSSRASVGGISSPYRFVTFEPFNVSFVNDSVETVGGLFEIQIPTNFTIAGHEEFIQTSSPKVTIDCSAFVCSNYAACHDLLSEYGTFCDNINSILNEVNDLLDITQLQVANALMQGVTLSSNLNTNLHSDVDNIDFKSLLGCLGSQCGSSSRSPLEDLLFNKVKLSDVGFVEAYNNCTGGSEIRDLLCVQSFNGIKVLPPILSETQISGYTTAATVAAMFPPWSAAAGVPFPLNVQYRINGLGVTMDVLNKNQKLIANAFNKALLSIQNGFTATPSALAKIQSVVNANAQALNSLLQQLFNKFGAISSSLQEILSRLDPPEAQVQIDRLINGRLTALNAYVSQQLSDITLIKAGASRAIEKVNECVKSQSPRINFCGNGNHILSLVQNAPYGLLFIHFSYKPTSFKTVLVSPGLCLSGDRGIAPKQGYFIKQNDSWMFTGSSYYYPEPISDKNVVFMNSCSVNFTKAPFIYLNNSIPNLSDFEAELSLWFKNHTSIAPNLTFNSHINATFLDLYYEMNVIQESIKSLNSSFINLKEIGTYEMGSGYIPEAPRDGQAYVRKDGEWVLLSTFL

SEQ ID NO: 16 - protein, 1229 aa, synthetic construct; identical to SEQ ID NO: 15
  REGION 1..1229: note = Synthetic sequence

SEQ ID NO: 17 - protein, 1237 aa, synthetic construct
  REGION 1..1237: note = Synthetic sequence
MFLIIFILPTTLAVIGDFNCTNSFINDYNKTIPRISEDVVDVSLGLGTYYVLNRVYLNTTLLFTGYFPKSGANFRDLALKGSIYLSTLWYKPPFLSDFNNGIFSKVKNTKLYVNNTLYSEFSTIVIGSVFVNTSYTIVVQPHNGILEITACQYTMCEYPHTVCKSKGSIRNESWHIDSSEPLCLFKKNFTYNVSADWLYFHFYQERGVFYAYYADVGMPTTFLFSLYLGTILSHYYVMPLTCNAISSNTDNETLEYWVTPLSRRQYLLNFDEHGVITNAVDCSSSFLSEIQCKTQSFAPNTGVYDLSGFTVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNYDLYGITGQGIFKEVSAAYYNNWQNLLYDSNGNIIGFKDFLTNKTYTILPCYSGRVSAAFYQNSSSPALLYRNLKCSYVLNNISFISQPFYFDSYLGCVLNAVNLTSYSVSSCDLRMGSGFCIDYALPSSRASVGGISSPYRFVTFEPFNVSFVNDSVETVGGLFEIQIPTNFTIAGHEEFIQTSSPKVTIDCSAFVCSNYAACHDLLSEYGTFCDNINSILNEVNDLLDITQLQVANALMQGVTLSSNLNTNLHSDVDNIDFKSLLGCLGSQCGSSSRSPLEDLLFNKVKLSDVGFVEAYNNCTGGSEIRDLLCVQSFNGIKVLPPILSETQISGYTTAATVAAMFPPWSAAAGVPFPLNVQYRINGLGVTMDVLNKNQKLIANAFNKALLSIQNGFTATPSALAKIQSVVNANAQALNSLLQQLFNKFGAISSSLQEILSRLDPPEAQVQIDRLINGRLTALNAYVSQQLSDITLIKAGASRAIEKVNECVKSQSPRINFCGNGNHILSLVQNAPYGLLFIHFSYKPTSFKTVLVSPGLCLSGDRGIAPKQGYFIKQNDSWMFTGSSYYYPEPISDKNVVFMNSCSVNFTKAPFIYLNNSIPNLSDFEAELSLWFKNHTSIAPNLTFNSHINATFLDLYYEMNVIQESIKSLNSSFINLKEIGTYEMGSGYIPEAPRDGQAYVRKDGEWVLLSTFLGSHHHHHH

SEQ ID NO: 18 - protein, 313 aa, synthetic construct
  REGION 1..313: note = Synthetic sequence
MLFVFILFLPSCLGYIGDFRCIQLVNSNGANVSAPSISTETVEVSQGLGTYYVLDRVYLNATLLLTGYYPVDGSKFRNLALTGTNSVSLSWFQPPYLSQFNDGIFAKVQNLKTSTPSGATAYFPTIVIGSLFGYTSYTVVIEPYNGVIMASVCQYTICQLPYTDCKPNTNGNKLIGFWHTDVKPPICVLKRNFTLNVNADAFYFHFYQHGGTFYAYYADKPSATTFLFSVYIGDILTQYYVLPFICNPTAGSTFAPRYWVTPLVKRQYLFNFNQKGVITSAVDCASSYTSEIKCKTQSMLPSTGVYELSGYTV

SEQ ID NO: 19 - protein, 682 aa, synthetic construct
  REGION 1..682: note = Synthetic sequence
CVKYDLYGITGQGVFKEVKADYYNSWQTLLYDVNGNLNGFRDLTTNKTYTIRSCYSGRVSAAFHKDAPEPALLYRNINCSYVFSNNISREENPLNYFDSYLGCVVNADNRTDEALPNCDLRMGAGLCVDYSKSSRASGSVSTGYRLTTFEPYTPMLVNDSVQSVDGLYEMQIPTNFTIGHHEEFIQTRSPKVTIDCAAFVCGDNTACRQQLVEYGSFCVNVNAILNEVNNLLDNMQLQVASALMQGVTISSRLPDGISGPIDDINFSPLLGCIGSTCAEDGNGPSAIRGRSPIEDLLFDKVKLSDVGFVEAYNNCTGGQEVRDLLCVQSFNGIKVLPPVLSESQISGYTTGATAAAMFPPWSAAAGVPFPLSVQYRINGLGVTMNVLSENQKMIASAFNNALGAIQDGFDATPSALGKIQSVVNANAEALNNLLNQLSNRFGAISASLQEILTRLEPPEAKAQIDRLINGRLTALNAYISKQLSDSTLIKVSAAQAIEKVNECVKSQTTRINFCGNGNHILSLVQNAPYGLYFIHFSYVPISFTTANVSPGLCISGDRGLAPKAGYFVQDDGEWKFTGSSYYYPEPITDKNSVIMSSCAVNYTKAPEVFLNTSIPNPPDFKEELDKWFKNQTSIAPDLSLDFEKLNVTLLDLTYEMNRIQDAIKKLNESYINLKEVGTYEMY

SEQ ID NO: 20 - protein, 315 aa, synthetic construct
  REGION 1..315: note = Synthetic sequence
MFLILLISLPTAFAVIGDLKCTTVSINDVDTGVPSISTDTVDVTNGLGTYYVLDRVYLNTTLLLNGYYPTSGSTYRNMALKGTLLLSTLWFKPPFLSDFTNGIFAKVKNTKVIKDGVMYSEFPAITIGSTFVNTSYSVVVQPHTTILGNKLQGFLEISVCQYTMCEYPNTICNPNLGNQRVELWHWDTGVVSCLYKRNFTYDVNADYLYFHFYQEGGTFYAYFTDTGVVTKFLFNVYLGTVLSHYYVMPLTCNSALTLEYWVTPLTSKQYLLAFNQDGVIFNAVDCKSDFMSEIKCKTLSIAPSTGVYELNGYTV

SEQ ID NO: 21 - protein, 673 aa, synthetic construct
  REGION 1..673: note = Synthetic sequence
CVNYDLYGITGQGIFVEVNATYYNSWQNLLYDSNGNLYGFRDYLTNRTFMIRSCYSGRVSAAFHANSSEPALLFRNIKCNYVFNNTLSRQLQPINYFDSYLGCVVNADNSTSSVVQTCDLTVGSGYCVDYSTKSRASGSITTGYRFTNFEPFTVNSVNDSLEPVGGLYEIQIPSEFTIGNMEEFIQTSSPKVTIDCSAFVCGDYAACKSQLVEYGSFCDNINAILTEVNELLDTTQLQVANSLMNGVTLSTKLKDGVNFNVDDINFSPVLGCLGSDCNKVSSRSPIEDLLFSKVKLSDVGFVEAYNNCTGGAEIRDLICVQSYNGIKVLPPLLSENQISGYTLAATSASLFPPWSAAAGVPFPLNVQYRINGIGVTMDVLSQNQKLIANAFNNALGAIQEGFDATPSALVKIQAVVNANAEALNNLLQQLSNRFGAISSSLQEILSRLDPPEAQAQIDRLINGRLTALNAYVSQQLSDSTLVKFSAAQAMEKVNECVKSQSSRINFCGNGNHIISLVQNAPYGLYFIHFSYVPTKYVTAKVSPGLCIAGDRGIAPKSGYFVNVNNTWMFTGSGYYYPEPITGNNVVVMSTCAVNYTKAPDVMLNISTPNLPYFKEELDQWFKNQTSVAPDLSLDYINVTFLDLQDEMNRLQEAIKVLNQSYINLKDIGTYEYY

SEQ ID NO: 22 - protein, 217 aa, synthetic construct
  REGION 1..217: note = Synthetic sequence
QPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNK

SEQ ID NO: 23 - protein, 217 aa, synthetic construct
  REGION 1..217: note = Synthetic sequence
QPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNK

SEQ ID NO: 24 - protein, 1229 aa, synthetic construct
  REGION 1..1229: note = Synthetic sequence
MFLIIFILPTTLAVIGDFNCTNSFINDYNKTIPRISEDVVDVSLGLGTYYVLNRVYLNTTLLFTGYFPKSGANFRDLALKGSIYLSTLWYKPPFLSDFNNGIFSKVKNTKLYVNNTLYSEFSTIVIGSVFVNTSYTIVVQPHNGILEITACQYTMCEYPHTVCKSKGSIRNESWHIDSSEPLCLFKKNFTYNVSADWLYFHFYQERGVFYAYYADVGMPTTFLFSLYLGTILSHYYVMPLTCNAISSNTDNETLEYWVTPLSRRQYLLNFDEHGVITNAVDCSSSFLSEIQCKTQSFAPNTGVYDLSGFTVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNYDLYGITGQGIFKEVSAAYYNNWQNLLYDSNGNIIGFKDFLTNKTYTILPCYSGRVSAAFYQNSSSPALLYRNLKCSYVLNNISFISQPFYFDSYLGCVLNAVNLTSYSVSSCDLRMGSGFCIDYALPSSRASVGGISSPYRFVTFEPFNVSFVNDSVETVGGLFEIQIPTNFTIAGHEEFIQTSSPKVTIDCSAFVCSNYAACHDLLSEYGTFCDNINSILNEVNDLLDITQLQVANALMQGVTLSSNLNTNLHSDVDNIDFKSLLGCLGSQCGSSSRSPLEDLLFNKVKLSDVGFVEAYNNCTGGSEIRDLLCVQSFNGIKVLPPILSETQISGYTTAATVAAMFPPWSAAAGVPFPLNVQYRINGLGVTMDVLNKNQKLIANAFNKALLSIQNGFTATPSALAKIQSVVNANAQALNSLLQQLFNKFGAISSSLQEILSRLDPPEAQVQIDRLINGRLTALNAYVSQQLSDITLIKAGASRAIEKVNECVKSQSPRINFCGNGNHILSLVQNAPYGLLFIHFSYKPTSFKTVLVSPGLCLSGDRGIAPKQGYFIKQNDSWMFTGSSYYYPEPISDKNVVFMNSCSVNFTKAPFIYLNNSIPNLSDFEAELSLWFKNHTSIAPNLTFNSHINATFLDLYYEMNVIQESIKSLNSSFINLKEIGTYEMGSGYIPEAPRDGQAYVRKDGEWVLLSTFL

SEQ ID NO: 25 - protein, 1253 aa, synthetic construct
  REGION 1..1253: note = Synthetic sequence
MFLIIFILPTTLAVIGDFNCTNSFINDYNKTIPRISEDVVDVSLGLGTYYVLNRVYLNTTLLFTGYFPKSGANFRDLALKGSIYLSTLWYKPPFLSDFNNGIFSKVKNTKLYVNNTLYSEFSTIVIGSVFVNTSYTIVVQPHNGILEITACQYTMCEYPHTVCKSKGSIRNESWHIDSSEPLCLFKKNFTYNVSADWLYFHFYQERGVFYAYYADVGMPTTFLFSLYLGTILSHYYVMPLTCNAISSNTDNETLEYWVTPLSRRQYLLNFDEHGVITNAVDCSSSFLSEIQCKTQSFAPNTGVYDLSGFTVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNYDLYGITGQGIFKEVSAAYYNNWQNLLYDSNGNIIGFKDFLTNKTYTILPCYSGRVSAAFYQNSSSPALLYRNLKCSYVLNNISFISQPFYFDSYLGCVLNAVNLTSYSVSSCDLRMGSGFCIDYALPSSRASVGGISSPYRFVTFEPFNVSFVNDSVETVGGLFEIQIPTNFTIAGHEEFIQTSSPKVTIDCSAFVCSNYAACHDLLSEYGTFCDNINSILNEVNDLLDITQLQVANALMQGVTLSSNLNTNLHSDVDNIDFKSLLGCLGSQCGSSSRSPLEDLLFNKVKLSDVGFVEAYNNCTGGSEIRDLLCVQSFNGIKVLPPILSETQISGYTTAATVAAMFPPWSAAAGVPFPLNVQYRINGLGVTMDVLNKNQKLIANAFNKALLSIQNGFTATPSALAKIQSVVNANAQALNSLLQQLFNKFGAISSSLQEILSRLDPPEAQVQIDRLINGRLTALNAYVSQQLSDITLIKAGASRAIEKVNECVKSQSPRINFCGNGNHILSLVQNAPYGLLFIHFSYKPTSFKTVLVSPGLCLSGDRGIAPKQGYFIKQNDSWMFTGSSYYYPEPISDKNVVFMNSCSVNFTKAPFIYLNNSIPNLSDFEAELSLWFKNHTSIAPNLTFNSHINATFLDLYYEMNVIQESIKSLNSSFINLKEIGTYEMGSGYIPEAPRDGQAYVRKDGEWVLLSTFLGSGLNDIFEAQKIEWHEGHHHHHH

SEQ ID NO: 26 - protein, 1279 aa, synthetic construct
  REGION 1..1279: note = Synthetic sequence
MFLIIFILPTTLAVIGDFNCTNSFINDYNKTIPRISEDVVDVSLGLGTYYVLNRVYLNTTLLFTGYFPKSGANFRDLALKGSIYLSTLWYKPPFLSDFNNGIFSKVKNTKLYVNNTLYSEFSTIVIGSVFVNTSYTIVVQPHNGILEITACQYTMCEYPHTVCKSKGSIRNESWHIDSSEPLCLFKKNFTYNVSADWLYFHFYQERGVFYAYYADVGMPTTFLFSLYLGTILSHYYVMPLTCNAISSNTDNETLEYWVTPLSRRQYLLNFDEHGVITNAVDCSSSFLSEIQCKTQSFAPNTGVYDLSGFTVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNYDLYGITGQGIFKEVSAAYYNNWQNLLYDSNGNIIGFKDFLTNKTYTILPCYSGRVSAAFYQNSSSPALLYRNLKCSYVLNNISFISQPFYFDSYLGCVLNAVNLTSYSVSSCDLRMGSGFCIDYALPSSRASVGGISSPYRFVTFEPFNVSFVNDSVETVGGLFEIQIPTNFTIAGHEEFIQTSSPKVTIDCSAFVCSNYAACHDLLSEYGTFCDNINSILNEVNDLLDITQLQVANALMQGVTLSSNLNTNLHSDVDNIDFKSLLGCLGSQCGSSSRSPLEDLLFNKVKLSDVGFVEAYNNCTGGSEIRDLLCVQSFNGIKVLPPILSETQISGYTTAATVAAMFPPWSAAAGVPFPLNVQYRINGLGVTMDVLNKNQKLIANAFNKALLSIQNGFTATPSALAKIQSVVNANAQALNSLLQQLFNKFGAISSSLQEILSRLDPPEAQVQIDRLINGRLTALNAYVSQQLSDITLIKAGASRAIEKVNECVKSQSPRINFCGNGNHILSLVQNAPYGLLFIHFSYKPTSFKTVLVSPGLCLSGDRGIAPKQGYFIKQNDSWMFTGSSYYYPEPISDKNVVFMNSCSVNFTKAPFIYLNNSIPNLSDFEAELSLWFKNHTSIAPNLTFNSHINATFLDLYYEMNVIQESIKSLNSSFINLKEIGTYEMGSGYIPEAPRDGQAYVRKDGEWVLLSTFLGSGYIPEAPRDGQAYVRKDGEWVLLSTFLGGGGSGGGGSGGGGSHHHHHH

SEQ ID NO: 27 - protein, 1229 aa, synthetic construct
  REGION 1..1229: note = Synthetic sequence
MFLIIFILPTTLAVIGDFNCTNSFINDYNKTIPRISEDVVDVSLGLGTYYVLNRVYLNTTLLFTGYFPKSGANFRDLALKGSIYLSTLWYKPPFLSDFNNGIFSKVKNTKLYVNNTLYSEFSTIVIGSVFVNTSYTIVVQPHNGILEITACQYTMCEYPHTVCKSKGSIRNESWHIDSSEPLCLFKKNFTYNVSADWLYFHFYQERGVFYAYYADVGMPTTFLFSLYLGTILSHYYVMPLTCNAISSNTDNETLEYWVTPLSRRQYLLNFDEHGVITNAVDCSSSFLSEIQCKTQSFAPNTGVYDLSGFTVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNYDLYGITGQGIFKEVSAAYYNNWQNLLYDSNGNIIGFKDFLTNKTYTILPCYSGRVSAAFYQNSSSPALLYRNLKCSYVLNNISFISQPFYFDSYLGCVLNAVNLTSYSVSSCDLRMGSGFCIDYALPSSRASVGGISSPYRFVTFEPFNVSFVNDSVETVGGLFEIQIPTNFTIAGHEEFIQTSSPKVTIDCSAFVCSNYAACHDLLSEYGTFCDNINSILNEVNDLLDITQLQVANALMQGVTLSSNLNTNLHSDVDNIDFKSLLGCLGSQCGSSSRSPLEDLLFNKVKLSDVGFVEAYNNCTGGSEIRDLLCVQSFNGIKVLPPILSETQISGYTTAATVAAMFPPWSAAAGVPFPLNVQYRINGLGVTMDVLNKNQKLIANAFNKALLSIQNGFTATPSALAKIQSVVNANAQALNSLLQQLFNKFGAISSSLQEILSRLDPPEAQVQIDRLINGRLTALNAYVSQQLSDITLIKAGASRAIEKVNECVKSQSPRINFCGNGNHILSLVQNAPYGLLFIHFSYKPTSFKTVLVSPGLCLSGDRGIAPKQGYFIKQNDSWMFTGSSYYYPEPISDKNVVFMNSCSVNFTKAPFIYLNNSIPNLSDFEAELSLWFKNHTSIAPNLTFNSHINATFLDLYYEMNVIQESIKSLNSSFINLKEIGTYEMGSGYIPEAPRDGQAYVRKDGEWVLLSTFL

SEQ ID NO: 28 - protein, 1253 aa, synthetic construct
  REGION 1..1253: note = Synthetic sequence
MFLIIFILPTTLAVIGDFNCTNSFINDYNKTIPRISEDVVDVSLGLGTYYVLNRVYLNTTLLFTGYFPKSGANFRDLALKGSIYLSTLWYKPPFLSDFNNGIFSKVKNTKLYVNNTLYSEFSTIVIGSVFVNTSYTIVVQPHNGILEITACQYTMCEYPHTVCKSKGSIRNESWHIDSSEPLCLFKKNFTYNVSADWLYFHFYQERGVFYAYYADVGMPTTFLFSLYLGTILSHYYVMPLTCNAISSNTDNETLEYWVTPLSRRQYLLNFDEHGVITNAVDCSSSFLSEIQCKTQSFAPNTGVYDLSGFTVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNYDLYGITGQGIFKEVSAAYYNNWQNLLYDSNGNIIGFKDFLTNKTYTILPCYSGRVSAAFYQNSSSPALLYRNLKCSYVLNNISFISQPFYFDSYLGCVLNAVNLTSYSVSSCDLRMGSGFCIDYALPSSRASVGGISSPYRFVTFEPFNVSFVNDSVETVGGLFEIQIPTNFTIAGHEEFIQTSSPKVTIDCSAFVCSNYAACHDLLSEYGTFCDNINSILNEVNDLLDITQLQVANALMQGVTLSSNLNTNLHSDVDNIDFKSLLGCLGSQCGSSSRSPLEDLLFNKVKLSDVGFVEAYNNCTGGSEIRDLLCVQSFNGIKVLPPILSETQISGYTTAATVAAMFPPWSAAAGVPFPLNVQYRINGLGVTMDVLNKNQKLIANAFNKALLSIQNGFTATPSALAKIQSVVNANAQALNSLLQQLFNKFGAISSSLQEILSRLDPPEAQVQIDRLINGRLTALNAYVSQQLSDITLIKAGASRAIEKVNECVKSQSPRINFCGNGNHILSLVQNAPYGLLFIHFSYKPTSFKTVLVSPGLCLSGDRGIAPKQGYFIKQNDSWMFTGSSYYYPEPISDKNVVFMNSCSVNFTKAPFIYLNNSIPNLSDFEAELSLWFKNHTSIAPNLTFNSHINATFLDLYYEMNVIQESIKSLNSSFINLKEIGTYEMGSGYIPEAPRDGQAYVRKDGEWVLLSTFLGSGLNDIFEAQKIEWHEGHHHHHH

SEQ ID NO: 29 - protein, 1279 aa, synthetic construct
  REGION 1..1279: note = Artificial sequence
MFLIIFILPTTLAVIGDFNCTNSFINDYNKTIPRISEDVVDVSLGLGTYYVLNRVYLNTTLLFTGYFPKSGANFRDLALKGSIYLSTLWYKPPFLSDFNNGIFSKVKNTKLYVNNTLYSEFSTIVIGSVFVNTSYTIVVQPHNGILEITACQYTMCEYPHTVCKSKGSIRNESWHIDSSEPLCLFKKNFTYNVSADWLYFHFYQERGVFYAYYADVGMPTTFLFSLYLGTILSHYYVMPLTCNAISSNTDNETLEYWVTPLSRRQYLLNFDEHGVITNAVDCSSSFLSEIQCKTQSFAPNTGVYDLSGFTVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNYDLYGITGQGIFKEVSAAYYNNWQNLLYDSNGNIIGFKDFLTNKTYTILPCYSGRVSAAFYQNSSSPALLYRNLKCSYVLNNISFISQPFYFDSYLGCVLNAVNLTSYSVSSCDLRMGSGFCIDYALPSSRASVGGISSPYRFVTFEPFNVSFVNDSVETVGGLFEIQIPTNFTIAGHEEFIQTSSPKVTIDCSAFVCSNYAACHDLLSEYGTFCDNINSILNEVNDLLDITQLQVANALMQGVTLSSNLNTNLHSDVDNIDFKSLLGCLGSQCGSSSRSPLEDLLFNKVKLSDVGFVEAYNNCTGGSEIRDLLCVQSFNGIKVLPPILSETQISGYTTAATVAAMFPPWSAAAGVPFPLNVQYRINGLGVTMDVLNKNQKLIANAFNKALLSIQNGFTATPSALAKIQSVVNANAQALNSLLQQLFNKFGAISSSLQEILSRLDPPEAQVQIDRLINGRLTALNAYVSQQLSDITLIKAGASRAIEKVNECVKSQSPRINFCGNGNHILSLVQNAPYGLLFIHFSYKPTSFKTVLVSPGLCLSGDRGIAPKQGYFIKQNDSWMFTGSSYYYPEPISDKNVVFMNSCSVNFTKAPFIYLNNSIPNLSDFEAELSLWFKNHTSIAPNLTFNSHINATFLDLYYEMNVIQESIKSLNSSFINLKEIGTYEMGSGYIPEAPRDGQAYVRKDGEWVLLSTFLGSGYIPEAPRDGQAYVRKDGEWVLLSTFLGGGGSGGGGSGGGGSHHHHHH

(19) United States
(12) Patent Application Publication
(10) Pub. No.: US 2025/0026793 A1
(43) Pub. Date: Jan. 23, 2025

(54) CHIMERIC BETACORONAVIRUS SPIKE POLYPEPTIDES

(71) Applicant: THE UNIVERSITY OF MELBOURNE, Melbourne, Victoria (AU)

(72) Inventors: Adam Kenneth WHEATLEY, Melbourne, Victoria (AU); Hyon Xhi TAN, Melbourne, Victoria (AU); Jennifer Ann JUNO, Melbourne, Victoria (AU); Stephen John KENT, Melbourne, Victoria (AU)

(21) Appl. No.: 18/703,126

(22) PCT Filed: Oct. 21, 2022
(86) PCT No.: PCT/AU2022/051266

§ 371 Date: Apr. 19, 2024

(30) Foreign Application Priority Data
Oct. 21, 2021 (AU) 2021903377

(51) Int. Cl. C07K 14/005; A61K 39/215; A61P 31/14

(52) U.S. Cl. CPC C07K 14/005; A61K 39/215; A61P 31/14; C12N 2770/20022; C12N 2770/20034; C12N 2770/20071

(57) ABSTRACT

The present invention relates to chimeric coronavirus spike polypeptides and uses thereof, including in vaccines.

### TECHNICAL FIELD

The present invention relates to chimeric coronavirus spike polypeptides and uses thereof, including in vaccines.

### BACKGROUND OF INVENTION

The global spread of SARS-COV-2 has sparked intense global research efforts to combat the significant health and economic impacts of the pandemic. Monoclonal antibodies (mAbs) have demonstrated promise as treatments or prophylactic agents against other viral diseases such as RSV and Ebola.

The clinical development of SARS-COV-2 antibody-based therapeutics has seen rapid progression of candidate mAbs through clinical trials, with two human mAb cocktails (casirivimab/imdevimab and bamlanivimab/etesevimab) and one monotherapy (bamlanivimab) conditionally approved for treatment of high-risk ambulatory patients. However, some of these first-generation treatments suffer significant losses of neutralisation potency in the face of ongoing viral evolution, with near complete loss of activity against B.1.351 and P.1 SARS-COV-2 variants of concern (VOC) reported for many neutralising human mAbs. The loss of potency of antibodies against VOC suggests vaccine strategies using the original (wild-type or ancestral) strain will have reduced efficacy against VOC.

Indeed, vaccine efficacy against the B.1.351 strain, first identified in South Africa and now spreading globally, is significantly reduced in clinical trials compared to WT virus for Novavax (protein; 96.5% reduced to 60%), AstraZeneca (adenovirus; 62% reduced to 10%) and Johnson & Johnson vaccines (adenovirus; 72% reduced to 52%). In vitro studies suggest significant reductions in serum neutralising activity against B.1.351 and the P.1 strain (first identified in Brazil) in subjects receiving first generation vaccines. VOC show reduced binding by a major fraction of RBD-specific monoclonal antibodies and near total loss of NTD-specific antibodies, explaining the significant loss of protective vaccine efficacy. Loss of protection is likely to be exacerbated as antibody titres wane.

There is a need for methods to elicit improved immune responses against the SARS-COV-2 spike, including to the spike of SARS-COV-2 VOC.

### SUMMARY OF INVENTION

In one aspect, the present invention provides a chimeric polypeptide comprising an amino acid sequence encoding a N terminal domain of a first betacoronavirus spike protein, an amino acid sequence encoding one or more immunogenic epitope of a receptor binding domain of a second betacoronavirus spike protein, and an amino acid sequence encoding a C terminal region of the first coronavirus spike protein.

In a second aspect, the present invention provides chimeric polypeptide comprising an amino acid sequence encoding a N terminal domain of a first betacoronavirus spike protein, an amino acid sequence encoding a receptor binding domain of a second betacoronavirus spike protein, and an amino acid sequence encoding a C terminal region of the first coronavirus spike protein.

In one embodiment, the present invention provides a chimeric polypeptide as described herein, wherein the amino acid sequence encoding the C terminal region of the first coronavirus spike protein encodes the S2′ protease cleavage site, fusion peptide, heptad repeat 1, central helix, connector domain and heptad repeat 2 regions of the first betacoronavirus spike protein.

In another embodiment, the present invention provides a chimeric polypeptide as described herein, wherein the second betacoronavirus spike protein is a SARS-COV, MERS-COV, or SARS-COV-2 betacoronavirus spike protein.

In a further embodiment, the present invention provides a chimeric polypeptide as described herein, wherein the first betacoronavirus spike protein is selected from the group consisting of a HCoVHKU1 betacoronavirus spike protein, an ECoV betacoronavirus spike protein, and a MHV spike protein.

In a further embodiment, the present invention provides a chimeric polypeptide as described herein, wherein the second betacoronavirus spike protein is selected from the group consisting of a B.1.1.7 and Q (Alpha), B.1.351 (Beta), B.1.617.2 and AY (Delta), P.1 (Gamma), B.1.427 (Epsilon), B.1.429 (Epsilon), B.1.525 (Eta), B.1.526 (Iota) B.1.617.1 (Kappa), B.1.617.3, C.37 (Lambda), B.1.621 (Mu), B.1.1.529 (Omicron), and P.2 (Zeta) spike protein.

In a further embodiment, the present invention provides a chimeric polypeptide as described herein, wherein the second betacoronavirus spike protein is selected from the group consisting of a B.1.1.529, BA.1, BA.1.1, BA.2, BA.3, BA.4 and BA.5 spike protein.

In a further embodiment, the present invention provides a chimeric polypeptide as described herein, wherein the chimeric polypeptide further comprises an amino acid sequence encoding a trimerization domain.

In a further embodiment, the present invention provides a chimeric polypeptide as described herein, wherein the trimerization domain is the C-terminal domain of T4 fibritin.

In a further embodiment, the present invention provides a chimeric polypeptide as described herein, wherein the amino acid sequence encoding the N terminal domain domain is selected from the group consisting of:

(i)
MFLIIFILPTTLAVIGDFNCTNSFINDYNKTIPRISEDVVDVSLGLGTYY
VLNRVYLNTTLLFTGYFPKSGANFRDLALKGSIYLSTLWYKPPFLSDFNN
GIFSKVKNTKLYVNNTLYSEFSTIVIGSVFVNTSYTIVVQPHNGILEITA
CQYTMCEYPHTVCKSKGSIRNESWHIDSSEPLCLFKKNFTYNVSADWLYF
HFYQERGVFYAYYADVGMPTTFLFSLYLGTILSHYYVMPLTCNAISSNTD
NETLEYWVTPLSRRQYLLNFDEHGVITNAVDCSSSFLSEIQCKTQSFAPN
TGVYDLSGFTV or a variant thereof;
(ii)
MFLILLISLPTAFAVIGDLKCTTVSINDVDTGVPSISTDTVDVTNGLGTY
YVLDRVYLNTTLLLNGYYPTSGSTYRNMALKGTLLLSTLWFKPPFLSDFT
NGIFAKVKNTKVIKDGVMYSEFPAITIGSTFVNTSYSVVVQPHTTILGNK
LQGFLEISVCQYTMCEYPNTICNPNLGNQRVELWHWDTGVVSCLYKRNFT
YDVNADYLYFHFYQEGGTFYAYFTDTGVVTKFLFNVYLGTVLSHYYVMPL
TCNSALTLEYWVTPLTSKQYLLAFNQDGVIFNAVDCKSDFMSEIKCKTLS
IAPSTGVYELNGYTV or a variant thereof;
and
(iii)
MLFVFILFLPSCLGYIGDFRCIQLVNSNGANVSAPSISTETVEVSQGLGT
YYVLDRVYLNATLLLTGYYPVDGSKFRNLALTGTNSVSLSWFQPPYLSQF
NDGIFAKVQNLKTSTPSGATAYFPTIVIGSLFGYTSYTVVIEPYNGVIMA
SVCQYTICQLPYTDCKPNTNGNKLIGFWHTDVKPPICVLKRNFTLNVNAD
AFYFHFYQHGGTFYAYYADKPSATTFLFSVYIGDILTQYYVLPFICNPTA
GSTFAPRYWVTPLVKRQYLFNFNQKGVITSAVDCASSYTSEIKCKTQSML
PSTGVYELSGYTV.

In a further embodiment, the present invention provides a chimeric polypeptide as described herein, wherein the amino acid sequence encoding the C terminal region is selected from the group consisting of:

(i)
CVNYDLYGITGQGIFKEVSAAYYNNWQNLLYDSNGNIIGFKDFLINKTYTILPCYSGR
VSAAFYQNSSSPALLYRNLKCSYVLNNISFISQPFYFDSYLGCVLNAVNLTSYSVSSC
DLRMGSGFCIDYALPSSRASVGGISSPYRFVTFEPFNVSFVNDSVETVGGLFEIQIPT
NFTIAGHEEFIQTSSPKVTIDCSAFVCSNYAACHDLLSEYGTFCDNINSILNEVNDLLDI
TQLQVANALMQGVTLSSNLNTNLHSDVDNIDFKSLLGCLGSQCGSSSRSPLEDLLFN
KVKLSDVGFVEAYNNCTGGSEIRDLLCVQSFNGIKVLPPILSETQISGYTTAATVAAM
FPPWSAAAGVPFPLNVQYRINGLGVTMDVLNKNQKLIANAFNKALLSIQNGFTATPS
ALAKIQSVVNANAQALNSLLQQLFNKFGAISSSLQEILSRLDPPEAQVQIDRLINGRLT
ALNAYVSQQLSDITLIKAGASRAIEKVNECVKSQSPRINFCGNGNHILSLVQNAPYGL
LFIHFSYKPTSFKTVLVSPGLCLSGDRGIAPKQGYFIKQNDSWMFTGSSYYYPEPISD
KNVVFMNSCSVNFTKAPFIYLNNSIPNLSDFEAELSLWFKNHTSIAPNLTENSHINATF
LDLYYEMNVIQESIKSLNSSFINLKEIGTYEM
or
a variant thereof;
(ii)
CVNYDLYGITGQGIFVEVNATYYNSWQNLLYDSNGNLYGFRDYLTNRTFMIRSCYS
GRVSAAFHANSSEPALLFRNIKCNYVFNNTLSRQLQPINYFDSYLGCVVNADNSTSS
VVQTCDLTVGSGYCVDYSTKSRASGSITTGYRFTNFEPFTVNSVNDSLEPVGGLYEI
QIPSEFTIGNMEEFIQTSSPKVTIDCSAFVCGDYAACKSQLVEYGSFCDNINAILTEVN
ELLDTTQLQVANSLMNGVTLSTKLKDGVNFNVDDINFSPVLGCLGSDCNKVSSRSPI
EDLLFSKVKLSDVGFVEAYNNCTGGAEIRDLICVQSYNGIKVLPPLLSENQISGYTLAA
TSASLFPPWSAAAGVPFPLNVQYRINGIGVTMDVLSQNQKLIANAFNNALGAIQEGF
DATPSALVKIQAVVNANAEALNNLLQQLSNRFGAISSSLQEILSRLDPPEAQAQIDRLI
NGRLTALNAYVSQQLSDSTLVKFSAAQAMEKVNECVKSQSSRINFCGNGNHIISLVQ
NAPYGLYFIHFSYVPTKYVTAKVSPGLCIAGDRGIAPKSGYFVNVNNTWMFTGSGYY
YPEPITGNNVVVMSTCAVNYTKAPDVMLNISTPNLPYFKEELDQWFKNQTSVAPDLS
LDYINVTFLDLQDEMNRLQEAIKVLNQSYINLKDIGTYEYY
or
a variant thereof;
and
(iii)
CVKYDLYGITGQGVFKEVKADYYNSWQTLLYDVNGNLNGFRDLTTNKTYTIRSCYS
GRVSAAFHKDAPEPALLYRNINCSYVFSNNISREENPLNYFDSYLGCVVNADNRTDE
ALPNCDLRMGAGLCVDYSKSSRASGSVSTGYRLTTFEPYTPMLVNDSVQSVDGLYE
MQIPTNFTIGHHEEFIQTRSPKVTIDCAAFVCGDNTACRQQLVEYGSFCVNVNAILNE
VNNLLDNMQLQVASALMQGVTISSRLPDGISGPIDDINFSPLLGCIGSTCAEDGNGPS
AIRGRSPIEDLLFDKVKLSDVGFVEAYNNCTGGQEVRDLLCVQSFNGIKVLPPVLSES
QISGYTTGATAAAMFPPWSAAAGVPFPLSVQYRINGLGVTMNVLSENQKMIASAFN
NALGAIQDGFDATPSALGKIQSVVNANAEALNNLLNQLSNRFGAISASLQEILTRLEP
PEAKAQIDRLINGRLTALNAYISKQLSDSTLIKVSAAQAIEKVNECVKSQTTRINFCGN
GNHILSLVQNAPYGLYFIHFSYVPISFTTANVSPGLCISGDRGLAPKAGYFVQDDGE
WKFTGSSYYYPEPITDKNSVIMSSCAVNYTKAPEVFLNTSIPNPPDFKEELDKWFKN
QTSIAPDLSLDFEKLNVTLLDLTYEMNRIQDAIKKLNESYINLKEVGTYEMY

In a further embodiment, the present invention provides a chimeric polypeptide as described herein, wherein the amino acid sequence encoding the RBD domain is selected from the group consisting of:

QPTESIVRFPNITNLCPFGEVFNATRFASVYAWNRKRISNCVADYSVLYNSASFSTFK
CYGVSPTKLNDLCFTNVYADSFVIRGDEVRQIAPGQTGKIADYNYKLPDDFTGCVIA
WNSNNLDSKVGGNYNYLYRLFRKSNLKPFERDISTEIYQAGSTPCNGVEGFNCYFP
LQSYGFQPTNGVGYQPYRVVVLSFELLHAPATVCGPKKSTNLVKNK
or
a variant thereof;
QPTESIVRFPNITNLCPFGEVFNATRFASVYAWNRKRISNCVADYSVLYNSASFSTFK
CYGVSPTKLNDLCFTNVYADSFVIRGDEVRQIAPGQTGNIADYNYKLPDDFTGCVIA
WNSNNLDSKVGGNYNYLYRLFRKSNLKPFERDISTEIYQAGSTPCNGVEGENCYFP
LQSYGFQPTYGVGYQPYRVVVLSFELLHAPATVCGPKKSTNLVKNK
or
a variant thereof;
QPTESIVRFPNITNLCPFGEVFNATRFASVYAWNRKRISNCVADYSVLYNSASFSTFK
CYGVSPTKLNDLCFTNVYADSFVIRGDEVRQIAPGQTGNIADYNYKLPDDFTGCVIA
WNSNNLDSKVGGNYNYLYRLFRKSNLKPFERDISTEIYQAGSTPCNGVKGFNCYFP
LQSYGFQPTYGVGYQPYRVVVLSFELLHAPATVCGPKKSTNLVKNK
or
a variant thereof;
QPTESIVRFPNITNLCPFGEVFNATRFASVYAWNRKRISNCVADYSVLYNSASFSTFK
CYGVSPTKLNDLCFTNVYADSFVIRGDEVRQIAPGQTGTIADYNYKLPDDFTGCVIA
WNSNNLDSKVGGNYNYLYRLFRKSNLKPFERDISTEIYQAGSTPCNGVKGFNCYFP
LQSYGFQPTYGVGYQPYRVVVLSFELLHAPATVCGPKKSTNLVKNK
or
a variant thereof;
QPTESIVRFPNITNLCPFGEVFNATRFASVYAWNRKRISNCVADYSVLYNSASFSTFK
CYGVSPTKLNDLCFTNVYADSFVIRGDEVRQIAPGQTGKIADYNYKLPDDFTGCVIA
WNSNNLDSKVGGNYNYRYRLFRKSNLKPFERDISTEIYQAGSKPCNGVEGENCYFP
LQSYGFQPTNGVGYQPYRVVVLSFELLHAPATVCGPKKSTNLVKNK;
or
a variant thereof;
QPTESIVRFPNITNLCPFGEVFNATRFASVYAWNRKRISNCVADYSVLYNSASFSTFK
CYGVSPTKLNDLCFTNVYADSFVIRGDEVRQIAPGQTGKIADYNYKLPDDFTGCVIA
WNSNNLDSKVGGNYNYLYRLFRKSNLKPFERDISTEIYQAGSTPCNGVKGFNCYFP
LQSYGFQPTNGVGYQPYRVVVLSFELLHAPATVCGPKKSTNLVKNK
or
a variant thereof;
QPTESIVRFPNITNLCPFGEVFNATRFASVYAWNRKRISNCVADYSVLYNSASFSTFK
CYGVSPTKLNDLCFTNVYADSFVIRGDEVRQIAPGQTGKIADYNYKLPDDFTGCVIA
WNSNNLDSKVGGNYNYRYRLFRKSNLKPFERDISTEIYQAGSTPCNGVQGFNCYFP
LQSYGFQPTNGVGYQPYRVVVLSFELLHAPATVCGPKKSTNLVKNK;
or
a variant thereof;
QPTESIVRFPNITNLCPFGEVFNATRFASVYAWNRKRISNCVADYSVLYNSASFSTFK
CYGVSPTKLNDLCFTNVYADSFVIRGDEVRQIAPGQTGKIADYNYKLPDDFTGCVIA
WNSNNLDSKVGGNYNYQYRLFRKSNLKPFERDISTEIYQAGSTPCNGVEGENCYSP
LQSYGFQPTNGVGYQPYRVVVLSFELLHAPATVCGPKKSTNLVKNK;
or
a variant thereof;
QPTESIVRFPNITNLCPFGEVFNATRFASVYAWNRKRISNCVADYSVLYNSASFSTFK
CYGVSPTKLNDLCFTNVYADSFVIRGDEVRQIAPGQTGNIADYNYKLPDDFTGCVIA
WNSNNLDSKVGGNYNYRYRLFRKSNLKPFERDISTEIYQAGSKPCNGVKGFNCYFP
LQSYGFQPTYGVGYQPYRVVVLSFELLHAPATVCGPKKSTNLVKNK;
or
a variant thereof;
QPTESIVRFPNITNLCPFGEVFNATRFASVYAWNRKRISNCVADYSVLYNSASFSTFK
CYGVSPTKLNDLCFTNVYADSFVIRGDEVRQIAPGQTGNIADYNYKLPDDFTGCVIA
WNSNNLDSKVGGNYNYRYRLFRKSNLKPFERDISTEIYQAGNTPCNGVKGFNCYFP
LQSYGFQPTYGVGYQPYRVVVLSFELLHAPATVCGPKKSTNLVKNK;
or
a variant thereof;
QPTESIVRFPNITNLCPFDEVFNATRFASVYAWNRKRISNCVADYSVLYNLAPFFTFK
CYGVSPTKLNDLCFTNVYADSFVIRGDEVRQIAPGQTGNIADYNYKLPDDFTGCVIA
WNSNKLDSKVSGNYNYLYRLFRKSNLKPFERDISTEIYQAGNKPCNGVAGENCYFP
LRSYSFRPTYGVGHQPYRVVVLSFELLHAPATVCGPKKSTNLVKNK;
or
a variant thereof;
and
QPTESIVRFPNITNLCPFDEVFNATRFASVYAWNRKRISNCVADYSVLYNFAPFFAFK
CYGVSPTKLNDLCFTNVYADSFVIRGNEVSQIAPGQTGNIADYNYKLPDDFTGCVIA
WNSNKLDSKVGGNYNYLYRLFRKSNLKPFERDISTEIYQAGNKPCNGVAGENCYFP
LRSYGFRPTYGVGHQPYRVVVLSFELLHAPATVCGPKKSTNLVKNK;
or
a variant thereof.

In a further embodiment, the present invention provides a chimeric polypeptide as described herein, wherein the amino acid sequence encoding the one or more immunogenic epitope is a receptor binding domain of the first betacoronavirus spike protein comprising one or more amino acid variant of a second betacoronavirus spike protein.

In a further embodiment, the present invention provides a chimeric polypeptide as described herein, wherein the one or more amino acid variant is selected from an amino acid insertion, deletion or an amino acid substitution.

In a further embodiment, the present invention provides a chimeric polypeptide as described herein, wherein the second betacoronavirus spike protein is selected from the group consisting of a B.1.1.7 and Q (Alpha), B.1.351 (Beta), B.1.617.2 and AY (Delta), P.1 (Gamma), B.1.427 (Epsilon), B.1.429 (Epsilon), B.1.525 (Eta), B.1.526 (Iota) B.1.617.1 (Kappa), B.1.617.3, C.37 (Lambda), B.1.621 (Mu), B.1.1.529 (Omicron), and P.2 (Zeta) spike protein.

In a further embodiment, the present invention provides a chimeric polypeptide as described herein, wherein the second betacoronavirus spike protein is selected from the group consisting of a B.1.1.529, BA.1, BA.1.1, BA.2, BA.3, BA.4 and BA.5 spike protein.

In a further embodiment, the present invention provides a chimeric polypeptide as described herein, wherein the amino acid sequence encoding a trimerization domain is GYIPEAPRDGQAYVRKDGEWVLLSTFL or a variant thereof.

In a further embodiment, the present invention provides a chimeric polypeptide as described herein, wherein the chimeric polypeptide further comprises an amino acid sequence encoding a purification tag.

In another aspect, the present invention provides a nucleic acid encoding a chimeric polypeptide as described herein.

In a further embodiment, the present invention provides a nucleic acid as described herein, wherein the C terminal region of the first coronavirus spike protein further encodes the transmembrane domain and cytoplasmic tail of the first betacoronavirus spike protein.

In a further embodiment, the present invention provides a nucleic acid as described herein, wherein the nucleic acid is mRNA or DNA.

In another aspect, the present invention provides a vector comprising a nucleic acid as described herein.

In another aspect, the present invention provides a host cell comprising a nucleic acid as described herein.

In another aspect, the present invention provides a vaccine composition comprising a chimeric polypeptide described herein or a trimer thereof, or a nucleic acid described herein, and a pharmaceutically acceptable carrier.

In another aspect, the present invention provides a method for prophylaxis or treatment of a betacoronavirus infection in a subject comprising administering an effective amount of a vaccine composition described herein.

In another aspect, the present invention provides a use of a chimeric polypeptide described herein or a trimer thereof, or a nucleic acid described herein in the manufacture of a medicament for prophylaxis or treatment of SARS-COV-2 infection in a subject.

### BRIEF DESCRIPTION OF DRAWINGS

FIG. 1. Chimeric trimeric RBD (CTR) vaccine platform, indicating the SARS-COV-2 RBD (yellow), VOC mutations (red) and HKU1 spike trimer scaffold (blue).

FIG. 2. Structural and antigenic integrity of CTR-WT confirmed using (A) SDS-PAGE, (B) size-exclusion chromatography, and (C) ELISA with a panel of 8 human mAbs binding key neutralising epitopes on the SARS-COV-2 RBD including casirivimab and imdevimab (Regeneron).

FIG. 3. Immunogenicity and protective efficacy of CTR-WT in mice. (A) RBD and (B) neutralising antibody titres in mice immunised with 2 doses of CTR-WT compared to human convalescent plasma (CP). (C) Frequency of CD4 T follicular helper cells recognising the RBD or CTR scaffold among vaccinated mice. (D) Lung viral load after SARS-COV-2 challenge in mice vaccinated with 0, 1 or 2 doses of CTR-WT or SARS-COV-2 spike protein.

FIG. 4. NHP were immunised with two doses of CTR-WT and (A) anti-RBD binding IgG titres and (B) neutralisation activity assessed relative to human convalescent plasma samples (CP).

FIG. 5. A Structural and antigenic integrity of a CTR comprising Omicron BA.2 spike RBD in HKU-1 confirmed using (A) size-exclusion chromatography and (B) SDS-PAGE.

### DETAILED DESCRIPTION

The present invention is based in part on the characterisation of a vaccine platform to focus the immune response onto a pathogenic oronavirus (e.g. SARS-COV-2) spike receptor binding domain (RBD), including the production of potent neutralising antibodies against the spike RBD, using a Chimeric Trimeric RBD (“CTR”) that comprises a highly immunogenic, trimeric scaffold capable of eliciting immunity in either naïve or previously vaccinated/infected populations, and which can be used to incorporate mutations from existing and future VOC.

The present inventors have demonstrated in Example 1 the construction of a Chimeric Trimeric Receptor Binding Domain polypeptide comprising a replacement of the RBD of a first betacoronavirus spike protein with the RBD of a second coronavirus RBD protein, and a trimerization sequence. Example 2 demonstrates that such CTR polypeptides are antigenically intact, and Example 3 demonstrates that CTR polypeptides induce neutralising and anti-RBD antibodies in vivo, and at higher titres than convalescent humans following COVID-19 infection.

Accordingly, in one embodiment, the present invention provides a chimeric polypeptide comprising an amino acid sequence encoding a N terminal domain of a first betacoronavirus spike protein, an amino acid sequence encoding a receptor binding domain of a second betacoronavirus spike protein, and an amino acid sequence encoding a C terminal region of the first coronavirus spike protein.

In another one embodiment, the present invention provides a chimeric polypeptide comprising an amino acid sequence encoding a N terminal domain of a first betacoronavirus spike protein, an amino acid sequence encoding one or more immunogenic epitope of a receptor binding domain of a second betacoronavirus spike protein, and an amino acid sequence encoding a C terminal region of the first coronavirus spike protein.

The spike protein is a transmembrane glycoprotein, which forms homotrimers on the surface of the virion. The SARS-COV-2 spike protein is highly glycosylated, with 66 potential N-glycosylation sites per trimer. The SARS-COV-2 spike protein is post-translationally cleaved by mammalian furin into two subunits: S1 and S2. The S1 subunit largely consists of the amino-terminal domain and the receptor-binding domain (RBD), and is responsible for binding to the host cell-surface receptor, ACE2, whereas the S2 subunit includes the trimeric core of the protein and is responsible for membrane fusion.

Spike proteins contain a number of domains, including a signal sequence (SS), an N-terminal domain (NTD), a receptor binding domain (RBD) which binds to a host cell receptor, subdomains 1 and 2 (SD1 and SD2), a S2′ protease cleavage site (S2′), a fusion peptide (FP) domain, a heptad repeat 1 (HR1) domain, a central helix (CH) domain, a connector domain (CD), a heptad repeat 2 (HR2) domain, a transmembrane (TM) domain; and a cytoplasmic tail (CT).

As will be discussed in more detail below, in one embodiment, the first betacoronavirus spike protein is a spike protein into which a heterologous RBD (e.g. from a second betacoronavirus) can be engineered and be antigenically intact.

Without wishing to be bound by theory, the present inventors propose that the performance of COVID-19 vaccines with whole VOC spike proteins is not guaranteed. Firstly, anti-vector responses will limit the repeated use of viral-vectored vaccines. Secondly, new vaccines will be deployed into a complex landscape of pre-existing immunity from prior infection or previous immunisation. Thus, immune imprinting (original antigenic sin) may confound the ability of booster vaccines to elicit VOC-specific neutralising responses, as evidenced by seasonal influenza vaccines that suffer from suboptimal efficacy with viral changes and repeated annual vaccination [Aydillo, T. et al. (2021) Nature Comms 12, 3781, and Davenport, F. M. and Hennessy, A. V. (1957) J Exp Med 106 (6), 835-50]. In these situations, repeated immunisations with similar but slightly distinct antigens does not drive strong immune responses against mutated epitopes, but instead preferentially recalls responses to epitopes that are shared between the viral strains. As critical VOC mutations lie in the RBD, VOC boosters using whole SAR-COV-2 spike immunogens risk only recalling immunity to conserved epitopes that are not maximally protective. The present inventors have demonstrated herein that a Chimeric Trimeric RBD vaccine comprising a chimeric polypeptide as described herein induces robust CD4 T follicular helper cell responses in vivo, induces near complete suppression of viral replication in the lungs of mice, including durable sterilising protection after a single CTR-WT vaccine injection, and induces neutralising and anti-RBD antibodies in non-human primates, and at higher titres than convalescent humans following COVID-19 infection. The data described in the Examples establishes the principle that the immune response can be focused on the RBD using a whole (trimeric) spike immunogen without the induction of potentially distracting (including non-neutralising) responses to epitopes outside the RBD.

As used herein “immunogenic epitope” refers to an antigenic determinant. An immunogenic epitope includes particular chemical groups or peptide sequences on a molecule (e.g. a SARS-COV-2 spike amino acid sequence) that are antigenic, such that they elicit a specific immune response, for example, an epitope is the region of an antigen to which B and/or T cells respond. Epitopes can be formed both from contiguous amino acids or non-contiguous amino acids juxtaposed by tertiary folding of a protein.

In one embodiment, the first betacoronavirus spike protein is a HCoV-OC43, HCOVHKU1, HCOV-NL63 and HCoV-229, BCOV, RaCoV, MHV, ECOV betacoronavirus spike protein.

Accordingly, a chimeric polypeptide as described herein can comprise an N terminal domain and the C terminal region of the first betacoronavirus spike protein and the RBD region of the second betacoronavirus spike protein.

In a preferred embodiment, the amino acid sequence encoding the N terminal domain is

(SEQ ID NO: 1)
MFLIIFILPTTLAVIGDFNCTNSFINDYNKTIPRISEDVVDVSLGLGTYY
VLNRVYLNTTLLFTGYFPKSGANFRDLALKGSIYLSTLWYKPPFLSDFNN
GIFSKVKNTKLYVNNTLYSEFSTIVIGSVFVNTSYTIVVQPHNGILEITA
CQYTMCEYPHTVCKSKGSIRNESWHIDSSEPLCLFKKNFTYNVSADWLYF
HFYQERGVFYAYYADVGMPTTFLFSLYLGTILSHYYVMPLTCNAISSNTD
NETLEYWVTPLSRRQYLLNFDEHGVITNAVDCSSSFLSEIQCKTQSFAPN
TGVYDLSGFTV or a variant thereof.

In another embodiment, the amino acid sequence encoding the N terminal domain has at least 80% identity to the amino acid sequence of SEQ ID NO: 1.

The term “variant” applies to both amino acid and nucleic acid sequences. With respect to particular nucleic acid sequences, ‘variants’ includes nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For polypeptide sequences, “variants” includes a variant which has conservative amino acid substitutions, amino acid residues replaced with other amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

As used herein, a “chimeric polypeptide” relates to a protein (e.g. a recombinant protein) containing amino acid sequences from at least two unrelated proteins (e.g. a first betacoronavirus spike protein region, and a second betacoronavirus spike protein) that have been joined together, via a peptide bond, to make a single protein. As demonstrated herein, linkers (e.g. GS linkers) can be used to link two unrelated sequences. Accordingly, the unrelated amino acid sequences can be joined directly to each other or they can be joined using a linker sequence.

As used herein, “sequence identity” relates to the similarity of amino acid sequences. The best possible alignment of two sequences is prepared, and the sequence identity is determined by the percentage of identical residues. Standard methods are available for the alignment of sequences, e.g. algorithms of Needleman and Wunsch (J Mol Biol (1970) 48, 443), Smith and Waterman (Adv Appl Math (1981) 2, 482), Pearson and Lipman (Proc Natl Acad Sci USA (1988) 85, 2444), and others. Suitable software is commercially available, e.g. the GCG suite of software (Devereux et al (1984), Nucl Acids Res 12, 387), where alignments can be produced using, for example, GAP or BESTFIT with default parameters, or successors thereof. The Blast algorithm, originally described by Altschul et al (J. Mol. Biol. (1990) 215, 403), but further refined to include gapped alignments (Blast 2), available from various sources such as the EBI, NCBI, will also produce alignments and calculate the % identity between two sequences.

In another embodiment, the amino acid sequence encoding the N terminal domain has at least 90% identity to the amino acid sequence of SEQ ID NO: 1.

In one embodiment of the present invention, the amino acid sequence encoding the C terminal region of the first coronavirus spike protein encodes the S2′ protease cleavage site, fusion peptide, heptad repeat 1, central helix, connector domain and heptad repeat 2 regions of the first betacoronavirus spike protein.

In one embodiment the amino acid sequence encoding the C terminal region is

(SEQ ID NO: 2)
CVNYDLYGITGQGIFKEVSAAYYNNWQNLLYDSNGNIIGFKDFLINKTYT
ILPCYSGRVSAAFYQNSSSPALLYRNLKCSYVLNNISFISQPFYFDSYLG
CVLNAVNLTSYSVSSCDLRMGSGFCIDYALPSSR [symbol] GISSPYRFVTFE
PFNVSFVNDSVETVGGLFEIQIPTNFTIAGHEEFIQTSSPKVTIDCSAFV
CSNYAACHDLLSEYGTFCDNINSILNEVNDLLDITQLQVANALMQGVTLS
SNLNTNLHSDVDNIDFKSLLGCLGSQCGSSSRS [symbol] LEDLLFNKVKLSDVGF
VEAYNNCTGGSEIRDLLCVQSFNGIKVLPPILSETQISGYTTAATVAAMF
PPWSAAAGVPF [symbol] LNVQYRINGLGVTMDVLNKNQKLIANAFNKALLSIQNG
FTAT [symbol] SALAKIQSVVNANAQALNSLLQQLFNKFGAISSSLQEILSRLD [symbol]
EAQVQIDRLINGRLTALNAYVSQQLSDITLIKAGASRAIEKVNECVKSQS
PRINFCGNGNHILSLVQNAPYGLLFIHFSYKPTSFKTVLVSPGLCLSGDR
GIAPKQGYFIKQNDSWMFTGSSYYYPEPISDKNVVFMNSCSVNFTKAPFI
YLNNSIPNLSDFEAELSLWFKNHTSIAPNLTENSHINATFLDLYYEMNVI
QESIKSLNSSFINLKEIGTYEM or a variant thereof.

In another embodiment, the amino acid sequence encoding the C terminal domain has at least 80% identity to the amino acid sequence of SEQ ID NO: 2.

In another embodiment, the amino acid sequence encoding the C terminal domain has at least 90% identity to the amino acid sequence of SEQ ID NO: 2.

In one embodiment, the amino acid sequence encoding the C terminal domain comprises one or more amino acid substitutions that increase protein yields, stability and/or structural confirmation of the protein.

In one embodiment, the one or more amino acid substitutions that substitutions that increase protein yields, stability and/or structural confirmation of the protein are selected from the group comprising the underlined residues in SEQ ID NO: 2;

CVNYDLYGITGQGIFKEVSAAYYNNWQNLLYDSNGNIIGFKDFLINKTYT
ILPCYSGRVSAAFYQNSSSPALLYRNLKCSYVLNNISFISQPFYFDSYLG
CVLNAVNLTSYSVSSCDLRMGSGFCIDYALPSSR [symbol] GISSPYRFVTFE
PFNVSFVNDSVETVGGLFEIQIPTNFTIAGHEEFIQTSSPKVTIDCSAFV
CSNYAACHDLLSEYGTFCDNINSILNEVNDLLDITQLQVANALMQGVTLS
SNLNTNLHSDVDNIDFKSLLGCLGSQCGSSSRS [symbol] LEDLLFNKVKLSDVGF
VEAYNNCTGGSEIRDLLCVQSFNGIKVLPPILSETQISGYTTAATVAAMF
PPWSAAAGVPF [symbol] LNVQYRINGLGVTMDVLNKNQKLIANAFNKALLSIQNG
FTAT [symbol] SALAKIQSVVNANAQALNSLLQQLFNKFGAISSSLQEILSRLD [symbol]
EAQVQIDRLINGRLTALNAYVSQQLSDITLIKAGASRAIEKVNECVKSQS
PRINFCGNGNHILSLVQNAPYGLLFIHFSYKPTSFKTVLVSPGLCLSGDR
GIAPKQGYFIKQNDSWMFTGSSYYYPEPISDKNVVFMNSCSVNFTKAPFI
YLNNSIPNLSDFEAELSLWFKNHTSIAPNLTENSHINATFLDLYYEMNVI
QESIKSLNSSFINLKEIGTYEM.

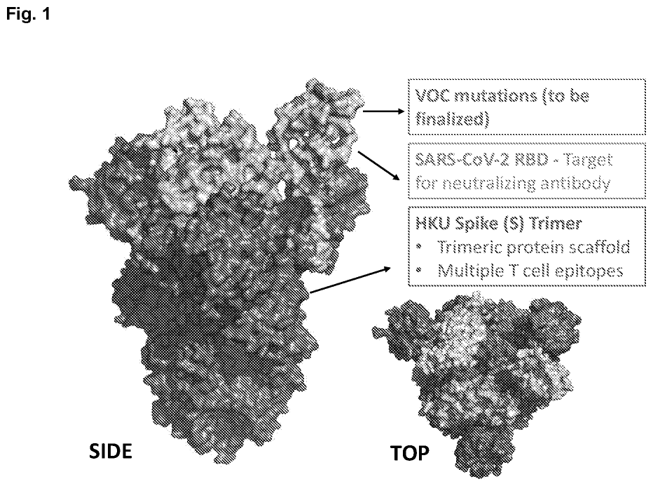

Eliciting neutralising antibodies that block trimeric Spike protein (FIG. 1) recognition of cellular receptor angiotensin converting enzyme 2 (ACE2) underpins the efficacy of first-generation COVID-19 vaccines. Key domains of the spike include the RBD (residues 319 to 591), which is the dominant target of the human neutralising antibody response, and contains sites bound by human antibodies with the greatest neutralisation potency and protective value in vivo. A minor fraction of neutralising antibodies targets the adjacent N-terminal domain (NTD) or the S2 domain.

Accordingly, the amino acid sequence encoding a receptor binding domain of the second betacoronavirus spike protein is from a betacoronavirus to which an immune response is to be elicited.

In one embodiment the receptor binding domain of the second betacoronavirus spike protein is a receptor binding domain comprising an amino acid sequence encoding a receptor binding domain of a betacoronavirus spike protein that has been modified to include an amino acid insertion, substitution or deletion of a second betacoronavirus to which an immune response is to be elicited.

As used herein, betacoronavius refers to the Beta genera of coronaviruses, and comprises four viral lineages. These four lineages include Severe acute respiratory syndrome-related coronavirus (SARSr-COV or SARS-COV), severe acute respiratory syndrome coronavirus 2 (SARS-COV-2), Middle East respiratory syndrome-related coronavirus (MERS-COV) Bovine coronavirus (BCoV), Human coronavirus OC43 (HCoV-OC43), Human coronavirus NL63 (HCoV-NL63), Human coronavirus 229 (HCoV-229), Rabbit coronavirus (RaCoV), Human coronavirus HKU1 (HKU1), Equine coronavirus (ECoV), Bovine coronavirus (BCoV), and Mouse hepatitis virus (MHV) among others.

In one embodiment, the first betacoronavirus spike protein is a HCoV-OC43, HCOVHKU1, HCOV-NL63 and HCoV-229, BCoV, RaCoV, MHV, ECOV betacoronavirus spike protein.

In one embodiment, the second betacoronavirus spike protein is a SARS-CoV, MERS-COV, or SARS-COV-2 betacoronavirus spike protein.

As used herein, “SARS-COV” refers to the Human coronavirus (strain SARS), and named by International Committee on Taxonomy of Viruses (ICTV) as HCoV-SARS. As used herein “SARS” refer to the disease caused by HCoV-SARS.

As used herein, “MERS-COV” refers to the Middle East respiratory syndrome-related coronavirus, and named by International Committee on Taxonomy of Viruses (ICTV) as MERS-COV. As used herein “MERS” refer to the disease caused by MERS-COV.

As used herein, “SARS-COV-2” refers to coronaviruses related to the Severe Acute Respiratory Syndrome (SARS) virus, and named by International Committee on Taxonomy of Viruses (ICTV) as severe acute respiratory syndrome coronavirus 2 (SARS-COV-2). As used herein “COVID-19” refer to the disease caused by SARS-COV-2.

### Mouse Hepatitis Virus Chimeric Polypeptides

The present inventors have demonstrated in Example 8 the construction of a Chimeric Trimeric Receptor Binding Domain polypeptide comprising a replacement of the RBD of a first betacoronavirus spike protein with the RBD of a second coronavirus RBD protein, and a trimerization sequence using a mouse hepatitis scaffold (e.g, wherein the first betacoronavirus is a mouse hepatitis virus).

Accordingly, in one embodiment, the present invention provides a chimeric polypeptide as described wherein the first betacoronavirus spike protein is a mouse hepatitis virus, wherein the amino acid sequence encoding the N terminal domain is

(SEQ ID NO: 18)
MLFVFILFLPSCLGYIGDFRCIQLVNSNGANVSAPSISTETVEVSQGLGT
YYVLDRVYLNATLLLTGYYPVDGSKFRNLALTGTNSVSLSWFQPPYLSQF
NDGIFAKVQNLKTSTPSGATAYFPTIVIGSLFGYTSYTVVIEPYNGVIMA
SVCQYTICQLPYTDCKPNTNGNKLIGFWHTDVKPPICVLKRNFTLNVNAD
AFYFHFYQHGGTFYAYYADKPSATTFLFSVYIGDILTQYYVLPFICNPTA
GSTFAPRYWVTPLVKRQYLFNFNQKGVITSAVDCASSYTSEIKCKTQSML
PSTGVYELSGYTV or a variant thereof

In another embodiment, the amino acid sequence encoding the N terminal domain has at least 80% identity to the amino acid sequence of SEQ ID NO: 18.

In another embodiment, the amino acid sequence encoding the N terminal domain has at least 90% identity to the amino acid sequence of SEQ ID NO: 18.

the present invention provides a chimeric polypeptide as described wherein the first betacoronavirus spike protein is a mouse hepatitis virus, wherein the amino acid sequence the C terminal is encoding region

(SEQ ID NO: 19)
CVKYDLYGITGQGVFKEVKADYYNSWQTLLYDVNGNLNGFRDLTTNKTYT
IRSCYSGRVSAAFHKDAPEPALLYRNINCSYVFSNNISREENPLNYFDSY
LGCVVNADNRTDEALPNCDLRMGAGLCVDYSKSSR [symbol] GSVSTGYRLTTFE
PYTPMLVNDSVQSVDGLYEMQIPTNFTIGHHEEFIQTRSPKVTIDCAAFV
CGDNTACRQQLVEYGSFCVNVNAILNEVNNLLDNMQLQVASALMQGVTIS
SRLPDGISGPIDDINFSPLLGCIGSTCAEDGNGPSAIRGRS [symbol] IEDLLFDK
VKLSDVGFVEAYNNCTGGQEVRDLLCVQSFNGIKVLPPVLSESQISGYTT
GATAAAMFPPWSAAAGVPF [symbol] LSVQYRINGLGVTMNVLSENQKMIASAFNN
ALGAIQDGFDAT [symbol] SALGKIQSVVNANAEALNNLLNQLSNRFGAISASLQE
ILTRLE [symbol] [symbol] EAKAQIDRLINGRLTALNAYISKQLSDSTLIKVSAAQAIEKV
NECVKSQTTRINFCGNGNHILSLVQNAPYGLYFIHFSYVPISFTTANVSP
GLCISGDRGLAPKAGYFVQDDGEWKFTGSSYYYPEPITDKNSVIMSSCAV
NYTKAPEVFLNTSIPNPPDFKEELDKWFKNQTSIAPDLSLDFEKLNVTLL
DLTYEMNRIQDAIKKLNESYINLKEVGTYEMY or a variant
thereof.

In another embodiment, the amino acid sequence encoding the C terminal domain has at least 80% identity to the amino acid sequence of SEQ ID NO: 19.

In another embodiment, the amino acid sequence encoding the C terminal domain has at least 90% identity to the amino acid sequence of SEQ ID NO: 19.

In one embodiment, the one or more amino acid substitutions that substitutions that increase protein yields, stability and/or structural confirmation of the protein are selected from the group comprising the underlined residues in SEQ ID NO: 19;

(SEQ ID NO: 19)
CVKYDLYGITGQGVFKEVKADYYNSWQTLLYDVNGNLNGFRDLTTNKTYT
IRSCYSGRVSAAFHKDAPEPALLYRNINCSYVFSNNISREENPLNYFDSY
LGCVVNADNRTDEALPNCDLRMGAGLCVDYSKSSR [symbol] GSVSTGYRLTTFE
PYTPMLVNDSVQSVDGLYEMQIPTNFTIGHHEEFIQTRSPKVTIDCAAFV
CGDNTACRQQLVEYGSFCVNVNAILNEVNNLLDNMQLQVASALMQGVTIS
SRLPDGISGPIDDINFSPLLGCIGSTCAEDGNGPSAIRGRS [symbol] IEDLLFDK
VKLSDVGFVEAYNNCTGGQEVRDLLCVQSFNGIKVLPPVLSESQISGYTT
GATAAAMFPPWSAAAGVPF [symbol] LSVQYRINGLGVTMNVLSENQKMIASAFNN
ALGAIQDGFDATPSALGKIQSVVNANAEALNNLLNQLSNRFGAISASLQE
ILTRLE [symbol] [symbol] EAKAQIDRLINGRLTALNAYISKQLSDSTLIKVSAAQAIEKV
NECVKSQTTRINFCGNGNHILSLVQNAPYGLYFIHFSYVPISFTTANVSP
GLCISGDRGLAPKAGYFVQDDGEWKFTGSSYYYPEPITDKNSVIMSSCAV
NYTKAPEVFLNTSIPNPPDFKEELDKWFKNQTSIAPDLSLDFEKLNVTLL
DLTYEMNRIQDAIKKLNESYINLKEVGTYEMY or a variant
thereof

### Equine Coronavirus Chimeric Polypeptides

The present inventors have demonstrated in Example 8 the construction of a Chimeric Trimeric Receptor Binding Domain polypeptide comprising a replacement of the RBD of a first betacoronavirus spike protein with the RBD of a second coronavirus RBD protein, and a trimerization sequence using an equine coronavirus scaffold (e.g, wherein the first betacoronavirus is an equine coronavirus).

In one embodiment, the present invention provides a chimeric polypeptide as described wherein the first betacoronavirus spike protein is an equine coronavirus, wherein the amino acid sequence encoding the N terminal domain is

(SEQ ID NO: 20)
MFLILLISLPTAFAVIGDLKCTTVSINDVDTGVPSISTDTVDVTNGLGTY
YVLDRVYLNTTLLLNGYYPTSGSTYRNMALKGTLLLSTLWFKPPFLSDFT
NGIFAKVKNTKVIKDGVMYSEFPAITIGSTFVNTSYSVVVQPHTTILGNK
LQGFLEISVCQYTMCEYPNTICNPNLGNQRVELWHWDTGVVSCLYKRNFT
YDVNADYLYFHFYQEGGTFYAYFTDTGVVTKFLFNVYLGTVLSHYYVMPL
TCNSALTLEYWVTPLTSKQYLLAFNQDGVIFNAVDCKSDFMSEIKCKTLS
IAPSTGVYELNGYTV or a variant thereof

In another embodiment, the amino acid sequence encoding the N terminal domain has at least 80% identity to the amino acid sequence of SEQ ID NO: 20.

In another embodiment, the amino acid sequence encoding the N terminal domain has at least 90% identity to the amino acid sequence of SEQ ID NO: 20.

the present invention provides a chimeric polypeptide as described wherein the first betacoronavirus spike protein is an equine coronavirus, wherein the amino acid sequence encoding the terminal region is

(SEQ ID NO: 21)
CVNYDLYGITGQGIFVEVNATYYNSWQNLLYDSNGNLYGFRDYLTNRTFM
IRSCYSGRVSAAFHANSSEPALLFRNIKCNYVFNNTLSRQLQPINYFDSY
LGCVVNADNSTSSVVQTCDLTVGSGYCVDYSTKSR [symbol] GSITTGYRFTNFE
PFTVNSVNDSLEPVGGLYEIQIPSEFTIGNMEEFIQTSSPKVTIDCSAFV
CGDYAACKSQLVEYGSFCDNINAILTEVNELLDTTQLQVANSLMNGVTLS
TKLKDGVNFNVDDINFSPVLGCLGSDCNKVSSRS [symbol] IEDLLFSKVKLSDVG
FVEAYNNCTGGAEIRDLICVQSYNGIKVLPPLLSENQISGYTLAATSASL
FPPWSAAAGVPF [symbol] LNVQYRINGIGVTMDVLSQNQKLIANAFNNALGAIQE
GFDAT [symbol] SALVKIQAVVNANAEALNNLLQQLSNRFGAISSSLQEILSRLD [symbol]
EAQAQIDRLINGRLTALNAYVSQQLSDSTLVKFSAAQAMEKVNECVKS
QSSRINFCGNGNHIISLVQNAPYGLYFIHFSYVPTKYVTAKVSPGLCIAG
DRGIAPKSGYFVNVNNTWMFTGSGYYYPEPITGNNVVVMSTCAVNYTKAP
DVMLNISTPNLPYFKEELDQWFKNQTSVAPDLSLDYINVTFLDLQDEMNR
LQEAIKVLNQSYINLKDIGTYEYY or a variant thereof.

In another embodiment, the amino acid sequence encoding the C terminal domain has at least 80% identity to the amino acid sequence of SEQ ID NO: 21.

In another embodiment, the amino acid sequence encoding the C terminal domain has at least 90% identity to the amino acid sequence of SEQ ID NO: 21.

In one embodiment, the one or more amino acid substitutions that substitutions that increase protein yields, stability and/or structural confirmation of the protein are selected from the group comprising the underlined residues in SEQ ID NO: 21;

(SEQ ID NO: 21)
CVNYDLYGITGQGIFVEVNATYYNSWQNLLYDSNGNLYGFRDYLTNRTFM
IRSCYSGRVSAAFHANSSEPALLFRNIKCNYVFNNTLSRQLQPINYFDSY
LGCVVNADNSTSSVVQTCDLTVGSGYCVDYSTKSR [symbol] GSITTGYRFTNFE
PFTVNSVNDSLEPVGGLYEIQIPSEFTIGNMEEFIQTSSPKVTIDCSAFV
CGDYAACKSQLVEYGSFCDNINAILTEVNELLDTTQLQVANSLMNGVTLS
TKLKDGVNFNVDDINFSPVLGCLGSDCNKVSSRS [symbol] IEDLLFSKVKLSDVG
FVEAYNNCTGGAEIRDLICVQSYNGIKVLPPLLSENQISGYTLAATSASL
FPPWSAAAGVPF [symbol] LNVQYRINGIGVTMDVLSQNQKLIANAFNNALGAIQE
GFDAT [symbol] SALVKIQAVVNANAEALNNLLQQLSNRFGAISSSLQEILSRLD [symbol]
EAQAQIDRLINGRLTALNAYVSQQLSDSTLVKFSAAQAMEKVNECVKS
QSSRINFCGNGNHIISLVQNAPYGLYFIHFSYVPTKYVTAKVSPGLCIAG
DRGIAPKSGYFVNVNNTWMFTGSGYYYPEPITGNNVVVMSTCAVNYTKAP
DVMLNISTPNLPYFKEELDQWFKNQTSVAPDLSLDYINVTFLDLQDEMNR
LQEAIKVLNQSYINLKDIGTYEYY or a variant thereof.

The emergence of multiple variants of concern (VOC) bearing convergent RBD mutations shows SARS-COV-2 is adapting to human hosts. Without wishing to be bound by theory, the present inventors that multimerization of RBD antigens from a second coronavirus in the context of whole spike using the N-terminal and C-terminal regions of a first betacoronavirus spike protein allows the refocusing of immunity onto critical RBD changes, while minimising non- or weakly neutralising responses (such as those targeting the NTD or S2). The present inventors have demonstrated [Tan, H. X. et al. (2021) Nat Commun 12 (1), 1403] monomeric RBD to poorly be immunogenic in mice and non-human primates models compared to trimeric spike vaccines. The present inventors propose that multimerisation of RBD antigens in the context of whole Spike is important to maintain potent immunogenicity and/or to avoid immune responses to regions of spike not exposed to the immune system during pathogenic betacoronavirus infection.

The present inventors have demonstrated in Example 8 the construction and expression of a stable Chimeric Trimeric Receptor Binding Domain polypeptide comprising a replacement of the RBD of a first betacoronavirus spike protein with the RBD of a second coronavirus RBD protein, and a trimerization sequence using an Omicron RBD.

Accordingly, in another embodiment, the second betacoronavirus spike protein is selected from the group consisting of a B.1.1.7 and Q (Alpha), B.1.351 (Beta), B.1.617.2 and AY (Delta), P.1 (Gamma), B.1.427 (Epsilon), B.1.429 (Epsilon), B.1.525 (Eta), B.1.526 (Iota) B.1.617.1 (Kappa), B.1.617.3, C.37 (Lambda), B.1.621 (Mu), B.1.1.529 (Omicron), and P.2 (Zeta) spike protein.

In a further embodiment, the present invention provides a chimeric polypeptide as described herein, wherein the second betacoronavirus spike protein is selected from the group consisting of a B.1.1.529, BA.1, BA.1.1, BA.2, BA.3, BA.4 and BA.5 spike protein.

SARS-COV-2 isolate sequences are available via the Global Initiative on Sharing All Influenza Data (GISAID) database.

Previous studies have characterised escape mutations—mutations that emerge in virus populations exposed to either mAbs or convalescent plasma containing polyclonal antibodies—have characterised that substitution or deletion of residue 140, or substitutions of residues 148, 140, 150, 151, 339, 345, 346, 352, 371, 373, 375, 376, 378, 405, 406, 408, 417, 439, 440, 441, 443, 444, 445, 446, 447, 448, 449, 450, 452, 453, 455, 456, 458, 460, 472, 473, 474, 476, 477, 478, 479, 483, 484, 485, 486, 487, 489, 490, 493, 494, 496, 498, 499, 501, 503, 504, 505 and 519 of the spike RBD has an effect on mAb binding and/or polyclonal sera binding, and/or emergence of escape mutants to mAbs and/or polyclonal sera binding.

In one embodiment, the RBD domain is an RBD domain of a variant selected from the group consisting of B.1.1.7 and Q (Alpha), B.1.351 (Beta), B.1.617.2 and AY (Delta), P.1 (Gamma), B.1.427 (Epsilon), B.1.429 (Epsilon), B.1.525 (Eta), B.1.526 (Iota) B.1.617.1 (Kappa), B.1.617.3, C.37 (Lambda), B.1.621 (Mu), B.1.1.529 (Omicron), and P.2 (Zeta) spike protein.

In a further embodiment, the RBD domain is an RBD domain of a variant selected from the group consisting of a B.1.1.529, BA.1, BA.1.1, BA.2, BA.3, BA.4 and BA.5 spike protein.

In one embodiment wherein the amino acid sequence encoding the one or more immunogenic epitope is a receptor binding domain of the first betacoronavirus spike protein comprising one or more amino acid variant of a second betacoronavirus spike protein, the one or more amino acid variant is selected from an amino acid insertion, deletion or an amino acid substitution.

In another embodiment wherein the amino acid sequence encoding the one or more immunogenic epitope is a receptor binding domain of the first betacoronavirus spike protein comprising one or more amino acid variant of a second betacoronavirus spike protein, wherein the second betacoronavirus spike protein is selected from the group consisting of a B.1.1.7 and Q (Alpha), B.1.351 (Beta), B.1.617.2 and AY (Delta), P.1 (Gamma), B.1.427 (Epsilon), B.1.429 (Epsilon), B.1.525 (Eta), B.1.526 (Iota) B.1.617.1 (Kappa), B.1.617.3, C.37 (Lambda), B.1.621 (Mu), B.1.1.529 (Omicron), and P.2 (Zeta) spike protein.

SARS-COV-2 variants include:
a. B.1.1.7 (Alpha), which has the following Spike Protein Substitutions: 69del, 70del, 144del, (E484K*), (S494P*), N501Y, A570D, D614G, P681H, T7161, S982A, D1118H (K1191N*).
b. B.1.351 (Beta), which has the following Spike Protein Substitutions: D80A, D215G, 241del, 242del, 243del, K417N, E484K, N501Y, D614G, A701V.
c. B.1.617.2 (Delta), which has the following Spike Protein Substitutions: T19R, (V70F*), T951, G142D, E156-, F157-, R158G, (A222V*), (W258L*), (K417N*), L452R, T478K, D614G, P681R, D950N.
d. P.1 (Gamma), which has the following Spike Protein Substitutions: L18F, T20N, P26S, D138Y, R190S, K417T, E484K, N501Y, D614G, H655Y, T1027I.
e. B.1.427 (Epsilon), which has the following Spike Protein Substitutions: L452R, D614G f. B.1.429 (Epsilon), which has the following Spike Protein Substitutions: S13I, W152C, L452R, D614G.
g. B.1.525 (Eta), which has the following Spike Protein Substitutions: A67V, 69del, 70del, 144del, E484K, D614G, Q677H, F888L.
h. B.1.526 (Iota) which has the following Spike Protein Substitutions: L5F, (D80G*), T951, (Y144-*), (F157S*), D253G, (L452R*), (S477N*), E484K, D614G, A701V, (T859N*), (D950H*), (Q957R*).
i. B.1.617.1 (Kappa), which has the following Spike Protein Substitutions: (T951), G142D, E154K, L452R, E484Q, D614G, P681R, Q1071H.
j. B.1.617.3, which has the following Spike Protein Substitutions: T19R, G142D, L452R, E484Q, D614G, P681R, D950N.
k. C.37 (Lambda) which has the following Spike Protein Substitutions: G75V, T76I, del247/253, L452Q, F490S, D614G and T859N.
l. B.1.621 (Mu) which has the following Spike Protein Substitutions: T951, Y144S, Y145N, R346K, E484K, N501Y, D614G, P681H, and D950N.
m. B.1.1.529 (Omicron BA.1), which has the following Spike Protein Substitutions: G339D, S371L, S373P, S375F, K417N, N440K, G446S, S477N, T478K, E484A, Q493R, G496S, Q498R, N501Y, Y505H.
n. B.1.1.529 (Omicron BA.2), which has the following Spike Protein Substitutions: G339D, S371F, S373P, S375F, T376A, D405N, R408S, K417N, N440K, S477N, T478K, E484A, Q493R, Q498R, N501Y, Y505H.
o. B.1.1.529 (Omicron BA.3), which has the following Spike Protein Substitutions: G339D, S371F, S373P, S375F, D405N, K417N, N440K, G446S, S477N, T478K, E484A, Q493R, Q498R, N501Y, Y505H.
p. B.1.1.529 (Omicron BA.4), which has the following Spike Protein Substitutions: G339D, S371F, S373P, S375F, T376A, D405N, R408S, K417N, N440K, L452R, S477N, T478K, E484A, F486V; R493Q, Q498R, N501Y, Y505H.
q. B.1.1.529 (Omicron BA.5), which has the following Spike Protein Substitutions: G339D, S371F, S373P, S375F, T376A, D405N, R408S, K417N, N440K, L452R, S477N, T478K, E484A, F486V; R493Q, Q498R, N501Y, Y505H.
r. B.1.1.529 (Omicron BA.2.75), which has the following Spike Protein Substitutions: G339H, S371F, S373P, S375F, T376A, D405N, R408S, K417N, N440K, G446S, N460K, S477N, T478K, E484A, Q493, Q498R, N501Y, Y505H.
   * denotes a variation detected in some but not all sequences of a particular VOC.

In one embodiment the one or more amino acid variant is selected from one or more amino acid variant of RBD variants of the following groups:
a. 69del, 70del, 144del, (E484K*), (S494P*), N501Y, A570D, D614G, P681H, T7161, S982A, D1118H, and (K1191N*);
b. D80A, D215G, 241del, 242del, 243del, K417N, E484K, N501Y, D614G, and A701V;
c. T19R, (V70F*), T951, G142D, E156-, F157-, R158G, (A222V*), (W258L*), (K417N*), L452R, T478K, D614G, P681R, and D950N;
d. L18F, T20N, P26S, D138Y, R190S, K417T, E484K, N501Y, D614G, H655Y, and T1027I;
e. L452R, and D614G;
f. S13I, W152C, L452R, and D614G;
g. A67V, 69del, 70del, 144del, E484K, D614G, Q677H, and F888L;
h. L5F, (D80G*), T951, (Y144-*), (F157S*), D253G, (L452R*), (S477N*), E484K, D614G, A701V, (T859N*), (D950H*), and (Q957R*);
i. (T951), G142D, E154K, L452R, E484Q, D614G, P681R, and Q1071H;
j. T19R, G142D, L452R, E484Q, D614G, P681R, and D950N;
k. G75V, T76I, del247/253, L452Q, F490S, D614G and T859N;
l. T951, Y144S, Y145N, R346K, E484K, N501Y, D614G, P681H, and D950N.
m. G339D, S371L, S373P, S375F, K417N, N440K, G446S, S477N, T478K, E484A, Q493R, G496S, Q498R, N501Y, Y505H.
n. G339D, S371F, S373P, S375F, T376A, D405N, R408S, K417N, N440K, S477N, T478K, E484A, Q493R, Q498R, N501Y, Y505H.
o. G339D, S371F, S373P, S375F, D405N, K417N, N440K, G446S, S477N, T478K, E484A, Q493R, Q498R, N501Y, Y505H.
p. G339D, S371F, S373P, S375F, T376A, D405N, R408S, K417N, N440K, L452R, S477N, T478K, E484A, F486V; R493Q, Q498R, N501Y, Y505H.
q. G339D, S371F, S373P, S375F, T376A, D405N, R408S, K417N, N440K, L452R, S477N, T478K, E484A, F486V; R493Q, Q498R, N501Y, Y505H.
r. G339H, S371F, S373P, S375F, T376A, D405N, R408S, K417N, N440K, G446S, N460K, S477N, T478K, E484A, Q493, Q498R, N501Y, Y505H.
   or a combination thereof. * denotes a variation detected in some but not all sequences of a particular VOC.

The chimeric polypeptides and chimeric trimeric RBD vaccines described erein provide a platform based upon a pre-fusion stabilised trimeric spike scaffold derived from first betacoronavirus (e.g. endemic human coronavirus HKU1), into which the heterologous RBD of SARS-COV-2 has been engineered. The SARS-COV-2 RBD will be modified to include mutations from VOC that evade current vaccine candidates, and to be continually updated in the face of new emerging strains including emerging VOC. Accordingly, in one embodiment, the RBD domain is an RBD domain of an artificial variant generated by combining one or more spike protein substitutions of more or more known VOC. In another embodiment, the RBD domain is an RBD domain of an artificial variant generated by combining one or more spike protein substitutions of predicted VOC.

For example, in one embodiment, the RBD domain is an RBD domain of an of a first betacoronavirus spike protein comprising one or more immunogenic epitopes of a second betacoronavirus spike protein.

In another embodiment, the RBD domain is an RBD domain of an of a first betacoronavirus spike protein comprising one or more amino acid variant of a second betacoronavirus spike protein.

In another embodiment, the RBD domain is an RBD domain of an of a first betacoronavirus spike protein comprising one or more amino acid variant of a second betacoronavirus spike protein, wherein the amino acid variant is selected from an amino acid insertion, deletion or an amino acid substitution.

The present inventors propose that the trimeric spike scaffold of the chimeric polypeptides described herein ensures RBD multimerisation while simultaneously ameliorating the risk of immune imprinting due to recall of non-neutralising SARS-COV-2 spike-specific immunity. The presentation of SARS-COV-2 RBD in a pre-fusion stabilised, trimeric form maintains immunogenicity and drives focused re-targeting of neutralising antibodies onto the heterologous (e.g. VOC) RBD, and the heterologous betacoronavirus scaffold (e.g. HKU1-based scaffold) minimises boosting of the potent (non-neutralising) immune memory established by prior COVID-19 or SARS-COV-2 vaccines.

In one embodiment, the first betacoronavirus spike protein is a spike protein into which a heterologous RBD (e.g. from a second betacoronavirus) can be engineered and be antigenically intact.

In one embodiment, the first betacoronavirus spike protein is a spike protein into which a heterologous immunogenic epitope (e.g. from a second betacoronavirus) can be engineered and be antigenically intact.

In one embodiment, the first betacoronavirus spike protein is a HCoV-OC43, HCoVHKU1, HCOV-NL63 and HCoV-229, BCOV, RaCoV, MHV, ECOV betacoronavirus spike protein.

In another embodiment the first betacoronavirus spike protein is a spike protein of an endemic human betacoronavirus.

The present inventors have demonstrated (data not shown) that HKU1 memory responses while widespread are comparably weak in nature. The present inventors propose that using a HKU1-based scaffold may facilitate recruitment of widespread CD4 helper T cell responses for optimal antibody elicitation.

In a preferred embodiment, the first betacoronavirus spike protein is a HCoVHKU1 betacoronavirus spike protein.

The present inventors have demonstrated that the trimeric spike scaffold (derived from a human common cold coronavirus) ensures RBD multimerisation while simultaneously ameliorating the risk of immune imprinting due to recall of non-neutralising SARS-COV-2 spike-specific immunity.

The present inventors propose that the chimeric trimeric RBD vaccines described herein comprising the chimeric polypeptides described herein present the RBD in an antigenically intact manner, in a native format. For example, non-trimeric RBD vaccines will present parts of the RBD that are normally hidden and which are likely to become immunogenic once immunised into a subject. Without wishing to be bound by theory, the present inventors propose the RBD of the chimeric trimeric RBD vaccines is presented in the same trimeric context as during infection with a betacoronavirus.

In a further embodiment, the chimeric polypeptide further comprises an amino acid sequence encoding a trimerization domain.

Suitable trimerization domains invention include T4 fibritin foldon (PDB ID: 4NCV) and viral capsid protein SHP (PDB: 1TD0).

In a preferred embodiment the trimerization domain is the C-terminal domain of T4 fibritin.

In some embodiments, the trimerization domain is linked to a tag via short GS linker, for example, 1-6 tandem repeats of GS.

In some embodiments, an amino acid-tag can be added to C-terminal of the trimerization motif to facilitate protein purification.

In one embodiment, the amino acid sequence encoding the RBD domain is

(SEQ ID NO: 3)
QPTESIVRFPNITNLCPFGEVFNATRFASVYAWNRKRISNCVADYSVLYN
SASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDIST
EIYQAGSTPCNGVEGENCYFPLQSYGFQPTNGVGYQPYRVVVLSFELLHA
PATVCGPKKSTNLVKNK or a variant thereof.

In another embodiment, the amino acid sequence encoding the RBD domain has at least 80% identity to the amino acid sequence of SEQ ID NO: 3.

In another embodiment, the amino acid sequence encoding the N terminal domain has at least 90% identity to the amino acid sequence of SEQ ID NO: 3.

In one embodiment, the amino acid sequence encoding the RBD domain encodes a B.1.1.7 (Alpha) RBD domain.

In one embodiment, the amino acid sequence encoding the RBD domain is

(SEQ ID NO: 4)
QPTESIVRFPNITNLCPFGEVFNATRFASVYAWNRKRISNCVADYSVLYN
SASFSTFKCYGVSPTKLNDLCFTNVYADSFVIRGDEVRQIAPGQTGNIAD
YNYKLPDDFTGCVIAWNSNNLDSKVGGNYNYLYRLFRKSNLKPFERDIST
EIYQAGSTPCNGVEGENCYFPLQSYGFQPTYGVGYQPYRVVVLSFELLHA
PATVCGPKKSTNLVKNK or a variant thereof.

In another embodiment, the amino acid sequence encoding the N terminal domain has at least 80% identity to the amino acid sequence of SEQ ID NO: 4.

In another embodiment, the amino acid sequence encoding the RBD domain has at least 90% identity to the amino acid sequence of SEQ ID NO: 4.

In one embodiment, the amino acid sequence encoding the RBD domain encodes a B.1.351 (Beta) RBD domain.

In one embodiment, the amino acid sequence encoding the RBD domain is

(SEQ ID NO: 5)
QPTESIVRFPNITNLCPFGEVFNATRFASVYAWNRKRISNCVADYSVLYN
SASFSTFKCYGVSPTKLNDLCFTNVYADSFVIRGDEVRQIAPGQTGNIAD
YNYKLPDDFTGCVIAWNSNNLDSKVGGNYNYLYRLFRKSNLKPFERDIST
EIYQAGSTPCNGVKGFNCYFPLQSYGFQPTYGVGYQPYRVVVLSFELLHA
PATVCGPKKSTNLVKNK or a variant thereof.

In another embodiment, the amino acid sequence encoding the RBD domain has at least 80% identity to the amino acid sequence of SEQ ID NO: 5.

In another embodiment, the amino acid sequence encoding the RBD domain has at least 90% identity to the amino acid sequence of SEQ ID NO: 5.

In one embodiment, the amino acid sequence encoding the RBD domain encodes a P.1 (Gamma) RBD domain.

In one embodiment, the amino acid sequence encoding the RBD domain is

(SEQ ID NO: 6)
QPTESIVRFPNITNLCPFGEVFNATRFASVYAWNRKRISNCVADYSVLYN
SASFSTFKCYGVSPTKLNDLCFTNVYADSFVIRGDEVRQIAPGQTGTIAD
YNYKLPDDFTGCVIAWNSNNLDSKVGGNYNYLYRLFRKSNLKPFERDIST
EIYQAGSTPCNGVKGFNCYFPLQSYGFQPTYGVGYQPYRVVVLSFELLHA
PATVCGPKKSTNLVKNK, or a variant thereof.

In another embodiment, the amino acid sequence encoding the RBD domain has at least 80% identity to the amino acid sequence of SEQ ID NO: 6.

In another embodiment, the amino acid sequence encoding the RBD domain has at least 90% identity to the amino acid sequence of SEQ ID NO: 6.

In one embodiment, the amino acid sequence encoding the RBD domain encodes a B.1.617.2 (Delta) RBD domain.

In one embodiment, the amino acid sequence encoding the RBD domain is

(SEQ ID NO: 7)
QPTESIVRFPNITNLCPFGEVFNATRFASVYAWNRKRISNCVADYSVLYN
SASFSTFKCYGVSPTKLNDLCFTNVYADSFVIRGDEVRQIAPGQTGKIAD
YNYKLPDDFTGCVIAWNSNNLDSKVGGNYNYRYRLFRKSNLKPFERDIST
EIYQAGSKPCNGVEGENCYFPLQSYGFQPTNGVGYQPYRVVVLSFELLHA
PATVCGPKKSTNLVKNK
or
a variant thereof.

In another embodiment, the amino acid sequence encoding the RBD domain has at least 80% identity to the amino acid sequence of SEQ ID NO: 7.

In another embodiment, the amino acid sequence encoding the RBD domain has at least 90% identity to the amino acid sequence of SEQ ID NO: 7.

In one embodiment, the amino acid sequence encoding the RBD domain encodes a B.1.525 (Eta)/B.1.526 (Iota) RBD domain.

In one embodiment, the amino acid sequence encoding the RBD domain is

(SEQ ID NO: 8)
QPTESIVRFPNITNLCPFGEVFNATRFASVYAWNRKRISNCVADYSVLYN
SASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDIST
EIYQAGSTPCNGVKGFNCYFPLQSYGFQPTNGVGYQPYRVVVLSFELLHA
PATVCGPKKSTNLVKNK
or
a variant thereof.

In another embodiment, the amino acid sequence encoding the RBD domain has at least 80% identity to the amino acid sequence of SEQ ID NO: 8.

In another embodiment, the amino acid sequence encoding the RBD domain has at least 90% identity to the amino acid sequence of SEQ ID NO: 8.

In one embodiment, the amino acid sequence encoding the RBD domain encodes a B.1.617.1 (Kappa)/1.617.3 RBD domain.

In one embodiment, the amino acid sequence encoding the RBD domain is

(SEQ ID NO: 9)
QPTESIVRFPNITNLCPFGEVFNATRFASVYAWNRKRISNCVADYSVLYN
SASFSTFKCYGVSPTKLNDLCFTNVYADSFVIRGDEVRQIAPGQTGKIAD
YNYKLPDDFTGCVIAWNSNNLDSKVGGNYNYRYRLFRKSNLKPFERDIST
EIYQAGSTPCNGVQGFNCYFPLQSYGFQPTNGVGYQPYRVVVLSFELLHA
PATVCGPKKSTNLVKNK
or
a variant thereof.

In another embodiment, the amino acid sequence encoding the RBD domain has at least 80% identity to the amino acid sequence of SEQ ID NO: 9.

In another embodiment, the amino acid sequence encoding the RBD domain has at least 90% identity to the amino acid sequence of SEQ ID NO: 9.

In one embodiment, the amino acid sequence encoding the RBD domain encodes a C.37 (Lambda) RBD domain.

In one embodiment, the amino acid sequence encoding the RBD domain is

(SEQ ID NO: 10)
QPTESIVRFPNITNLCPFGEVFNATRFASVYAWNRKRISNCVADYSVLYN
SASFSTFKCYGVSPTKLNDLCFTNVYADSFVIRGDEVRQIAPGQTGKIAD
YNYKLPDDFTGCVIAWNSNNLDSKVGGNYNYQYRLFRKSNLKPFERDIST
EIYQAGSTPCNGVEGFNCYSPLQSYGFQPTNGVGYQPYRVVVLSFELLHA
PATVCGPKKSTNLVKNK
or
a variant thereof.

In another embodiment, the amino acid sequence encoding the RBD domain has at least 80% identity to the amino acid sequence of SEQ ID NO: 10.

In another embodiment, the amino acid sequence encoding the RBD domain has at least 90% identity to the amino acid sequence of SEQ ID NO: 10.

In one embodiment, the amino acid sequence encoding the RBD domain encodes a Composite 5MUT 1.0 RBD domain.

In one embodiment, the amino acid sequence encoding the RBD domain is

(SEQ ID NO: 11)
QPTESIVRFPNITNLCPFGEVFNATRFASVYAWNRKRISNCVADYSVLYN
SASFSTFKCYGVSPTKLNDLCFTNVYADSFVIRGDEVRQIAPGQTGNIAD
YNYKLPDDFTGCVIAWNSNNLDSKVGGNYNYRYRLFRKSNLKPFERDIST
EIYQAGSKPCNGVKGFNCYFPLQSYGFQPTYGVGYQPYRVVVLSFELLHA
PATVCGPKKSTNLVKNK
or
a variant thereof.

In another embodiment, the amino acid sequence encoding the RBD domain has at least 80% identity to the amino acid sequence of SEQ ID NO: 11.

In another embodiment, the amino acid sequence encoding the RBD domain has at least 90% identity to the amino acid sequence of SEQ ID NO: 11.

In one embodiment, the amino acid sequence encoding the RBD domain encodes a Composite 5MUT 2.0 RBD domain.

In one embodiment, the amino acid sequence encoding the RBD domain is

(SEQ ID NO: 12)
QPTESIVRFPNITNLCPFGEVFNATRFASVYAWNRKRISNCVADYSVLYN
SASFSTFKCYGVSPTKLNDLCFTNVYADSFVIRGDEVRQIAPGQTGNIAD
YNYKLPDDFTGCVIAWNSNNLDSKVGGNYNYRYRLFRKSNLKPFERDIST
EIYQAGNTPCNGVKGFNCYFPLQSYGFQPTYGVGYQPYRVVVLSFELLHA
PATVCGPKKSTNLVKNK
or
a variant thereof.

In another embodiment, the amino acid sequence encoding the RBD domain has at least 80% identity to the amino acid sequence of SEQ ID NO: 12.

In another embodiment, the amino acid sequence encoding the RBD domain has at least 90% identity to the amino acid sequence of SEQ ID NO: 12.

In one embodiment, the amino acid sequence encoding the RBD domain encodes an Omicron BA.1 RBD domain.

In one embodiment, the amino acid sequence encoding the Omicron BA. 1 RBD domain is

(SEQ ID NO: 22)
QPTESIVRFPNITNLCPFDEVFNATRFASVYAWNRKRISNCVADYSVLYN
LAPFFTFKCYGVSPTKLNDLCFTNVYADSFVIRGDEVRQIAPGQTGNIAD
YNYKLPDDFTGCVIAWNSNKLDSKVSGNYNYLYRLFRKSNLKPFERDIST
EIYQAGNKPCNGVAGFNCYFPLRSYSFRPTYGVGHQPYRVVVLSFELLHA
PATVCGPKKSTNLVKNK
or
a variant thereof.

In another embodiment, the amino acid sequence encoding the RBD domain has at least 80% identity to the amino acid sequence of SEQ ID NO: 22.

In another embodiment, the amino acid sequence encoding the RBD domain has at least 90% identity to the amino acid sequence of SEQ ID NO: 22.

In one embodiment, the amino acid sequence encoding the RBD domain encodes an Omicron BA.2 RBD domain.

In one embodiment, the amino acid sequence encoding the Omicron BA.2 RBD domain is

(SEQ ID NO: 23)
QPTESIVRFPNITNLCPFDEVFNATRFASVYAWNRKRISNCVADYSVLYN
FAPFFAFKCYGVSPTKLNDLCFTNVYADSFVIRGNEVSQIAPGQTGNIAD
YNYKLPDDFTGCVIAWNSNKLDSKVGGNYNYLYRLFRKSNLKPFERDIST
EIYQAGNKPCNGVAGFNCYFPLRSYGFRPTYGVGHQPYRVVVLSFELLHA
PATVCGPKKSTNLVKNK
or
a variant thereof.

In another embodiment, the amino acid sequence encoding the Omicron BA.2 RBD domain has at least 80% identity to the amino acid sequence of SEQ ID NO: 23.

In another embodiment, the amino acid sequence encoding the Omicron BA.2 RBD domain has at least 90% identity to the amino acid sequence of SEQ ID NO: 23.

In one embodiment, the amino acid sequence encoding a trimerization domain is GYIPEAPRDGQAYVRKDGEWVLLSTFL (SEQ ID NO: 13) or a variant thereof.

In another embodiment, the amino acid sequence encoding the trimerization domain has at least 80% identity to the amino acid sequence of SEQ ID NO: 13.

In another embodiment, the amino acid sequence encoding the trimerisation domain has at least 90% identity to the amino acid sequence of SEQ ID NO: 13.

The present inventors have demonstrated in Example 1 the construction of chimeric polypeptides with amino acid sequences encoding tags for purification, isolation, detection, imaging etc.

Accordingly, in one embodiment the chimeric polypeptide further comprises an amino acid sequence encoding a purification tag.

In one embodiment the purification tag is an AviTag or a Hexa-His tag.

In one embodiment, the present invention provides a chimeric polypeptide comprising the amino acid sequence

(SEQ ID NO: 14)
MFLIIFILPTTLAVIGDFNCTNSFINDYNKTIPRISEDVVDVSLGLGTYY
VLNRVYLNTTLLFTGYFPKSGANFRDLALKGSIYLSTLWYKPPFLSDFNN
GIFSKVKNTKLYVNNTLYSEFSTIVIGSVFVNTSYTIVVQPHNGILEITA
CQYTMCEYPHTVCKSKGSIRNESWHIDSSEPLCLFKKNFTYNVSADWLYF
HFYQERGVFYAYYADVGMPTTFLFSLYLGTILSHYYVMPLTCNAISSNTD
NETLEYWVTPLSRRQYLLNFDEHGVITNAVDCSSSFLSEIQCKTQSFAPN
TGVYDLSGFTVQPTESIVRFPNITNLCPFGEVFNATRFASVYAWNRKRIS
NCVADYSVLYNSASFSTFKCYGVSPTKLNDLCFTNVYADSFVIRGDEVRQ
IAPGQTGKIADYNYKLPDDFTGCVIAWNSNNLDSKVGGNYNYLYRLFRKS
NLKPFERDISTEIYQAGSTPCNGVEGFNCYFPLQSYGFQPTNGVGYQPYR
VVVLSFELLHAPATVCGPKKSTNLVKNKCVNYDLYGITGQGIFKEVSAAY
YNNWQNLLYDSNGNIIGFKDFLINKTYTILPCYSGRVSAAFYQNSSSPAL
LYRNLKCSYVLNNISFISQPFYFDSYLGCVLNAVNLTSYSVSSCDLRMGS
GFCIDYALPSSRASVGGISSPYRFVTFEPFNVSFVNDSVETVGGLFEIQI
PTNFTIAGHEEFIQTSSPKVTIDCSAFVCSNYAACHDLLSEYGTFCDNIN
SILNEVNDLLDITQLQVANALMQGVTLSSNLNTNLHSDVDNIDFKSLLGC
LGSQCGSSSRSPLEDLLFNKVKLSDVGFVEAYNNCTGGSEIRDLLCVQSF
NGIKVLPPILSETQISGYTTAATVAAMFPPWSAAAGVPFPLNVQYRINGL
GVTMDVLNKNQKLIANAFNKALLSIQNGFTATPSALAKIQSVVNANAQAL
NSLLQQLFNKFGAISSSLQEILSRLDPPEAQVQIDRLINGRLTALNAYVS
QQLSDITLIKAGASRAIEKVNECVKSQSPRINFCGNGNHILSLVQNAPYG
LLFIHFSYKPTSFKTVLVSPGLCLSGDRGIAPKQGYFIKQNDSWMFTGSS
YYYPEPISDKNVVFMNSCSVNFTKAPFIYLNNSIPNLSDFEAELSLWFKN
HTSIAPNLTFNSHINATFLDLYYEMNVIQESIKSLNSSFINLKEIGTYEM
GSGYIPEAPRDGQAYVRKDGEWVLLSTFLGSGLNDIFEAQKIEWHEGHHH
HHH
or
a variant thereof.

In another embodiment, the amino acid sequence encoding the chimeric polypeptide has at least 80% identity to the amino acid sequence of SEQ ID NO: 14.

In another embodiment, the amino acid sequence encoding the chimeric polypeptide has at least 90% identity to the amino acid sequence of SEQ ID NO: 14

In another embodiment, the present invention provides a chimeric polypeptide comprising the amino acid sequence

(SEQ ID NO: 15)
MFLIIFILPTTLAVIGDFNCTNSFINDYNKTIPRISEDVVDVSLGLGTYY
VLNRVYLNTTLLFTGYFPKSGANFRDLALKGSIYLSTLWYKPPFLSDFNN
GIFSKVKNTKLYVNNTLYSEFSTIVIGSVFVNTSYTIVVQPHNGILEITA
CQYTMCEYPHTVCKSKGSIRNESWHIDSSEPLCLFKKNFTYNVSADWLYF
HFYQERGVFYAYYADVGMPTTFLFSLYLGTILSHYYVMPLTCNAISSNTD
NETLEYWVTPLSRRQYLLNFDEHGVITNAVDCSSSFLSEIQCKTQSFAPN
TGVYDLSGFTVQPTESIVRFPNITNLCPFGEVFNATRFASVYAWNRKRIS
NCVADYSVLYNSASFSTFKCYGVSPTKLNDLCFTNVYADSFVIRGDEVRQ
IAPGQTGKIADYNYKLPDDFTGCVIAWNSNNLDSKVGGNYNYLYRLFRKS
NLKPFERDISTEIYQAGSTPCNGVEGFNCYFPLQSYGFQPTNGVGYQPYR
VVVLSFELLHAPATVCGPKKSTNLVKNKCVNYDLYGITGQGIFKEVSAAY
YNNWQNLLYDSNGNIIGFKDFLINKTYTILPCYSGRVSAAFYQNSSSPAL
LYRNLKCSYVLNNISFISQPFYFDSYLGCVLNAVNLTSYSVSSCDLRMGS
GFCIDYALPSSRASVGGISSPYRFVTFEPFNVSFVNDSVETVGGLFEIQI
PTNFTIAGHEEFIQTSSPKVTIDCSAFVCSNYAACHDLLSEYGTFCDNIN
SILNEVNDLLDITQLQVANALMQGVTLSSNLNTNLHSDVDNIDFKSLLGC
LGSQCGSSSRSPLEDLLFNKVKLSDVGFVEAYNNCTGGSEIRDLLCVQSF
NGIKVLPPILSETQISGYTTAATVAAMFPPWSAAAGVPFPLNVQYRINGL
GVTMDVLNKNQKLIANAFNKALLSIQNGFTATPSALAKIQSVVNANAQAL
NSLLQQLFNKFGAISSSLQEILSRLDPPEAQVQIDRLINGRLTALNAYVS
QQLSDITLIKAGASRAIEKVNECVKSQSPRINFCGNGNHILSLVQNAPYG
LLFIHFSYKPTSFKTVLVSPGLCLSGDRGIAPKQGYFIKQNDSWMFTGSS
YYYPEPISDKNVVFMNSCSVNFTKAPFIYLNNSIPNLSDFEAELSLWFKN
HTSIAPNLTFNSHINATFLDLYYEMNVIQESIKSLNSSFINLKEIGTYEM
GSGYIPEAPRDGQAYVRKDGEWVLLSTFL
or
a variant thereof.

In another embodiment, the amino acid sequence encoding the chimeric polypeptide has at least 80% identity to the amino acid sequence of SEQ ID NO: 15.

In another embodiment, the amino acid sequence encoding the chimeric polypeptide has at least 90% identity to the amino acid sequence of SEQ ID NO: 15.

In one embodiment, the present invention provides a chimeric polypeptide comprising the amino acid sequence

(SEQ ID NO: 16)
MFLIIFILPTTLAVIGDFNCTNSFINDYNKTIPRISEDVVDVSLGLGTYY
VLNRVYLNTTLLFTGYFPKSGANFRDLALKGSIYLSTLWYKPPFLSDFNN
GIFSKVKNTKLYVNNTLYSEFSTIVIGSVFVNTSYTIVVQPHNGILEITA
CQYTMCEYPHTVCKSKGSIRNESWHIDSSEPLCLFKKNFTYNVSADWLYF
HFYQERGVFYAYYADVGMPTTFLFSLYLGTILSHYYVMPLTCNAISSNTD
NETLEYWVTPLSRRQYLLNFDEHGVITNAVDCSSSFLSEIQCKTQSFAPN
TGVYDLSGFTVQPTESIVRFPNITNLCPFGEVFNATRFASVYAWNRKRIS
NCVADYSVLYNSASFSTFKCYGVSPTKLNDLCFTNVYADSFVIRGDEVRQ
IAPGQTGKIADYNYKLPDDFTGCVIAWNSNNLDSKVGGNYNYLYRLFRKS
NLKPFERDISTEIYQAGSTPCNGVEGFNCYFPLQSYGFQPTNGVGYQPYR
VVVLSFELLHAPATVCGPKKSTNLVKNKCVNYDLYGITGQGIFKEVSAAY
YNNWQNLLYDSNGNIIGFKDFLINKTYTILPCYSGRVSAAFYQNSSSPAL
LYRNLKCSYVLNNISFISQPFYFDSYLGCVLNAVNLTSYSVSSCDLRMGS
GFCIDYALPSSRASVGGISSPYRFVTFEPFNVSFVNDSVETVGGLFEIQI
PTNFTIAGHEEFIQTSSPKVTIDCSAFVCSNYAACHDLLSEYGTFCDNIN
SILNEVNDLLDITQLQVANALMQGVTLSSNLNTNLHSDVDNIDFKSLLGC
LGSQCGSSSRSPLEDLLFNKVKLSDVGFVEAYNNCTGGSEIRDLLCVQSF
NGIKVLPPILSETQISGYTTAATVAAMFPPWSAAAGVPFPLNVQYRINGL
GVTMDVLNKNQKLIANAFNKALLSIQNGFTATPSALAKIQSVVNANAQAL
NSLLQQLFNKFGAISSSLQEILSRLDPPEAQVQIDRLINGRLTALNAYVS
QQLSDITLIKAGASRAIEKVNECVKSQSPRINFCGNGNHILSLVQNAPYG
LLFIHFSYKPTSFKTVLVSPGLCLSGDRGIAPKQGYFIKQNDSWMFTGSS
YYYPEPISDKNVVFMNSCSVNFTKAPFIYLNNSIPNLSDFEAELSLWFKN
HTSIAPNLTFNSHINATFLDLYYEMNVIQESIKSLNSSFINLKEIGTYEM
GSGYIPEAPRDGQAYVRKDGEWVLLSTFL
or
a variant thereof.

In another embodiment, the amino acid sequence encoding the chimeric polypeptide has at least 80% identity to the amino acid sequence of SEQ ID NO: 16.

In another embodiment, the amino acid sequence encoding the chimeric polypeptide has at least 90% identity to the amino acid sequence of SEQ ID NO: 16.

In one embodiment, the present invention provides a chimeric polypeptide the amino acid sequence comprising

(SEQ ID NO: 17)
MFLIIFILPTTLAVIGDFNCTNSFINDYNKTIPRISEDVVDVSLGLGTYY
VLNRVYLNTTLLFTGYFPKSGANFRDLALKGSIYLSTLWYKPPFLSDFNN
GIFSKVKNTKLYVNNTLYSEFSTIVIGSVFVNTSYTIVVQPHNGILEITA
CQYTMCEYPHTVCKSKGSIRNESWHIDSSEPLCLFKKNFTYNVSADWLYF
HFYQERGVFYAYYADVGMPTTFLFSLYLGTILSHYYVMPLTCNAISSNTD
NETLEYWVTPLSRRQYLLNFDEHGVITNAVDCSSSFLSEIQCKTQSFAPN
TGVYDLSGFTVQPTESIVRFPNITNLCPFGEVFNATRFASVYAWNRKRIS
NCVADYSVLYNSASFSTFKCYGVSPTKLNDLCFTNVYADSFVIRGDEVRQ
IAPGQTGKIADYNYKLPDDFTGCVIAWNSNNLDSKVGGNYNYLYRLFRKS
NLKPFERDISTEIYQAGSTPCNGVEGFNCYFPLQSYGFQPTNGVGYQPYR
VVVLSFELLHAPATVCGPKKSTNLVKNKCVNYDLYGITGQGIFKEVSAAY
YNNWQNLLYDSNGNIIGFKDFLINKTYTILPCYSGRVSAAFYQNSSSPAL
LYRNLKCSYVLNNISFISQPFYFDSYLGCVLNAVNLTSYSVSSCDLRMGS
GFCIDYALPSSRASVGGISSPYRFVTFEPFNVSFVNDSVETVGGLFEIQI
PTNFTIAGHEEFIQTSSPKVTIDCSAFVCSNYAACHDLLSEYGTFCDNIN
SILNEVNDLLDITQLQVANALMQGVTLSSNLNTNLHSDVDNIDFKSLLGC
LGSQCGSSSRSPLEDLLFNKVKLSDVGFVEAYNNCTGGSEIRDLLCVQSF
NGIKVLPPILSETQISGYTTAATVAAMFPPWSAAAGVPFPLNVQYRINGL
GVTMDVLNKNQKLIANAFNKALLSIQNGFTATPSALAKIQSVVNANAQAL
NSLLQQLFNKFGAISSSLQEILSRLDPPEAQVQIDRLINGRLTALNAYVS
QQLSDITLIKAGASRAIEKVNECVKSQSPRINFCGNGNHILSLVQNAPYG
LLFIHFSYKPTSFKTVLVSPGLCLSGDRGIAPKQGYFIKQNDSWMFTGSS
YYYPEPISDKNVVFMNSCSVNFTKAPFIYLNNSIPNLSDFEAELSLWFKN
HTSIAPNLTFNSHINATFLDLYYEMNVIQESIKSLNSSFINLKEIGTYEM
GSGYIPEAPRDGQAYVRKDGEWVLLSTFLGSHHHHHH
or
a variant thereof.

In another embodiment, the amino acid sequence encoding the chimeric polypeptide has at least 80% identity to the amino acid sequence of SEQ ID NO: 17.

In another embodiment, the amino acid sequence encoding the chimeric polypeptide has at least 90% identity to the amino acid sequence of SEQ ID NO: 17.

In one embodiment, the present invention provides a chimeric polypeptide comprising

(SEQ ID NO: 24)
MFLIIFILPTTLAVIGDFNCTNSFINDYNKTIPRISEDVVDVSLGLGTYY
VLNRVYLNTTLLFTGYFPKSGANFRDLALKGSIYLSTLWYKPPFLSDFNN
GIFSKVKNTKLYVNNTLYSEFSTIVIGSVFVNTSYTIVVQPHNGILEITA
CQYTMCEYPHTVCKSKGSIRNESWHIDSSEPLCLFKKNFTYNVSADWLYF
HFYQERGVFYAYYADVGMPTTFLFSLYLGTILSHYYVMPLTCNAISSNTD
NETLEYWVTPLSRRQYLLNFDEHGVITNAVDCSSSFLSEIQCKTQSFAPN
TGVYDLSGFTVQPTESIVRFPNITNLCPFDEVFNATRFASVYAWNRKRIS
NCVADYSVLYNLAPFFTFKCYGVSPTKLNDLCFTNVYADSFVIRGDEVRQ
IAPGQTGNIADYNYKLPDDFTGCVIAWNSNKLDSKVSGNYNYLYRLFRKS
NLKPFERDISTEIYQAGNKPCNGVAGFNCYFPLRSYSFRPTYGVGHQPYR
VVVLSFELLHAPATVCGPKKSTNLVKNKCVNYDLYGITGQGIFKEVSAAY
YNNWQNLLYDSNGNIIGFKDFLINKTYTILPCYSGRVSAAFYQNSSSPAL
LYRNLKCSYVLNNISFISQPFYFDSYLGCVLNAVNLTSYSVSSCDLRMGS
GFCIDYALPSSRASVGGISSPYRFVTFEPFNVSFVNDSVETVGGLFEIQI
PTNFTIAGHEEFIQTSSPKVTIDCSAFVCSNYAACHDLLSEYGTFCDNIN
SILNEVNDLLDITQLQVANALMQGVTLSSNLNTNLHSDVDNIDFKSLLGC
LGSQCGSSSRSPLEDLLFNKVKLSDVGFVEAYNNCTGGSEIRDLLCVQSF
NGIKVLPPILSETQISGYTTAATVAAMFPPWSAAAGVPFPLNVQYRINGL
GVTMDVLNKNQKLIANAFNKALLSIQNGFTATPSALAKIQSVVNANAQAL
NSLLQQLFNKFGAISSSLQEILSRLDPPEAQVQIDRLINGRLTALNAYVS
QQLSDITLIKAGASRAIEKVNECVKSQSPRINFCGNGNHILSLVQNAPYG
LLFIHFSYKPTSFKTVLVSPGLCLSGDRGIAPKQGYFIKQNDSWMFTGSS
YYYPEPISDKNVVFMNSCSVNFTKAPFIYLNNSIPNLSDFEAELSLWFKN
HTSIAPNLTFNSHINATFLDLYYEMNVIQESIKSLNSSFINLKEIGTYEM
GSGYIPEAPRDGQAYVRKDGEWVLLSTFL
or
a variant thereof.

In another embodiment, the amino acid sequence encoding the chimeric polypeptide has at least 80% identity to the amino acid sequence of SEQ ID NO: 24.

In another embodiment, the amino acid sequence encoding the chimeric polypeptide has at least 90% identity to the amino acid sequence of SEQ ID NO: 24.

In one embodiment, the present invention provides a chimeric polypeptide comprising the amino acid sequence

(SEQ ID NO: 25)
MFLIIFILPTTLAVIGDFNCTNSFINDYNKTIPRISEDVVDVSLGLGTY
YVLNRVYLNTTLLFTGYFPKSGANFRDLALKGSIYLSTLWYKPPFLSDF
NNGIFSKVKNTKLYVNNTLYSEFSTIVIGSVFVNTSYTIVVQPHNGILE
ITACQYTMCEYPHTVCKSKGSIRNESWHIDSSEPLCLFKKNFTYNVSAD
WLYFHFYQERGVFYAYYADVGMPTTFLFSLYLGTILSHYYVMPLTCNAI
SSNTDNETLEYWVTPLSRRQYLLNFDEHGVITNAVDCSSSFLSEIQCKT
QSFAPNTGVYDLSGFTVQPTESIVRFPNITNLCPFDEVFNATRFASVYA
WNRKRISNCVADYSVLYNLAPFFTFKCYGVSPTKLNDLCFTNVYADSFV
IRGDEVRQIAPGQTGNIADYNYKLPDDFTGCVIAWNSNKLDSKVSGNYN
YLYRLFRKSNLKPFERDISTEIYQAGNKPCNGVAGFNCYFPLRSYSFRP
TYGVGHQPYRVVVLSFELLHAPATVCGPKKSTNLVKNKCVNYDLYGITG
QGIFKEVSAAYYNNWQNLLYDSNGNIIGFKDFLTNKTYTIPCYSGRVSA
AFYQNSSSPALLYRNLKCSYVLNNISFISQPFYFDSYLGCVLNAVNLTS
YSVSSCDLRMGSGFCIDYALPSSRASVGGISSPYRFVTFEPFNVSFVND
SVETVGGLFEIQIPTNFTIAGHEEFIQTSSPKVTIDCSAFVCSNYAACH
DLLSEYGTFCDNINSILNEVNDLLDITQLQVANALMQGVTLSSNLNTNL
HSDVDNIDFKSLLGCLGSQCGSSSRSPLEDLLFNKVKLSDVGFVEAYNN
CTGGSEIRDLLCVQSFNGIKVLPPILSETQISGYTTAATVAAMFPPWSA
AAGVPFPLNVQYRINGLGVTMDVLNKNQKLIANAFNKALLSIQNGFTAT
PSALAKIQSVVNANAQALNSLLQQLFNKFGAISSSLQEILSRLDPPEAQ
VQIDRLINGRLTALNAYVSQQLSDITLIKAGASRAIEKVNECVKSQSPR
INFCGNGNHILSLVQNAPYGLLFIHFSYKPTSFKTVLVSPGLCLSGDRG
IAPKQGYFIKQNDSWMFTGSSYYYPEPISDKNVVFMNSCSVNFTKAPFI
YLNNSIPNLSDFEAELSLWFKNHTSIAPNLTFNSHINATFLDLYYEMNV
IQESIKSLNSSFINLKEIGTYEMGSGYIPEAPRDGQAYVRKDGEWVLLS
TFLGSGLNDIFEAQKIEWHEGHHHHHH
or
a variant thereof.

In another embodiment, the amino acid sequence encoding the chimeric polypeptide has at least 80% identity to the amino acid sequence of SEQ ID NO: 25.

In another embodiment, the amino acid sequence encoding the chimeric polypeptide has at least 90% identity to the amino acid sequence of SEQ ID NO: 25.

In one embodiment, the present invention provides a chimeric polypeptide comprising

(SEQ ID NO: 26)
MFLIIFILPTTLAVIGDFNCTNSFINDYNKTIPRISEDVVDVSLGLGTYY
VLNRVYLNTTLLFTGYFPKSGANFRDLALKGSIYLSTLWYKPPFLSDFNN
GIFSKVKNTKLYVNNTLYSEFSTIVIGSVFVNTSYTIVVQPHNGILEITA
CQYTMCEYPHTVCKSKGSIRNESWHIDSSEPLCLFKKNFTYNVSADWLYF
HFYQERGVFYAYYADVGMPTTFLFSLYLGTILSHYYVMPLTCNAISSNTD
NETLEYWVTPLSRRQYLLNFDEHGVITNAVDCSSSFLSEIQCKTQSFAPN
TGVYDLSGFTVQPTESIVRFPNITNLCPFDEVFNATRFASVYAWNRKRIS
NCVADYSVLYNLAPFFTFKCYGVSPTKLNDLCFTNVYADSFVIRGDEVRQ
IAPGQTGNIADYNYKLPDDFTGCVIAWNSNKLDSKVSGNYNYLYRLFRKS
NLKPFERDISTEIYQAGNKPCNGVAGFNCYFPLRSYSFRPTYGVGHQPYR
VVVLSFELLHAPATVCGPKKSTNLVKNKCVNYDLYGITGQGIFKEVSAAY
YNNWQNLLYDSNGNIIGFKDFLTNKTYTILPCYSGRVSAAFYQNSSSPAL
LYRNLKCSYVLNNISFISQPFYFDSYLGCVLNAVNLTSYSVSSCDLRMGS
GFCIDYALPSSRASVGGISSPYRFVTFEPFNVSFVNDSVETVGGLFEIQI
PTNFTIAGHEEFIQTSSPKVTIDCSAFVCSNYAACHDLLSEYGTFCDNIN
SILNEVNDLLDITQLQVANALMQGVTLSSNLNTNLHSDVDNIDFKSLLGC
LGSQCGSSSRSPLEDLLFNKVKLSDVGFVEAYNNCTGGSEIRDLLCVQSF
NGIKVLPPILSETQISGYTTAATVAAMFPPWSAAAGVPFPLNVQYRINGL
GVTMDVLNKNQKLIANAFNKALLSIQNGFTATPSALAKIQSVVNANAQAL
NSLLQQLFNKFGAISSSLQEILSRLDPPEAQVQIDRLINGRLTALNAYVS
QQLSDITLIKAGASRAIEKVNECVKSQSPRINFCGNGNHILSLVQNAPYG
LLFIHFSYKPTSFKTVLVSPGLCLSGDRGIAPKQGYFIKQNDSWMFTGSS
YYYPEPISDKNVVFMNSCSVNFTKAPFIYLNNSIPNLSDFEAELSLWFKN
HTSIAPNLTFNSHINATFLDLYYEMNVIQESIKSLNSSFINLKEIGTYEM
GSGYIPEAPRDGQAYVRKDGEWVLLSTFLGSGYIPEAPRDGQAYVRKDGE
WVLLSTFLGGGGSGGGGSGGGGSHHHHHH*
or
a variant thereof.

In another embodiment, the amino acid sequence encoding the chimeric polypeptide has at least 80% identity to the amino acid sequence of SEQ ID NO: 26.

In another embodiment, the amino acid sequence encoding the chimeric polypeptide has at least 90% identity to the amino acid sequence of SEQ ID NO: 26.

In one embodiment, the present invention provides a chimeric polypeptide comprising the amino acid sequence

(SEQ ID NO: 27)
MFLIIFILPTTLAVIGDFNCTNSFINDYNKTIPRISEDVVDVSLGLGTYYVLNRVYLNTTL
LFTGYFPKSGANFRDLALKGSIYLSTLWYKPPFLSDFNNGIFSKVKNTKLYVNNTLYS
EFSTIVIGSVFVNTSYTIVVQPHNGILEITACQYTMCEYPHTVCKSKGSIRNESWHIDS
SEPLCLFKKNFTYNVSADWLYFHFYQERGVFYAYYADVGMPTTFLFSLYLGTILSHY
YVMPLTCNAISSNTDNETLEYWVTPLSRRQYLLNFDEHGVITNAVDCSSSFLSEIQCK
TQSFAPNTGVYDLSGFTVQPTESIVRFPNITNLCPFDEVFNATRFASVYAWNRKRIS
NCVADYSVLYNFAPFFAFKCYGVSPTKLNDLCFTNVYADSFVIRGNEVSQIAPGQTG
NIADYNYKLPDDFTGCVIAWNSNKLDSKVGGNYNYLYRLFRKSNLKPFERDISTEIYQ
AGNKPCNGVAGFNCYFPLRSYGFRPTYGVGHQPYRVVVLSFELLHAPATVCGPKK
STNLVKNKCVNYDLYGITGQGIFKEVSAAYYNNWQNLLYDSNGNIIGFKDFLINKTYT
ILPCYSGRVSAAFYQNSSSPALLYRNLKCSYVLNNISFISQPFYFDSYLGCVLNAVNL
TSYSVSSCDLRMGSGFCIDYALPSSRASVGGISSPYRFVTFEPFNVSFVNDSVETVG
GLFEIQIPTNFTIAGHEEFIQTSSPKVTIDCSAFVCSNYAACHDLLSEYGTFCDNINSIL
NEVNDLLDITQLQVANALMQGVTLSSNLNTNLHSDVDNIDFKSLLGCLGSQCGSSSR
SPLEDLLFNKVKLSDVGFVEAYNNCTGGSEIRDLLCVQSFNGIKVLPPILSETQISGYT
TAATVAAMFPPWSAAAGVPFPLNVQYRINGLGVTMDVLNKNQKLIANAFNKALLSIQ
NGFTATPSALAKIQSVVNANAQALNSLLQQLFNKFGAISSSLQEILSRLDPPEAQVQI
DRLINGRLTALNAYVSQQLSDITLIKAGASRAIEKVNECVKSQSPRINFCGNGNHILSL
VQNAPYGLLFIHFSYKPTSFKTVLVSPGLCLSGDRGIAPKQGYFIKQNDSWMFTGSS
YYYPEPISDKNVVFMNSCSVNFTKAPFIYLNNSIPNLSDFEAELSLWFKNHTSIAPNLT
FNSHINATFLDLYYEMNVIQESIKSLNSSFINLKEIGTYEMGSGYIPEAPRDGQAYVRK
DGEWVLLSTFL
or
a variant thereof.

In another embodiment, the amino acid sequence encoding the chimeric polypeptide has at least 80% identity to the amino acid sequence of SEQ ID NO: 27.

In another embodiment, the amino acid sequence encoding the chimeric polypeptide has at least 90% identity to the amino acid sequence of SEQ ID NO: 27.

In one embodiment, the present invention provides a chimeric polypeptide comprising he amino acid sequence

((SEQ ID NO: 28)
MFLIIFILPTTLAVIGDFNCTNSFINDYNKTIPRISEDVVDVSLGLGTYYVLNRVYLNTTL
LFTGYFPKSGANFRDLALKGSIYLSTLWYKPPFLSDFNNGIFSKVKNTKLYVNNTLYS
EFSTIVIGSVFVNTSYTIVVQPHNGILEITACQYTMCEYPHTVCKSKGSIRNESWHIDS
SEPLCLFKKNFTYNVSADWLYFHFYQERGVFYAYYADVGMPTTFLFSLYLGTILSHY
YVMPLTCNAISSNTDNETLEYWVTPLSRRQYLLNFDEHGVITNAVDCSSSFLSEIQCK
TQSFAPNTGVYDLSGFTVQPTESIVRFPNITNLCPFDEVFNATRFASVYAWNRKRIS
NCVADYSVLYNFAPFFAFKCYGVSPTKLNDLCFTNVYADSFVIRGNEVSQIAPGQTG
NIADYNYKLPDDFTGCVIAWNSNKLDSKVGGNYNYLYRLFRKSNLKPFERDISTEIYQ
AGNKPCNGVAGFNCYFPLRSYGFRPTYGVGHQPYRVVVLSFELLHAPATVCGPKK
STNLVKNKCVNYDLYGITGQGIFKEVSAAYYNNWQNLLYDSNGNIIGFKDFLINKTYT
ILPCYSGRVSAAFYQNSSSPALLYRNLKCSYVLNNISFISQPFYFDSYLGCVLNAVNL
TSYSVSSCDLRMGSGFCIDYALPSSRASVGGISSPYRFVTFEPFNVSFVNDSVETVG
GLFEIQIPTNFTIAGHEEFIQTSSPKVTIDCSAFVCSNYAACHDLLSEYGTFCDNINSIL
NEVNDLLDITQLQVANALMQGVTLSSNLNTNLHSDVDNIDFKSLLGCLGSQCGSSSR
SPLEDLLFNKVKLSDVGFVEAYNNCTGGSEIRDLLCVQSFNGIKVLPPILSETQISGYT
TAATVAAMFPPWSAAAGVPFPLNVQYRINGLGVTMDVLNKNQKLIANAFNKALLSIQ
NGFTATPSALAKIQSVVNANAQALNSLLQQLFNKFGAISSSLQEILSRLDPPEAQVQI
DRLINGRLTALNAYVSQQLSDITLIKAGASRAIEKVNECVKSQSPRINFCGNGNHILSL
VQNAPYGLLFIHFSYKPTSFKTVLVSPGLCLSGDRGIAPKQGYFIKQNDSWMFTGSS
YYYPEPISDKNVVFMNSCSVNFTKAPFIYLNNSIPNLSDFEAELSLWFKNHTSIAPNLT
FNSHINATFLDLYYEMNVIQESIKSLNSSFINLKEIGTYEMGSGYIPEAPRDGQAYVRK
DGEWVLLSTFLGSGLNDIFEAQKIEWHEGHHHHHH
or
a variant thereof.

In another embodiment, the amino acid sequence encoding the chimeric polypeptide has at least 80% identity to the amino acid sequence of SEQ ID NO: 28.

In another embodiment, the amino acid sequence encoding the chimeric polypeptide has at least 90% identity to the amino acid sequence of SEQ ID NO: 28.

In one embodiment, the present invention provides a chimeric polypeptide amino acid comprising the sequence

(SEQ ID NO: 29)
MFLIIFILPTTLAVIGDFNCTNSFINDYNKTIPRISEDVVDVSLGLGTYYVLNRVYLNTTL
LFTGYFPKSGANFRDLALKGSIYLSTLWYKPPFLSDFNNGIFSKVKNTKLYVNNTLYS
EFSTIVIGSVFVNTSYTIVVQPHNGILEITACQYTMCEYPHTVCKSKGSIRNESWHIDS
SEPLCLFKKNFTYNVSADWLYFHFYQERGVFYAYYADVGMPTTFLFSLYLGTILSHY
YVMPLTCNAISSNTDNETLEYWVTPLSRRQYLLNFDEHGVITNAVDCSSSFLSEIQCK
TQSFAPNTGVYDLSGFTVQPTESIVRFPNITNLCPFDEVFNATRFASVYAWNRKRIS
NCVADYSVLYNFAPFFAFKCYGVSPTKLNDLCFTNVYADSFVIRGNEVSQIAPGQTG
NIADYNYKLPDDFTGCVIAWNSNKLDSKVGGNYNYLYRLFRKSNLKPFERDISTEIYQ
AGNKPCNGVAGFNCYFPLRSYGFRPTYGVGHQPYRVVVLSFELLHAPATVCGPKK
STNLVKNKCVNYDLYGITGQGIFKEVSAAYYNNWQNLLYDSNGNIIGFKDFLINKTYT
ILPCYSGRVSAAFYQNSSSPALLYRNLKCSYVLNNISFISQPFYFDSYLGCVLNAVNL
TSYSVSSCDLRMGSGFCIDYALPSSRASVGGISSPYRFVTFEPFNVSFVNDSVETVG
GLFEIQIPTNFTIAGHEEFIQTSSPKVTIDCSAFVCSNYAACHDLLSEYGTFCDNINSIL
NEVNDLLDITQLQVANALMQGVTLSSNLNTNLHSDVDNIDFKSLLGCLGSQCGSSSR
SPLEDLLFNKVKLSDVGFVEAYNNCTGGSEIRDLLCVQSFNGIKVLPPILSETQISGYT
TAATVAAMFPPWSAAAGVPFPLNVQYRINGLGVTMDVLNKNQKLIANAFNKALLSIQ
NGFTATPSALAKIQSVVNANAQALNSLLQQLFNKFGAISSSLQEILSRLDPPEAQVQI
DRLINGRLTALNAYVSQQLSDITLIKAGASRAIEKVNECVKSQSPRINFCGNGNHILSL
VQNAPYGLLFIHFSYKPTSFKTVLVSPGLCLSGDRGIAPKQGYFIKQNDSWMFTGSS
YYYPEPISDKNVVFMNSCSVNFTKAPFIYLNNSIPNLSDFEAELSLWFKNHTSIAPNLT
FNSHINATFLDLYYEMNVIQESIKSLNSSFINLKEIGTYEMGSGYIPEAPRDGQAYVRK
DGEWVLLSTFLGSGYIPEAPRDGQAYVRKDGEWVLLSTFLGGGGSGGGGSGGGG
SHHHHHH
or
a variant thereof.

In another embodiment, the amino acid sequence encoding the chimeric polypeptide has at least 80% identity to the amino acid sequence of SEQ ID NO: 29.

In another embodiment, the amino acid sequence encoding the chimeric polypeptide has at least 90% identity to the amino acid sequence of SEQ ID NO: 29.

The present invention also provides for nucleic acids encoding a chimeric polypeptide as described herein.

The term “nucleic acid” includes any compound and/or substance that comprises a polymer of nucleotides (nucleotide monomer). These polymers are referred to as polynucleotides. Thus, the terms “nucleic acid” and “polynucleotide” are used interchangeably.

Nucleic acids may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or chimeras or combinations thereof.

In some embodiments, polynucleotides of the present disclosure function as messenger RNA (mRNA). “Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. The skilled artisan will appreciate that, except where otherwise noted, polynucleotide sequences set forth in the instant application will recite “T”s in a representative DNA sequence but where the sequence represents RNA (e.g., mRNA), the “T”s would be substituted for “U”s. Thus, any of the RNA polynucleotides encoded by a DNA identified by a particular sequence identification number may also comprise the corresponding RNA (e.g., mRNA) sequence encoded by the DNA, where each “T” of the DNA sequence is substituted with “U.”

The basic components of an mRNA molecule typically include at least one coding region, a 5′ untranslated region (UTR), a 3′ UTR, a 5′ cap and a poly-A tail. Polynucleotides of the present disclosure may function as mRNA but can be distinguished from wild-type mRNA in their functional and/or structural design features, which serve to overcome existing problems of effective polypeptide expression using nucleic-acid based therapeutics.

In some embodiments, an RNA polynucleotide of an RNA (e.g., mRNA) vaccine encodes more than one antigenic polypeptides, including the chimeric polypeptides described herein.

Polynucleotides of the present disclosure, in some embodiments, are codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art-non-limiting examples include services from GeneArt (Life Technologies), DNA2.0 (Menlo Park Calif.) and/or proprietary methods. In some embodiments, the open reading frame (ORF) sequence is optimized using optimization algorithms.

In some embodiments, a codon optimized sequence shares less than 95% sequence identity, less than 90% sequence identity, less than 85% sequence identity, less than 80% sequence identity, or less than 75% sequence identity to a naturally-occurring or wild-type sequence (e.g., a naturally-occurring or wild-type mRNA sequence encoding a polypeptide or protein of interest (e.g., an antigenic protein or antigenic polypeptide, including the chimeric polypeptides described herein).

In some embodiments, a codon-optimized sequence shares between 65% and 85% (e.g., between about 67% and about 85%, or between about 67% and about 80%) sequence identity to a naturally-occurring sequence or a wild-type sequence (e.g., a naturally-occurring or wild-type mRNA sequence encoding a polypeptide or protein of interest (e.g., an antigenic protein or polypeptide)). In some embodiments, a codon-optimized sequence shares between 65% and 75%, or about 80% sequence identity to a naturally-occurring sequence or wild-type sequence (e.g., a naturally-occurring or wild-type mRNA sequence encoding a polypeptide or protein of interest (e.g., an antigenic protein or polypeptide)).

In some embodiments a codon-optimized RNA (e.g., mRNA) may, for instance, be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. Due to the degeneracy of the genetic code, the modifications work by substituting existing codons for those that promote greater RNA stability without changing the resulting amino acid. The approach is limited to coding regions of the RNA.

In one embodiment, the nucleic acid is RNA (e.g. mRNA) or DNA.

In one embodiment, the mRNA further comprised a 5′ untranslated region (UTR) and a 3′ UTR.

A “5′ untranslated region” (5′UTR) as used herein refers to a region of an mRNA that is directly upstream (i.e., 5′) from the start codon (i.e., the first codon of an mRNA transcript translated by a ribosome) that does not encode a polypeptide.

A “3′ untranslated region” (3′UTR) as used herein refers to a region of an mRNA that is directly downstream (i.e., 3′) from the stop codon (i.e., the codon of an mRNA transcript that signals a termination of translation) that does not encode a polypeptide.

In another embodiment, the mRNA further comprises a poly (A) tail.

A “polyA tail” as used herein refers to a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly (A) tail functions to protect mRNA from enzymatic degradation, e.g., in the cytoplasm, and aids in transcription termination, export of the mRNA from the nucleus and translation.

In one embodiment, the mRNA comprises a chemical modification. Suitable modifications to mRNA are known in the art and include pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

In one embodiment the nucleic acid encodes a trimerization domain to allow a chimeric polypeptide encoded by the nucleic acid to form a trimer. In a preferred embodiment, the nucleic acid encoding a chimeric polypeptide as described herein further encodes the transmembrane domain and cytoplasmic tail of the first betacoronavirus spike protein to allow trimerisation of the chimeric polypeptide encoded by the nucleic acid.

In one embodiment, the present invention provides a vector comprising a nucleic acid as described herein.

As used herein the term “vector” includes a nucleic acid molecule into which a second nucleic acid molecule can be inserted for introduction into a host where it will be replicated, and in some cases expressed. In other words, a vector is capable of transporting a nucleic acid molecule to which it has been linked. Cloning as well as expression vectors are contemplated by the term “vector”, as used herein. Vectors include, but are not limited to, plasmids, cosmids, bacterial artificial chromosomes (BAC) and yeast artificial chromosomes (YAC) and vectors derived from bacteriophages or plant or animal (including human) viruses. Vectors comprise an origin of replication recognised by the proposed host and in case of expression vectors, promoter and other regulatory regions recognised by the host. A vector containing a second nucleic acid molecule is introduced into a cell by transformation, transfection, or by making use of viral entry mechanisms. Certain vectors are capable of autonomous replication in a host into which they are introduced (e.g., vectors having a bacterial origin of replication can replicate in bacteria). Other vectors can be integrated into the genome of a host upon introduction into the host, and thereby are replicated along with the host genome.

In another embodiment, the present invention provides a host cell comprising a nucleic acid as described herein.

Importantly, the present inventors have demonstrated in Example 3 that a Chimeric Trimeric RBD vaccine as described herein induces neutralising and anti-RBD antibodies in vivo, and at higher titres than convalescent humans following COVID-19 infection.

Example 4 demonstrates that a Chimeric Trimeric RBD vaccine as described herein induces robust CD4 T follicular helper cell responses in vivo. This data confirms that his confirms that the N terminal domain of the first betacoronavirus spike protein and C terminal region of the first coronavirus spike protein provide T cell epitopes that are lacking in the RBD of the second betacoronavirus spike protein, which can support an antibody response to the vaccine.

Example 5 demonstrates that a Chimeric Trimeric RBD vaccine as described herein induces near complete suppression of viral replication in the lungs of mice, including durable sterilising protection after a single CTR-WT injection.

Example 6 demonstrates that a Chimeric Trimeric RBD vaccine induces neutralising and anti-RBD antibodies in non-human primates, and at higher titres than convalescent humans following COVID-19 infection.

Accordingly, in one embodiment, the present invention provides a vaccine composition comprising a chimeric polypeptide as described herein, or a trimer thereof, or a nucleic acid as described herein, and a pharmaceutically acceptable carrier.

The term “vaccine” includes a substance, (e.g. a protein such as a CTR protein, or an RNA encoding a polypeptide described herein), which is capable of inducing an immune response in a subject. The term also refers to proteins, or RNAs that encode proteins, that are immunologically active in the sense that once administered to a subject (either directly or by administering to the subject a nucleotide sequence or vector that encodes the protein) is able to evoke an immune response of the humoral and/or cellular type directed against that protein.

The term “pharmaceutically acceptable carrier” includes any inert substance that is combined with a composition comprising a chimeric polypeptide as described herein, or a trimer thereof, or a nucleic acid as described herein. The “pharmaceutically acceptable carrier” is an excipient that is non-toxic to recipients at the dosages and concentrations employed and is compatible with other ingredients of the formulation. For example, sterile water or physiological saline may be used. Other substances, such as pH buffering solutions, viscosity reducing agents, or stabilizers may also be included.

A wide variety of pharmaceutically acceptable excipients and carriers are known in the art. Such pharmaceutical carriers and excipients as well as suitable pharmaceutical formulations have been amply described in a variety of publications (see for example “Pharmaceutical Formulation Development of Peptides and Proteins”, Frokjaeret al., Taylor & Francis (2000) or “Handbook of Pharmaceutical Excipients”, 3rd edition, Kibbe et al., Pharmaceutical Press (2000) A. Gennaro (2000) “Remington: The Science and Practice of Pharmacy”, 20th edition, Lippincott, Williams, & WIkins; Pharmaceutical Dosage Forms and Drug Delivery Systems (1999) H. C. Ansel et al., eds 7th ed., Lippincott, Wiliams, & WIkins; and Handbook of Pharmaceutical Excipients (2000) A. H. Kibbe et al., eds., 3rd ed. Amer. Pharmaceutical Assoc). In particular, the pharmaceutical composition comprising the antibody of the invention may be formulated in lyophilized or stable soluble form. The chimeric polypeptide as described herein, or a trimer thereof, may be lyophilized by a variety of procedures known in the art. Lyophilized formulations are reconstituted prior to use by the addition of one or more pharmaceutically acceptable diluents such as sterile water for injection or sterile physiological saline solution.

Pharmaceutical compositions comprising an antibody or fragments thereof described herein can be administered in dosages and by techniques well known in the art. The amount and timing of the administration will be determined by the treating physician to achieve the desired purposes and should ensure a delivery of a safe and therapeutically effective dose to the blood of the subject to be treated.

In one embodiment, the composition is administered in an amount to induce an effective antibody and/or T cell response in the subject.

Without wishing to be bound by theory, the present inventors propose that the extent of viral suppression in vivo correlated with in vitro measurements of neutralising activity but not binding affinity, suggesting functional potency is a key defining metric for protective efficacy. Accordingly, in a preferred embodiment, the effective antibody response is a neutralising antibody response.

As used herein the term “neutralises SARS-COV-2” or “a neutralising antibody response” refers to reducing the infectivity of SARS-COV-2, for example, by inhibiting the attachment of SARS-Co-2 to receptors on host cells. In a specific embodiment, the binding molecules of the invention prevent SARS-Co-2 from infecting host cells by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to infection of host cells by SARS-COV in the absence of said binding molecules. Neutralisation can for instance be measured as described herein.

In one embodiment, neutralisation is determined by a method as described herein, such as neutralisation assays with suitable cells, such as Vero cells. Example 1 sets out a microneutralisation assay with ELISA-based read out.

In another embodiment, the composition can additionally include one or more other therapeutic ingredients (for example, antiviral drugs).

The present inventors describe herein in the Examples pharmaceutical compositions comprising adjuvants selected from the group consisting of Addavax, MF59, and MPLA liposomal adjuvant. Accordingly, in another embodiment, the composition can additionally include one or more adjuvants. Examples of suitable adjuvants include, e.g., aluminum hydroxide, lecithin, Freund's adjuvant, MPL™ and IL-12. In some embodiments, the vaccine compositions or nanoparticle immunogens disclosed herein (e.g., SARS-COV-2 vaccine composition) can be formulated as a controlled-release or time-release formulation. This can be achieved in a composition that contains a slow release polymer or via a microencapsulated delivery system or bioadhesive gel. The various pharmaceutical compositions can be prepared in accordance with standard procedures well known in the art. See, e.g., Remington's Pharmaceutical Sciences, 19 th Ed., Mack Publishing Company, Easton, Pa., 1995; Sustained and Controlled Release Drug Delivery Systems, J. R. Robinson, ed., Marcel Dekker, Inc., New York, 1978); U.S. Pat. Nos. 4,652,441 and 4,917,893; 4,677,191 and 4,728,721; and 4,675,189.

Thus, some of the pharmaceutical compositions of the invention are vaccine compositions. For vaccine compositions, appropriate adjuvants can be additionally included. Examples of suitable adjuvants include, e.g., aluminum hydroxide, lecithin, Freund's adjuvant, MPL™ and IL-12. In some embodiments, the vaccine compositions or nanoparticle immunogens disclosed herein (e.g., SARS-COV-2 vaccine composition) can be formulated as a controlled-release or time-release formulation. This can be achieved in a composition that contains a slow release polymer or via a microencapsulated delivery system or bioadhesive gel. The various pharmaceutical compositions can be prepared in accordance with standard procedures well known in the art. See, e.g., Remington's Pharmaceutical Sciences, 19 th Ed., Mack Publishing Company, Easton, Pa., 1995; Sustained and Controlled Release Drug Delivery Systems, J. R. Robinson, ed., Marcel Dekker, Inc., New York, 1978); U.S. Pat. Nos. 4,652,441 and 4,917,893; 4,677,191 and 4,728,721; and 4,675,189.

In one embodiment, the adjuvant is selected from the group consisting of Addavax, MF59, and a MPLA liposomal adjuvant.

Methods of formulating mRNA for known in the art, and include mRNA-lipid nanoparticle (LNP) compositions. For example, the LNPs in mRNA COVID-19 vaccines consist of four main components: a neutral phospholipid, cholesterol, a polyethylene-glycol (PEG)-lipid, and an ionizable cationic lipid. The latter contains positively charged ionizable amine groups (at low pH) to interact with the anionic mRNA during particle formation and also facilitate membrane fusion during internalization. In addition, PEG-lipid is used to control the particle size and act as a steric barrier to prevent aggregation during storage. Together with the mRNA, these components form particles of about 60-100 nm in size by using rapid mixing production techniques, and suitable formulations are discussed in Hou, X., Zaks, T., Langer, R. et al. Lipid nanoparticles for mRNA delivery. Nat Rev Mater (2021). https://doi.org/10.1038/s41578-021-00358-0.

In some embodiments, vaccine of the present disclosure comprises an RNA (e.g., mRNA) polynucleotide encoding a chimeric polypeptide as described herein. Embodiments of the present disclosure also provide combination RNA (e.g., mRNA) vaccines. A “combination RNA (e.g., mRNA) vaccine” of the present invention refers to a vaccine comprising at least one (e.g., at least 2, 3, 4, or 5) RNA (e.g., mRNA) polynucleotide having an open reading frame encoding a combination of any two or more (or all of) antigenic polypeptides of SARS-COV-2, in combination with an RNA encoding a chimeric polypeptide as described herein.

In one embodiment, the present invention provides a method for prophylaxis or treatment of a betacoronavirus infection in a subject comprising administering an effective amount of a vaccine composition as described herein.

As used herein, “treating” or “treatment” includes the reduction of any symptoms associated with COVID-19. The term includes therapeutic treatment as well as prophylactic or preventative measures to cure or halt or at least retard disease progress. Those in need of treatment include those already inflicted with a condition resulting from infection with SARS-COV-2 as well as those in which infection with SARS-COV-2 is to be prevented. Subjects partially or totally recovered form infection with SARS-COV-2 might also be in need of treatment. Prevention encompasses inhibiting or reducing the spread of SARS-COV-2 or inhibiting or reducing the onset, development or progression of one or more of the symptoms associated with infection with SARS-COV-2.

“Preventing” or “prevention” or “prophylaxis” includes the prevention of any symptoms associated with COVID-19 including the deterioration of the disease.

In another embodiment, the present invention provides a use of a chimeric polypeptide as described herein or a trimer thereof, or a nucleic acid as described herein, in the manufacture of a medicament for prophylaxis or treatment of SARS-COV-2 infection in a subject.

In one embodiment, the present invention provides a method for prophylaxis or treatment of a betacoronavirus infection in a subject comprising administering an effective amount of a vaccine composition as described herein.

In a preferred embodiment, a vaccine comprising a chimeric polypeptide as described herein or a trimer thereof, or a nucleic acid as described herein can be used to boost immunity (e.g. induce or increase an immune response against a SARS-COV-2 RBD) generated initially by currently licensed vaccine products (e.g. that do not comprise a chimeric polypeptide as described herein or a trimer thereof, or a nucleic acid as described herein).

In a preferred embodiment, a vaccine comprising a first chimeric polypeptide as described herein or a trimer thereof, or a nucleic acid as described herein can be used to minimise non-neutralising immune memory generated initially by currently licensed vaccine products (e.g. that do not comprise a chimeric polypeptide as described herein or a trimer thereof, or a nucleic acid as described herein).

Accordingly, in one embodiment, the subject is animal (e.g. human), that has been administered previously with a SARS-COV-2 vaccine that does not comprise a chimeric polypeptide as described herein or a trimer thereof, or a nucleic acid as described herein.

In a another embodiment, a vaccine comprising a second chimeric polypeptide as described herein or a trimer thereof, or a nucleic acid encoding a second chimeric polypeptide as described herein as described herein can be used to boost immunity (e.g. induce or increase an immune response against a SARS-COV-2 RBD) generated initially by a vaccine comprising a first chimeric polypeptide as described herein or a trimer thereof, or a nucleic acid encoding a first chimeric polypeptide as described herein.

In another embodiment, a vaccine comprising a second chimeric polypeptide as described herein or a trimer thereof, or a nucleic acid encoding a second chimeric polypeptide as described herein as described herein can be used to minimise non-neutralising immune memory generated initially by a vaccine comprising a first chimeric polypeptide as described herein or a trimer thereof, or a nucleic acid encoding a first chimeric polypeptide as described herein.

Accordingly, in one embodiment, the subject is animal (e.g. human), that has been administered previously with a vaccine comprising a chimeric polypeptide as described herein or a trimer thereof, or a nucleic acid encoding a chimeric polypeptide as described herein.

### EXAMPLES

### Example 1: Construction and Expression of a Chimeric Trimeric Receptor Binding Domain Vaccine

In light of existing and future variants of concern, the present inventors sought to develop a chimeric trimeric Receptor Binding Domain (CTR) vaccine comprising the spike RBD of SARS-COV-2 in an endemic betacoronavirus based scaffold (HKU-1).

In brief, the CTR platform is based upon a pre-fusion stabilised trimeric spike scaffold derived from endemic human coronavirus HKU1, into which the heterologous RBD of SARS-COV-2 has been engineered (CTR-WT; FIG. 1).

The chimeric HKU-1 spike/SARS-COV2 RBD proteins were synthesised as artificial genes (GeneArt) and cloned into a mammalian expression plasmid carrying a CMV IE promoter and BGH polyadenylation signal. The resultant DNA plasmids were grown in E. coli DH5a (Thermofisher) and purified using MAXIprep (Qiagen). Proteins were expressed using transient transfection of Expi293 or ExpiCHO cells (Thermofisher) and purified from supernatants using Nickel-NTA affinity chromatography (Cytiva) and size-exclusion chromatography using a Superose 6 16/70 column (Cytiva).

The amino acid sequence of the polypeptide used to generate the CT-WT vaccine is provided below

MFLIIFILPTTLAVIGDFNCTNSFINDYNKTIPRISEDVVDVSLGLGTYYVLNR
VYLNTTLLFTGYFPKSGANFRDLALKGSIYLSTLWYKPPFLSDFNNGIFSKVKNTKLY
VNNTLYSEFSTIVIGSVFVNTSYTIVVQPHNGILEITACQYTMCEYPHTVCKSKGSIRN
ESWHIDSSEPLCLFKKNFTYNVSADWLYFHFYQERGVFYAYYADVGMPTTFLFSLYL
GTILSHYYVMPLTCNAISSNTDNETLEYWVTPLSRRQYLLNFDEHGVITNAVDCSSSF
LSEIQCKTQSFAPNTGVYDLSGFTVQPTESIVRFPNITNLCPFGEVFNATRFASVYA
WNRKRISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYLYRLFRKSNLKPF
ERDISTEIYQAGSTPCNGVEGFNCYFPLQSYGFQPTNGVGYQPYRVVVLSFELLHA
PATVCGPKKSTNLVKNKCVNYDLYGITGQGIFKEVSAAYYNNWQNLLYDSNGNIIGF
KDFLTNKTYTILPCYSGRVSAAFYQNSSSPALLYRNLKCSYVLNNISFISQPFYFDSYL
GCVLNAVNLTSYSVSSCDLRMGSGFCIDYALPSSRASVGGISSPYRFVTFEPFNVSF
VNDSVETVGGLFEIQIPTNFTIAGHEEFIQTSSPKVTIDCSAFVCSNYAACHDLLSEYG
TFCDNINSILNEVNDLLDITQLQVANALMQGVTLSSNLNTNLHSDVDNIDFKSLLGCL
GSQCGSSSRSPLEDLLFNKVKLSDVGFVEAYNNCTGGSEIRDLLCVQSFNGIKVLPP
ILSETQISGYTTAATVAAMFPPWSAAAGVPFPLNVQYRINGLGVTMDVLNKNQKLIAN
AFNKALLSIQNGFTATPSALAKIQSVVNANAQALNSLLQQLFNKFGAISSSLQEILSRL
DPPEAQVQIDRLINGRLTALNAYVSQQLSDITLIKAGASRAIEKVNECVKSQSPRINFC
GNGNHILSLVQNAPYGLLFIHFSYKPTSFKTVLVSPGLCLSGDRGIAPKQGYFIKQND
SWMFTGSSYYYPEPISDKNVVFMNSCSVNFTKAPFIYLNNSIPNLSDFEAELSLWFK
NHTSIAPNLTFNSHINATFLDLYYEMNVIQESIKSLNSSFINLKEIGTYEMGSGYIPEAP
RDGQAYVRKDGEWVLLSTFLGSGLNDIFEAQKIEWHEGHHHHHH

HKU-1 sequence shown in plain text, SARS-COV-2 spike RBD sequence shown in bold, T4 fibritin Foldon shown in underlined italics, Avitag shown in bold underline, Polyhistidine tag shown in italics.

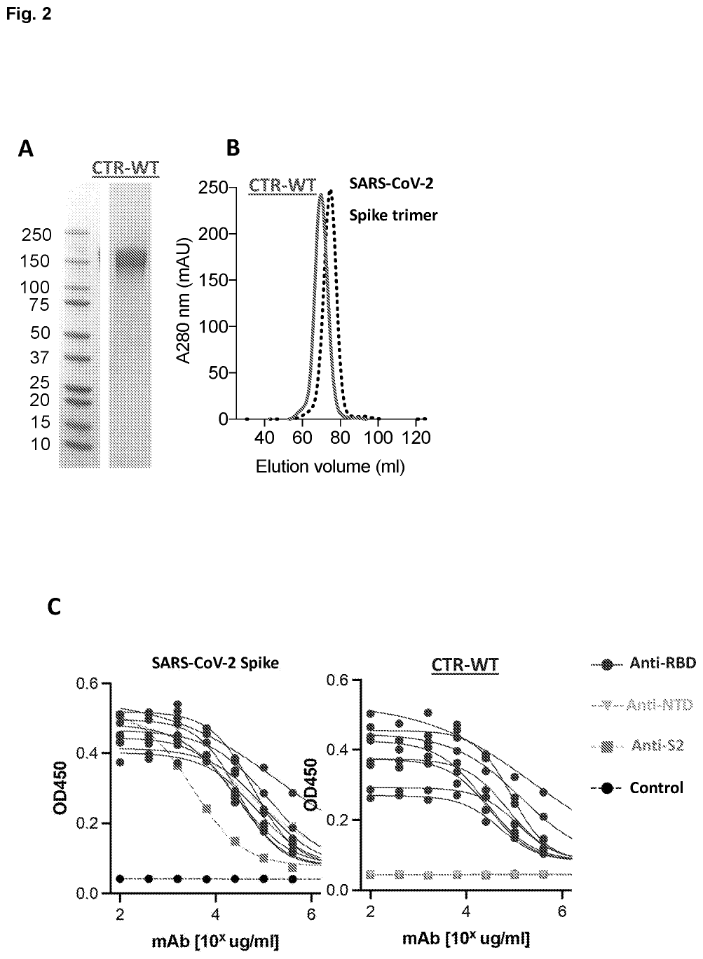

FIGS. 2a and b demonstrate that the chimeric polypeptides described herein expressed with high yields in mammalian cell expression systems and formed stable trimers (chimeric trimeric RBD) as assessed by size exclusion chromatography.

The present inventors have also demonstrated CTRs with a B.1.351 SARS-CoV-2 spike RBD and a B.1.617.2 SARS-COV-2 spike RBD can be expressed in mammalian cells using a HKI1 scaffold (data not shown).

### Example 2: Chimeric Trimeric Receptor Binding Domain Vaccine is Antigenically Intact

To confirm the Chimeric Trimeric Receptor Binding Domain vaccine is antigenically intact, ELISA was performed using a panel of 8 human mAbs binding key neutralising epitopes on the SARS-COV-2 RBD, including irivimab and imdevimab (Regeneron).

In brief, 96-well Maxisorp plates (Thermo Fisher) were coated overnight at 4° C. with 2 μg/mL recombinant CTR proteins. After blocking with 1% FCS in PBS, human monoclonal antibodies were serially diluted in PBS and added for two hours. Plates were then washed prior to incubation with 1:20000 dilution of HRP-conjugated anti-human IgG (Sigma) for 1 hour. Plates were washed and developed using TMB substrate (Sigma), stopped using 0.16 M sulphuric acid and read at 450 nm. Effective concentration midpoints (EC50) concentrations were calculated using a fitted curve (4 parameter log regression) and Prism 9.0 software (Graphpad).

FIG. 2c demonstrates that human anti-S2 and anti-NTD antibodies that bind to SARS-COV-2 spike do not bind the CTD vaccine, whereas human anti-RBD antibodies that bind to SARS-COV-2 spike are able to bind the CTD vaccine, indicating the RBD is antigenically intact.

### Example 3: Chimeric Trimeric Receptor Binding Domain Vaccine Induces Neutralising and Anti-RBD Antibodies In Vivo, and at Higher Titres than Convalescent Humans Following COVID-19 Infection

The ability of the Chimeric Trimeric Receptor Binding Domain vaccine to induce anti-RBD and neutralising antibodies was examined.

In brief, mice were immunised intramuscularly with two doses (21 days apart) of CTR-WT formulated with Addavax adjuvant (analogous to MF59). Two weeks after the booster dose, mice demonstrated high RBD IgG titres and serum neutralising activity assessed using gold-standard microneutralisation assays.

### Immunisations in Mice

Five micrograms of SARS-COV-2 spike or CTR-WT or OVA proteins were formulated in PBS at a 1:1 ratio with Addavax adjuvant (InvivoGen). C57BL/6 mice were anesthetised by isoflurane inhalation prior to intramuscular injection of 50 μL vaccine in each hind quadriceps. Primary responses were assessed 14 or 28 days after prime immunisation. Booster immunisations were administered 3 weeks post prime, and responses assessed 14 or 28 days after boost.

### Immunisations in NHP

Pigtail macaques (Macaca nemestrina) were housed in the Monash Animal Research Platform. 10 male macaques (Macaca nemestrina) (6-15 years old) were vaccinated with 25 μg of CTR-WT spike formulated with 100 μg of Monophosphoryl Lipid A (MPLA) liposomes (Polymun) 22 intramuscularly in the right quadriceps. Twenty-eight days after priming, booster immunisations consisting of 25 μg CTR-WT with 100 μg of MPLA and 1% tattoo ink were administered intramuscularly in both quadriceps. Macaques were necropsied 14 days after booster vaccine administration.

Microneutralisation Assay with ELISA-Based Read Out

Wildtype SARS-COV-2 (CoV/Australia/VIC/01/2020) and B.1.351 (CoV/Australia/QLD/1520/2020) isolates were passaged in Vero cells and stored at −80° C. 96-well flat bottom plates were seeded with Vero cells (20,000 cells per well in 100 μl). The next day, Vero cells were washed once with 200 μl serum-free DMEM and added with 150 μl of infection media (serum-free DMEM with 1.33 μg/ml TPCK trypsin). 2-fold serial dilutions of mAbs (from 5 μg/ml) were incubated with WT and B.1.351 SARS-COV-2 isolates at 2000 TCID₅₀/ml at 37° C. for 1 hour. Next, mAb-virus mixtures (50 μl) were added to Vero cells in duplicate and incubated at 37° C. for 48 hours. ‘Cells only’ and ‘virus+cells’ controls were included to represent 0% and 100% infectivity respectively. After 48 hours, all cell culture media were carefully removed from wells and 200 μl of 4% formaldehyde was added to fix the cells for 30 mins at room temperature. The plates were then dunked in a 1% formaldehyde bath for 30 minutes to inactivate any residual virus prior to removal from the BSL3 facility. Cells were washed once in PBS and then permeabilised with 150 μl of 0.1% Triton-X for 15 minutes. Following one wash in PBS, wells were blocked with 200 μl of blocking solution (4% BSA with 0.1% Tween-20) for 1 hour. After three washes in PBST (PBS with 0.05% Tween-20), wells were added with 100 μl of rabbit polyclonal anti-SARS-COV N antibody (Rockland, #200-401-A50) at a 1:8000 dilution in dilution buffer (PBS with 0.2% Tween-20, 0.1% BSA and 0.5% NP-40) for 1 hour. Plates were then washed six times in PBST and added with 100 μl of goat anti-rabbit IgG (Abcam, #ab6721) at a 1:8000 dilution for 1 hour. After six washes in PBST, plates were developed with TMB and stopped with 0.15M H₂SO₄. OD values read at 450 nm were then used to calculate % neutralisation with the following formula: (‘Virus+ cells’−‘sample’)÷(‘Virus+cells’−‘Cells only’)×100. IC50 values were determined using four-parameter nonlinear regression in GraphPad Prism with curve fits constrained to have a minimum of 0% and maximum of 100% neutralisation.

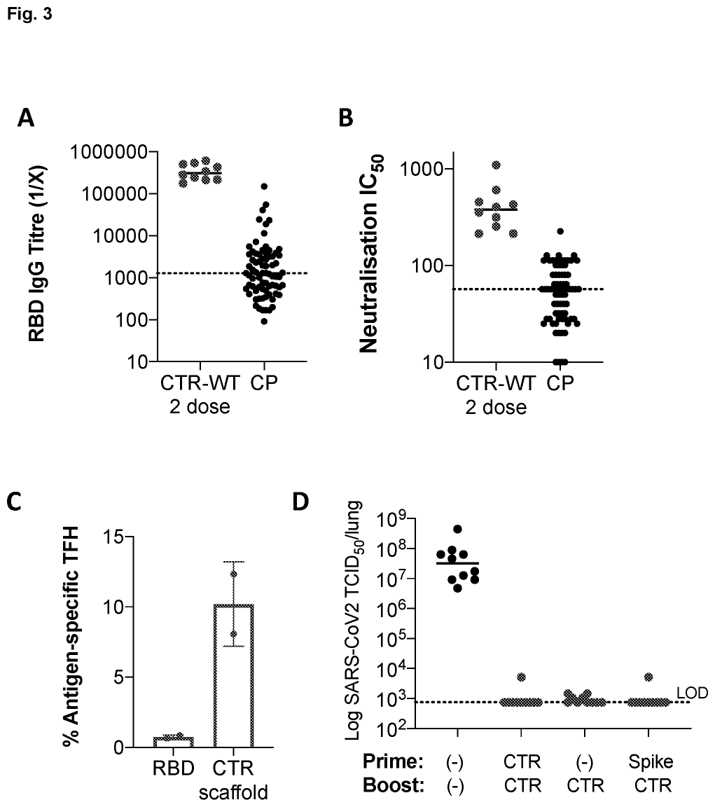

FIG. 3a demonstrates that anti-RBD antibody titre following immunisation with Chimeric Trimeric Receptor Binding Domain vaccine was substantially higher than levels observed in convalescent humans following COVID-19 infection.

Importantly, FIG. 3b demonstrates that neutralising antibody titre following immunisation with Chimeric Trimeric Receptor Binding Domain vaccine was substantially higher than levels observed in convalescent humans following COVID-19 infection, a level that predicts high efficacy in human trials.

### Example 4: Chimeric Trimeric Receptor Binding Domain Vaccine Induces Robust CD4 T Follicular Helper Cell Responses In Vivo

The ability of the Chimeric Trimeric Receptor Binding Domain vaccine to induce CD4 T follicular helper cell responses was examined.

Using a previously published assay (Jiang et al. (2019) J Immunol Methods 267:48-57), we demonstrated that T follicular helper cells elicited by the CTR vaccine predominately recognise the trimeric scaffold and not the RBD. Briefly, cells were isolated from vaccine-draining lymph nodes of mice immunised with CTR-WT, and cultured with overlapping peptide pools spanning the human HKU1 Spike protein or the SARS-COV-2 RBD. 18 hours later, cells were stained and analysed the expression of activation markers in peptide stimulated cultures compared to negative control wells. Robust T follicular helper cell responses were elicited to the HKU1 peptide pool, with minimal responses directed toward the SARS-COV-2 RBD.

Importantly, this confirms that the CTR protein scaffold provides T cell epitopes that are lacking in the RBD, which can support the antibody response to the vaccine.

FIG. 3c demonstrates that relative to the poor levels of CD4 T cell help elicited by the RBD alone, the Chimeric Trimeric Receptor Binding Domain vaccine induces robust CD4 T follicular helper cell responses to support antibody production.

### Example 5: Chimeric Trimeric Receptor Binding Domain Vaccine Induces Near Complete Suppression of Viral Replication in the Lungs of Mice

The protective capacity of the Chimeric Trimeric Receptor Binding Domain vaccine was examined using a murine model of SARS-COV-2 N501Y variant virus infection.

In brief, mice were immunised with one or two doses of CTR-WT, or administered a single dose of SARS-COV-2 spike protein (modelling prior immunisation in humans) followed by a single dose of CTR-WT. Mice were challenged with aerosolised SARS-COV-2 N501Y at 7 weeks after the last immunisation. C57BL/6J mice were immunised as above with either one or two doses of CTR-WT, or a single dose of spike followed by a single dose of CTR-WT. Immunised mice were subsequently transferred to an OGTR-approved Physical Containment Level 3 (PC-3) facility at the Walter and Eliza Hall Institute of Medical Research (Cert-3621; IA88_20). After acclimatisation, mice were subject to SARS-COV-2 infection (clinical isolate hCoV-19/Australia/VIC2089/2020) using an inhalation exposure system (Glas-Col, LLC) for 45 minutes loaded with 1.5×10⁷ SARS-COV-2 TCID50. Three days post-infection, animals were humanely killed and lungs removed and homogenised in a Bullet Blender (Next Advance Inc) in 1 mL DME media (ThermoFisher) containing steel homogenisation beads (Next Advance Inc). Samples were clarified by centrifugation at 10,000×g for 5 minutes before virus quantification by TCID50 assays. SARS-COV-2 live virus quantification by TCID50 assay: SARS-COV-2 lung TCID50 was determined by plating 1:7 serially-diluted lung tissue homogenate onto confluent layers of Vero cells (clone CCL81) in DME media (ThermoFisher) containing 0.5 μg/ml trypsin-TPCK (ThermoFisher) in replicates of six on 96-well plates. Plates were incubated at 37° C. supplied with 5% CO₂ for four days before measuring cytopathic effect under light microscope. The TCID50 calculation was performed using the Spearman and Kärber method.

FIG. 3d demonstrates that in all cases, a near complete suppression of viral replication in the lungs of immunised mice (>6 log reduction) was observed, including durable sterilising protection after a single CTR-WT injection (FIG. 3d).

### Example 6: Chimeric Trimeric Receptor Binding Domain Vaccine Induces Neutralising and Anti-RBD Antibodies in Non-Human Primates, and at Higher Titres than Convalescent Humans Following COVID-19 Infection

The ability of the Chimeric Trimeric Receptor Binding Domain vaccine to induce anti-RBD and neutralising antibodies was examined.

In brief, 5 pigtail macaques were immunised intramuscularly with two doses 28 days apart of CTR-WT formulated with a clinical trial grade MPLA liposomal adjuvant (supplied by Al Katinger at Polymun), with immunogenicity assessed 14 days after the booster dose.

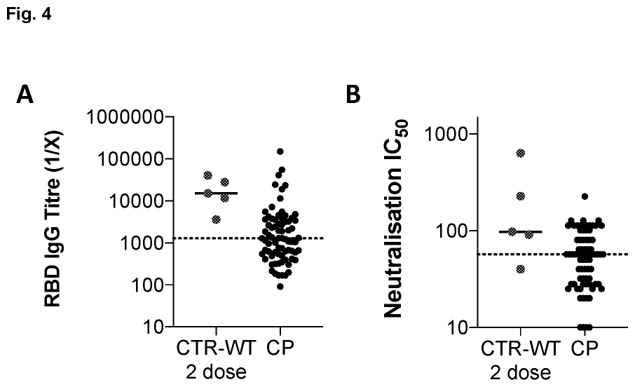

FIG. 4 shows Immunised macaques exhibited potent RBD-specific IgG and serum neutralisation titres (11.8 and 1.7-fold higher than median levels of convalescent human subjects respectively).

To model the relationship between in vitro neutralization levels and the observed protection from severe acute respiratory syndrome coronavirus 2 (SARS-CoV-2) infection, neutralization titers were determined for each study and used to determine the standard deviation of the log 10-transformed neutralization titers for each study. To model the relationship between the neutralization level of antibodies collected from individuals after vaccination (or during convalescence) and the protection from COVID-19 a logistic relationship between neutralization level and protective efficacy was assumed, and models of protection fitted to the data as per Khoury, D. S. et al. Neutralizing antibody levels are highly predictive of immune protection from symptomatic SARS-COV-2 infection. Nat Med 27, 1205-1211 (2021),

Without wishing to be bound by theory, these neutralising titres are at a level that modelling indicates would be associated with robust (>90%) protective efficacy based on current COVID-19 vaccines.

These data also indicate the safety and strong immunogenicity of the CTR-WT platform in non-human primates

### Example 7: Chimeric Trimeric Receptor Binding Domain Vaccine Induces Neutralising and Anti-RBD Antibodies in Non-Human Primates, and at Higher Titres than Convalescent Humans Following COVID-19 Infection

The ability of the Chimeric Trimeric Receptor Binding Domain vaccine to induce anti-RBD and neutralising antibodies is examined using Chimeric Trimeric Receptor Binding Domain vaccines comprising a spike RBD from a beta variant of SARS-COV-2 (comprising N501Y, E484K, K417N mutations from VOC B.1.351), a spike RBD from a delta variant of SARS-COV-2 (comprising T478K and L452R mutations from VOC B.1.617.2), CTR-5mut (comprising N501Y, E484K, K417N, T478K and L452R mutations) or CTR-5mut 2.0 (comprising N501Y, E484K, K417N, S477N and L452R mutations).

In brief, CTR vaccines formulated with MF59 are tested using single and two dose regimes in both naïve and pre-immune contexts using wildtype and hACE2 transgenic mouse models. Protective efficacy in mice will be established by viral challenge with Wuhan-like and VOC SARS-COV-2 viruses.

Syrian hamster models, which develop pathology in response to SARS-COV-2 infection are used to assess efficacy against VOC.

CTR vaccine immunogenicity and dosing is assessed in macaques, both in naïve and animals pre-immunised with first generation COVID-19 vaccines, antibody, B and T cell immunity examined, and at necropsy animal organs harvested for histopathology for indicative toxicological analysis.

### Example 8: Construction and Expression of Chimeric Trimeric Omicron Receptor Binding Domain Vaccines, Including Using Scaffolds from Different Equine Coronavirus and Mouse Hepatitis Virus

In light of existing and future variants of concern, the present inventors prepared further chimeric trimeric Receptor Binding Domain (CTR) vaccine comprising the spike RBD of Omicron variants of SARS-COV-2 in an endemic betacoronavirus based scaffold (HKU-1).

In brief, a chimeric HKU-1 spike/SARS-COV2 RBD proteins were synthesised and expressed as per Example 1.

The amino acid sequence of the polypeptide used to generate the CT-Omicron BA.1 vaccine is provided below:

MFLIIFILPTTLAVIGDFNCTNSFINDYNKTIPRISEDVVDVSLGLGTYYVLNR
VYLNTTLLFTGYFPKSGANFRDLALKGSIYLSTLWYKPPFLSDFNNGIFSKVKNTKLY
VNNTLYSEFSTIVIGSVFVNTSYTIVVQPHNGILEITACQYTMCEYPHTVCKSKGSIRN
ESWHIDSSEPLCLFKKNFTYNVSADWLYFHFYQERGVFYAYYADVGMPTTFLFSLYL
GTILSHYYVMPLTCNAISSNTDNETLEYWVTPLSRRQYLLNFDEHGVITNAVDCSSSF
LSEIQCKTQSFAPNTGVYDLSGFTVQPTESIVRFPNITNLCPFDEVFNATRFASVYA
WNRKRISNCVADYSVLYNLAPFFTFKCYGVSPTKLNDLCFTNVYADSFVIRGDEVR
QIAPGQTGNIADYNYKLPDDFTGCVIAWNSNKLDSKVSGNYNYLYRLFRKSNLKPF
ERDISTEIYQAGNKPCNGVAGFNCYFPLRSYSFRPTYGVGHQPYRVVVLSFELLHA
PATVCGPKKSTNLVKNKCVNYDLYGITGQGIFKEVSAAYYNNWQNLLYDSNGNIIGF
KDFLTNKTYTILPCYSGRVSAAFYQNSSSPALLYRNLKCSYVLNNISFISQPFYFDSYL
GCVLNAVNLTSYSVSSCDLRMGSGFCIDYALPSSRASVGGISSPYRFVTFEPFNVSF
VNDSVETVGGLFEIQIPTNFTIAGHEEFIQTSSPKVTIDCSAFVCSNYAACHDLLSEYG
TFCDNINSILNEVNDLLDITQLQVANALMQGVTLSSNLNTNLHSDVDNIDFKSLLGCL
GSQCGSSSRSPLEDLLFNKVKLSDVGFVEAYNNCTGGSEIRDLLCVQSFNGIKVLPP
ILSETQISGYTTAATVAAMFPPWSAAAGVPFPLNVQYRINGLGVTMDVLNKNQKLIAN
AFNKALLSIQNGFTATPSALAKIQSVVNANAQALNSLLQQLFNKFGAISSSLQEILSRL
DPPEAQVQIDRLINGRLTALNAYVSQQLSDITLIKAGASRAIEKVNECVKSQSPRINFC
GNGNHILSLVQNAPYGLLFIHFSYKPTSFKTVLVSPGLCLSGDRGIAPKQGYFIKQND
SWMFTGSSYYYPEPISDKNVVFMNSCSVNFTKAPFIYLNNSIPNLSDFEAELSLWFK
NHTSIAPNLTFNSHINATFLDLYYEMNVIQESIKSLNSSFINLKEIGTYEMGSGYIPEAP
RDGQAYVRKDGEWVLLSTFLGSGLNDIFEAQKIEWHEGHHHHHH

HKU-1 sequence shown in plain text, Omicron BA.1 SARS-COV-2 spike RBD sequence shown in bold, T4 fibritin Foldon shown in underlined italics, Avitag shown in bold underline, Polyhistidine tag shown in italics.

A second BA.1 version has been generated using an alternative linker between the foldon sequence and the polyhistidine tag:

GSGYIPEAPRDGQAYVRKDGEWVLLSTFLGGGGSGGGGSGGGGSHHHH
HH

(Foldon shown in underlined italics, Polyhistidine tag shown in italics).

The amino acid sequence of the polypeptide used to generate the CT-Omicron BA.2 vaccine is provided below:

MFLIIFILPTTLAVIGDFNCTNSFINDYNKTIPRISEDVVDVSLGLGTYYVLNR
VYLNTTLLFTGYFPKSGANFRDLALKGSIYLSTLWYKPPFLSDFNNGIFSKVKNTKLY
VNNTLYSEFSTIVIGSVFVNTSYTIVVQPHNGILEITACQYTMCEYPHTVCKSKGSIRN
ESWHIDSSEPLCLFKKNFTYNVSADWLYFHFYQERGVFYAYYADVGMPTTFLFSLYL
GTILSHYYVMPLTCNAISSNTDNETLEYWVTPLSRRQYLLNFDEHGVITNAVDCSSSF
LSEIQCKTQSFAPNTGVYDLSGFTVQPTESIVRFPNITNLCPFDEVFNATRFASVYAW
NRKRISNCVADYSVLYNFAPFFAFKCYGVSPTKLNDLCFTNVYADSFVIRGNEVSQIA
PGQTGNIADYNYKLPDDFTGCVIAWNSNKLDSKVGGNYNYLYRLFRKSNLKPFERDI
STEIYQAGNKPCNGVAGFNCYFPLRSYGFRPTYGVGHQPYRVVVLSFELLHAPATV
CGPKKSTNLVKNKCVNYDLYGITGQGIFKEVSAAYYNNWQNLLYDSNGNIIGFKDFL
TNKTYTILPCYSGRVSAAFYQNSSSPALLYRNLKCSYVLNNISFISQPFYFDSYLGCV
LNAVNLTSYSVSSCDLRMGSGFCIDYALPSSRASVGGISSPYRFVTFEPFNVSFVND
SVETVGGLFEIQIPTNFTIAGHEEFIQTSSPKVTIDCSAFVCSNYAACHDLLSEYGTFC
DNINSILNEVNDLLDITQLQVANALMQGVTLSSNLNTNLHSDVDNIDFKSLLGCLGSQ
CGSSSRSPLEDLLFNKVKLSDVGFVEAYNNCTGGSEIRDLLCVQSFNGIKVLPPILSE
TQISGYTTAATVAAMFPPWSAAAGVPFPLNVQYRINGLGVTMDVLNKNQKLIANAFN
KALLSIQNGFTATPSALAKIQSVVNANAQALNSLLQQLFNKFGAISSSLQEILSRLDPP
EAQVQIDRLINGRLTALNAYVSQQLSDITLIKAGASRAIEKVNECVKSQSPRINFCGNG
NHILSLVQNAPYGLLFIHFSYKPTSFKTVLVSPGLCLSGDRGIAPKQGYFIKQNDSWM
FTGSSYYYPEPISDKNVVFMNSCSVNFTKAPFIYLNNSIPNLSDFEAELSLWFKNHTSI
APNLTENSHINATFLDLYYEMNVIQESIKSLNSSFINLKEIGTYEMGSGYIPEAPRDGQ
AYVRKDGEWVLLSTFLGSGLNDIFEAQKIEWHEGHHHHHH

HKU-1 sequence shown in plain text, Omicron BA.2 SARS-COV-2 spike RBD sequence shown in bold, T4 fibritin Foldon shown in underlined italics, Avitag shown in bold underline, Polyhistidine tag shown in italics.

A second BA.2 version has been generated using an alternative linker between the foldon sequence and the polyhistidine tag:

GSGYIPEAPRDGQAYVRKDGEWVLLSTFLGGGGSGGGGSGGGGSHHHH
HH

(Foldon shown in underlined italics, Polyhistidine tag shown in italics).

The present inventors prepared further chimeric trimeric Receptor Binding Domain (CTR) vaccines comprising the spike RBD of Omicron variants of SARS-COV-2 in an equine coronavirus (ECoV) scaffold.

In brief, the chimeric ECOV spike/SARS-COV2 RBD proteins were synthesised and expressed as per Example 1, including

The amino acid sequence of the polypeptide used to generate the CT-Omicron BA.2 vaccine is provided below:

MFLILLISLPTAFAVIGDLKCTTVSINDVDTGVPSISTDTVDVTNGLGTYYVL
DRVYLNTTLLLNGYYPTSGSTYRNMALKGTLLLSTLWFKPPFLSDFTNGIFAKVKNTK
VIKDGVMYSEFPAITIGSTFVNTSYSVVVQPHTTILGNKLQGFLEISVCQYTMCEYPNT
ICNPNLGNQRVELWHWDTGVVSCLYKRNFTYDVNADYLYFHFYQEGGTFYAYFTDT
GVVTKFLFNVYLGTVLSHYYVMPLTCNSALTLEYWVTPLTSKQYLLAFNQDGVIFNA
VDCKSDFMSEIKCKTLSIAPSTGVYELNGYTVQPTESIVRFPNITNLCPFDEVFNATRF
ASVYAWNRKRISNCVADYSVLYNFAPFFAFKCYGVSPTKLNDLCFTNVYADSFVIRG
NEVSQIAPGQTGNIADYNYKLPDDFTGCVIAWNSNKLDSKVGGNYNYLYRLFRKSNL
KPFERDISTEIYQAGNKPCNGVAGFNCYFPLRSYGFRPTYGVGHQPYRVVVLSFELL
HAPATVCGPKKSTNLVKNKCVNYDLYGITGQGIFVEVNATYYNSWQNLLYDSNGNL
YGFRDYLTNRTFMIRSCYSGRVSAAFHANSSEPALLFRNIKCNYVFNNTLSRQLQPI
NYFDSYLGCVVNADNSTSSVVQTCDLTVGSGYCVDYSTKSRASGSITTGYRFTNFE
PFTVNSVNDSLEPVGGLYEIQIPSEFTIGNMEEFIQTSSPKVTIDCSAFVCGDYAACK
SQLVEYGSFCDNINAILTEVNELLDTTQLQVANSLMNGVTLSTKLKDGVNFNVDDINF
SPVLGCLGSDCNKVSSRSPIEDLLFSKVKLSDVGFVEAYNNCTGGAEIRDLICVQSY
NGIKVLPPLLSENQISGYTLAATSASLFPPWSAAAGVPFPLNVQYRINGIGVTMDVLS
QNQKLIANAFNNALGAIQEGFDATPSALVKIQAVVNANAEALNNLLQQLSNRFGAISS
SLQEILSRLDPPEAQAQIDRLINGRLTALNAYVSQQLSDSTLVKFSAAQAMEKVNECV
KSQSSRINFCGNGNHIISLVQNAPYGLYFIHFSYVPTKYVTAKVSPGLCIAGDRGIAPK
SGYFVNVNNTWMFTGSGYYYPEPITGNNVVVMSTCAVNYTKAPDVMLNISTPNLPY
FKEELDQWFKNQTSVAPDLSLDYINVTFLDLQDEMNRLQEAIKVLNQSYINLKDIGTY
EYYGSGYIPEAPRDGQAYVRKDGEWVLLSTFLGSGLNDIFEAQKIEWHEGHHHHHH

ECoV sequence shown in plain text, Omicron BA.2 SARS-COV-2 spike RBD sequence shown in bold, T4 fibritin Foldon shown in underlined italics, Avitag shown in bold underline, Polyhistidine tag shown in italics.

A second BA.2 ECoV vaccine has been generated using an alternative linker between the foldon sequence and the polyhistidine tag:

GSGYIPEAPRDGQAYVRKDGEWVLLSTFLGGGGSGGGGSGGGGSHHHH
HH

(Foldon shown in underlined italics, Polyhistidine tag shown in italics).

A third BA.2 ECoV vaccine has been generated using the RBD of the CTR-WT vaccine of Example 1.

The present inventors prepared further chimeric trimeric Receptor Binding Domain (CTR) vaccines comprising the spike RBD of Omicron variants of SARS-COV-2 in a mouse hepatitis (MHV) scaffold.

In brief, the chimeric MHV spike/SARS-COV2 RBD proteins were synthesised and expressed as per Example 1.

The amino acid sequence of the polypeptide used to generate the CT-Omicron BA.2 vaccine is provided below:

MLFVFILFLPSCLGYIGDFRCIQLVNSNGANVSAPSISTETVEVSQGLGTY
YVLDRVYLNATLLLTGYYPVDGSKFRNLALTGTNSVSLSWFQPPYLSQFNDGIFAKV
QNLKTSTPSGATAYFPTIVIGSLFGYTSYTVVIEPYNGVIMASVCQYTICQLPYTDCKP
NTNGNKLIGFWHTDVKPPICVLKRNFTLNVNADAFYFHFYQHGGTFYAYYADKPSAT
TFLFSVYIGDILTQYYVLPFICNPTAGSTFAPRYWVTPLVKRQYLFNFNQKGVITSAVD
CASSYTSEIKCKTQSMLPSTGVYELSGYTVQPTESIVRFPNITNLCPFDEVFNATRFA
SVYAWNRKRISNCVADYSVLYNFAPFFAFKCYGVSPTKLNDLCFTNVYADSFVIRGN
EVSQIAPGQTGNIADYNYKLPDDFTGCVIAWNSNKLDSKVGGNYNYLYRLFRKSNLK
PFERDISTEIYQAGNKPCNGVAGFNCYFPLRSYGFRPTYGVGHQPYRVVVLSFELLH
APATVCGPKKSTNLVKNKCVKYDLYGITGQGVFKEVKADYYNSWQTLLYDVNGNLN
GFRDLTTNKTYTIRSCYSGRVSAAFHKDAPEPALLYRNINCSYVFSNNISREENPLNY
FDSYLGCVVNADNRTDEALPNCDLRMGAGLCVDYSKSSRASGSVSTGYRLTTFEPY
TPMLVNDSVQSVDGLYEMQIPTNFTIGHHEEFIQTRSPKVTIDCAAFVCGDNTACRQ
QLVEYGSFCVNVNAILNEVNNLLDNMQLQVASALMQGVTISSRLPDGISGPIDDINFS
PLLGCIGSTCAEDGNGPSAIRGRSPIEDLLFDKVKLSDVGFVEAYNNCTGGQEVRDL
LCVQSFNGIKVLPPVLSESQISGYTTGATAAAMFPPWSAAAGVPFPLSVQYRINGLG
VTMNVLSENQKMIASAFNNALGAIQDGFDATPSALGKIQSVVNANAEALNNLLNQLS
NRFGAISASLQEILTRLEPPEAKAQIDRLINGRLTALNAYISKQLSDSTLIKVSAAQAIEK
VNECVKSQTTRINFCGNGNHILSLVQNAPYGLYFIHFSYVPISFTTANVSPGLCISGD
RGLAPKAGYFVQDDGEWKFTGSSYYYPEPITDKNSVIMSSCAVNYTKAPEVFLNTSI
PNPPDFKEELDKWFKNQTSIAPDLSLDFEKLNVTLLDLTYEMNRIQDAIKKLNESYINL
KEVGTYEMYGSGYIPEAPRDGQAYVRKDGEWVLLSTFLGSGLNDIFEAQKIEWHEG
HHHHHH

MHV sequence shown in plain text, Omicron BA.2 SARS-COV-2 spike RBD sequence shown in bold, T4 fibritin Foldon shown in underlined italics, Avitag shown in bold underline, Polyhistidine tag shown in italics.

A second BA.2 MHV vaccine has been generated using an alternative linker sequence and the polyhistidine tag: between the foldon

GSGYIPEAPRDGQAYVRKDGEWVLLSTFLGGGGSGGGGSGGGGSHHHH
HH

(Foldon shown in underlined italics, Polyhistidine tag shown in italics) in MHV.

A third BA.2 MHV vaccine has been generated using the RBD of the CTR-WT vaccine of Example 1.

The present inventors have demonstrated CTRs with the SARS-COV-2 spike RBD of the CTR WT vaccine of Example 1 can be expressed in mammalian cells using the ECoV and MHV scaffolds (data not shown).

The present inventors have also demonstrated CTRs with the SARS-COV-2 spike RBD of Omicron BA.2 can be expressed with high yields in mammalian cell expression systems and formed stable trimers (chimeric trimeric RBD) as assessed by size exclusion chromatography.

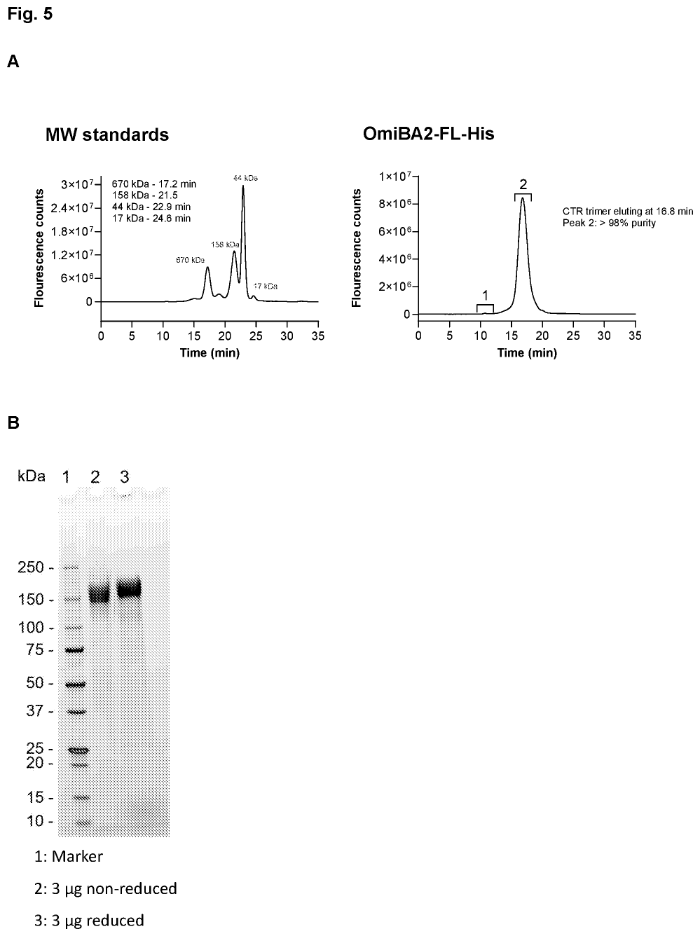

For example, FIG. 5 demonstrates that the CTRs with the SARS-COV-2 spike RBD of Omicron BA.2 in HKU-1 is expressed with high yields in mammalian cell expression systems and formed stable trimers (chimeric trimeric RBD) as assessed by size exclusion chromatography. In brief, expressed proteins were separated on Superose 6 10_30GL in 1×DPBS and detected using fluorescence (Em. 280 nm, Exc. 340 nm.

1. A chimeric polypeptide comprising an amino acid sequence encoding a N terminal domain of a first betacoronavirus spike protein, an amino acid sequence encoding one or more immunogenic epitope of a receptor binding domain of a second betacoronavirus spike protein, and an amino acid sequence encoding a C terminal region of the first coronavirus spike protein.

2. A chimeric polypeptide comprising an amino acid sequence encoding a N terminal domain of a first betacoronavirus spike protein, an amino acid sequence encoding a receptor binding domain of a second betacoronavirus spike protein, and an amino acid sequence encoding a C terminal region of the first coronavirus spike protein.

3. A chimeric polypeptide according to claim 1 or claim 2 wherein the amino acid sequence encoding the C terminal region of the first coronavirus spike protein encodes the S2′ protease cleavage site, fusion peptide, heptad repeat 1, central helix, connector domain and heptad repeat 2 regions of the first betacoronavirus spike protein.

4. A chimeric polypeptide according to any one of claims 1 to 3 wherein the second betacoronavirus spike protein is a SARS-COV, MERS-COV, or SARS-COV-2 betacoronavirus spike protein.

5. A chimeric polypeptide according to any one of claims 1 to 4 wherein the first betacoronavirus spike protein is selected from the group consisting of a HCoVHKU1 betacoronavirus spike protein, an ECOV betacoronavirus spike protein, and a MHV spike protein.

6. A chimeric polypeptide according to any one of claims 1 to 5 wherein the second betacoronavirus spike protein is selected from the group consisting of a B.1.1.7 (Alpha), B.1.351 (Beta), B.1.617.2 (Delta), P.1 (Gamma), B.1.427 (Epsilon), B.1.429 (Epsilon), B.1.525 (Eta), B.1.526 (Iota) B.1.617.1 (Kappa), B.1.617.3, C.37 (Lambda) and B.1.621 (Mu) spike protein.

7. A chimeric polypeptide according to any one of claims 1 to 6 wherein the chimeric polypeptide further comprises an amino acid sequence encoding a trimerization domain.

8. A chimeric polypeptide according to claim 7 wherein the trimerization domain is the C-terminal domain of T4 fibritin.

9. A chimeric polypeptide according to any one of claims 1 to 8 wherein the amino acid sequence encoding the N terminal domain is selected from the group consisting of:
(i)
MFLIIFILPTTLAVIGDFNCTNSFINDYNKTIPRISEDVVDVSLGLGTYYVLNRVYLNTTL
LFTGYFPKSGANFRDLALKGSIYLSTLWYKPPFLSDFNNGIFSKVKNTKLYVNNTLYS
EFSTIVIGSVFVNTSYTIVVQPHNGILEITACQYTMCEYPHTVCKSKGSIRNESWHIDS
SEPLCLFKKNFTYNVSADWLYFHFYQERGVFYAYYADVGMPTTFLFSLYLGTILSHY
YVMPLTCNAISSNTDNETLEYWVTPLSRRQYLLNFDEHGVITNAVDCSSSFLSEIQCK
TQSFAPNTGVYDLSGFTV
or
a variant thereof;
(ii)
MFLILLISLPTAFAVIGDLKCTTVSINDVDTGVPSISTDTVDVTNGLGTYYVLDRVYLNT
TLLLNGYYPTSGSTYRNMALKGTLLLSTLWFKPPFLSDFTNGIFAKVKNTKVIKDGVM
YSEFPAITIGSTFVNTSYSVVVQPHTTILGNKLQGFLEISVCQYTMCEYPNTICNPNLG
NQRVELWHWDTGVVSCLYKRNFTYDVNADYLYFHFYQEGGTFYAYFTDTGVVTKF
LFNVYLGTVLSHYYVMPLTCNSALTLEYWVTPLTSKQYLLAFNQDGVIFNAVDCKSD
FMSEIKCKTLSIAPSTGVYELNGYTV
or
a variant thereof;
and
(iii)
MLFVFILFLPSCLGYIGDFRCIQLVNSNGANVSAPSISTETVEVSQGLGTYYVLDRVYL
NATLLLTGYYPVDGSKFRNLALTGTNSVSLSWFQPPYLSQFNDGIFAKVQNLKTSTP
SGATAYFPTIVIGSLFGYTSYTVVIEPYNGVIMASVCQYTICQLPYTDCKPNTNGNKLI
GFWHTDVKPPICVLKRNFTLNVNADAFYFHFYQHGGTFYAYYADKPSATTFLFSVYI
GDILTQYYVLPFICNPTAGSTFAPRYWVTPLVKRQYLFNFNQKGVITSAVDCASSYTS
EIKCKTQSMLPSTGVYELSGYTV.

10. A chimeric polypeptide according to any one of claims 1 to 9 wherein the amino acid sequence encoding the C terminal region is selected from the group consisting of:
(i)
CVNYDLYGITGQGIFKEVSAAYYNNWQNLLYDSNGNIIGFKDFLINKTYTILPCYSGR
VSAAFYQNSSSPALLYRNLKCSYVLNNISFISQPFYFDSYLGCVLNAVNLTSYSVSSC
DLRMGSGFCIDYALPSSRASVGGISSPYRFVTFEPFNVSFVNDSVETVGGLFEIQIPT
NFTIAGHEEFIQTSSPKVTIDCSAFVCSNYAACHDLLSEYGTFCDNINSILNEVNDLLDI
TQLQVANALMQGVTLSSNLNTNLHSDVDNIDFKSLLGCLGSQCGSSSRSPLEDLLFN
KVKLSDVGFVEAYNNCTGGSEIRDLLCVQSFNGIKVLPPILSETQISGYTTAATVAAM
FPPWSAAAGVPFPLNVQYRINGLGVTMDVLNKNQKLIANAFNKALLSIQNGFTATPS
ALAKIQSVVNANAQALNSLLQQLFNKFGAISSSLQEILSRLDPPEAQVQIDRLINGRLT
ALNAYVSQQLSDITLIKAGASRAIEKVNECVKSQSPRINFCGNGNHILSLVQNAPYGL
LFIHFSYKPTSFKTVLVSPGLCLSGDRGIAPKQGYFIKQNDSWMFTGSSYYYPEPISD
KNVVFMNSCSVNFTKAPFIYLNNSIPNLSDFEAELSLWFKNHTSIAPNLTENSHINATF
LDLYYEMNVIQESIKSLNSSFINLKEIGTYEM
or
a variant thereof;
(ii)
CVNYDLYGITGQGIFVEVNATYYNSWQNLLYDSNGNLYGFRDYLTNRTFMIRSCYS
GRVSAAFHANSSEPALLFRNIKCNYVFNNTLSRQLQPINYFDSYLGCVVNADNSTSS
WVQTCDLTVGSGYCVDYSTKSRASGSITTGYRFTNFEPFTVNSVNDSLEPVGGLYEI
QIPSEFTIGNMEEFIQTSSPKVTIDCSAFVCGDYAACKSQLVEYGSFCDNINAILTEVN
ELLDTTQLQVANSLMNGVTLSTKLKDGVNFNVDDINFSPVLGCLGSDCNKVSSRSPI
EDLLFSKVKLSDVGFVEAYNNCTGGAEIRDLICVQSYNGIKVLPPLLSENQISGYTLAA
TSASLFPPWSAAAGVPFPLNVQYRINGIGVTMDVLSQNQKLIANAFNNALGAIQEGF
DATPSALVKIQAVVNANAEALNNLLQQLSNRFGAISSSLQEILSRLDPPEAQAQIDRLI
NGRLTALNAYVSQQLSDSTLVKFSAAQAMEKVNECVKSQSSRINFCGNGNHIISLVQ
NAPYGLYFIHFSYVPTKYVTAKVSPGLCIAGDRGIAPKSGYFVNVNNTWMFTGSGYY
YPEPITGNNVVVMSTCAVNYTKAPDVMLNISTPNLPYFKEELDQWFKNQTSVAPDLS
LDYINVTFLDLQDEMNRLQEAIKVLNQSYINLKDIGTYEYY
or
a variant thereof;
and
(iii)
CVKYDLYGITGQGVFKEVKADYYNSWQTLLYDVNGNLNGFRDLTTNKTYTIRSCYS
GRVSAAFHKDAPEPALLYRNINCSYVFSNNISREENPLNYFDSYLGCVVNADNRTDE
ALPNCDLRMGAGLCVDYSKSSRASGSVSTGYRLTTFEPYTPMLVNDSVQSVDGLYE
MQIPTNFTIGHHEEFIQTRSPKVTIDCAAFVCGDNTACRQQLVEYGSFCVNVNAILNE
VNNLLDNMQLQVASALMQGVTISSRLPDGISGPIDDINFSPLLGCIGSTCAEDGNGPS
AIRGRSPIEDLLFDKVKLSDVGFVEAYNNCTGGQEVRDLLCVQSFNGIKVLPPVLSES
QISGYTTGATAAAMFPPWSAAAGVPFPLSVQYRINGLGVTMNVLSENQKMIASAFN
NALGAIQDGFDATPSALGKIQSVVNANAEALNNLLNQLSNRFGAISASLQEILTRLEP
PEAKAQIDRLINGRLTALNAYISKQLSDSTLIKVSAAQAIEKVNECVKSQTTRINFCGN
GNHILSLVQNAPYGLYFIHFSYVPISFTTANVSPGLCISGDRGLAPKAGYFVQDDGE
WKFTGSSYYYPEPITDKNSVIMSSCAVNYTKAPEVFLNTSIPNPPDFKEELDKWFKN
QTSIAPDLSLDFEKLNVTLLDLTYEMNRIQDAIKKLNESYINLKEVGTYEMY

11. A chimeric polypeptide according to any one of claims 1 to 10 wherein the amino acid sequence encoding the RBD domain is selected from the group consisting of:
QPTESIVRFPNITNLCPFGEVFNATRFASVYAWNRKRISNCVADYSVLY
NSASFSTFKCYGVSPTKLNDLCFTNVYADSFVIRGDEVRQIAPGQTGKI
ADYNYKLPDDFTGCVIAWNSNNLDSKVGGNYNYLYRLFRKSNLKPFERD
ISTEIYQAGSTPCNGVEGFNCYFPLQSYGFQPTNGVGYQPYRVVVLSFE
LLHAPATVCGPKKSTNLVKNK
or
a variant thereof;
QPTESIVRFPNITNLCPFGEVFNATRFASVYAWNRKRISNCVADYSVLY
NSASFSTFKCYGVSPTKLNDLCFTNVYADSFVIRGDEVRQIAPGQTGNI
ADYNYKLPDDFTGCVIAWNSNNLDSKVGGNYNYLYRLFRKSNLKPFERD
ISTEIYQAGSTPCNGVEGFNCYFPLQSYGFQPTYGVGYQPYRVVVLSFE
LLHAPATVCGPKKSTNLVKNK
or
a variant thereof;
QPTESIVRFPNITNLCPFGEVFNATRFASVYAWNRKRISNCVADYSVLY
NSASFSTFKCYGVSPTKLNDLCFTNVYADSFVIRGDEVRQIAPGQTGNI
ADYNYKLPDDFTGCVIAWNSNNLDSKVGGNYNYLYRLFRKSNLKPFERD
ISTEIYQAGSTPCNGVKGFNCYFPLQSYGFQPTYGVGYQPYRVVVLSFE
LLHAPATVCGPKKSTNLVKNK
or
a variant thereof;
QPTESIVRFPNITNLCPFGEVFNATRFASVYAWNRKRISNCVADYSVLY
NSASFSTFKCYGVSPTKLNDLCFTNVYADSFVIRGDEVRQIAPGQTGTI
ADYNYKLPDDFTGCVIAWNSNNLDSKVGGNYNYLYRLFRKSNLKPFERD
ISTEIYQAGSTPCNGVKGFNCYFPLQSYGFQPTYGVGYQPYRVVVLSFE
LLHAPATVCGPKKSTNLVKNK
or
a variant thereof;
QPTESIVRFPNITNLCPFGEVFNATRFASVYAWNRKRISNCVADYSVLY
NSASFSTFKCYGVSPTKLNDLCFTNVYADSFVIRGDEVRQIAPGQTGKI
ADYNYKLPDDFTGCVIAWNSNNLDSKVGGNYNYRYRLFRKSNLKPFERD
ISTEIYQAGSKPCNGVEGENCYFPLQSYGFQPTNGVGYQPYRVVVLSFE
LLHAPATVCGPKKSTNLVKNK;
or
a variant thereof;
QPTESIVRFPNITNLCPFGEVFNATRFASVYAWNRKRISNCVADYSVLY
NSASFSTFKCYGVSPTKLNDLCFTNVYADSFVIRGDEVRQIAPGQTGKI
ADYNYKLPDDFTGCVIAWNSNNLDSKVGGNYNYLYRLFRKSNLKPFERD
ISTEIYQAGSTPCNGVKGFNCYFPLQSYGFQPTNGVGYQPYRVVVLSFE
LLHAPATVCGPKKSTNLVKNK
or
a variant thereof;
QPTESIVRFPNITNLCPFGEVFNATRFASVYAWNRKRISNCVADYSVLY
NSASFSTFKCYGVSPTKLNDLCFTNVYADSFVIRGDEVRQIAPGQTGKI
ADYNYKLPDDFTGCVIAWNSNNLDSKVGGNYNYRYRLFRKSNLKPFERD
ISTEIYQAGSTPCNGVQGFNCYFPLQSYGFQPTNGVGYQPYRVVVLSFE
LLHAPATVCGPKKSTNLVKNK;
or
a variant thereof;
QPTESIVRFPNITNLCPFGEVFNATRFASVYAWNRKRISNCVADYSVLY
NSASFSTFKCYGVSPTKLNDLCFTNVYADSFVIRGDEVRQIAPGQTGKI
ADYNYKLPDDFTGCVIAWNSNNLDSKVGGNYNYQYRLFRKSNLKPFERD
ISTEIYQAGSTPCNGVEGFNCYSPLQSYGFQPTNGVGYQPYRVVVLSFE
LLHAPATVCGPKKSTNLVKNK;
or
a variant thereof;
QPTESIVRFPNITNLCPFGEVFNATRFASVYAWNRKRISNCVADYSVLY
NSASFSTFKCYGVSPTKLNDLCFTNVYADSFVIRGDEVRQIAPGQTGNI
ADYNYKLPDDFTGCVIAWNSNNLDSKVGGNYNYRYRLFRKSNLKPFERD
ISTEIYQAGSKPCNGVKGFNCYFPLQSYGFQPTYGVGYQPYRVVVLSFE
LLHAPATVCGPKKSTNLVKNK;
or
a variant thereof;
QPTESIVRFPNITNLCPFGEVFNATRFASVYAWNRKRISNCVADYSVLY
NSASFSTFKCYGVSPTKLNDLCFTNVYADSFVIRGDEVRQIAPGQTGNI
ADYNYKLPDDFTGCVIAWNSNNLDSKVGGNYNYRYRLFRKSNLKPFERD
ISTEIYQAGNTPCNGVKGFNCYFPLQSYGFQPTYGVGYQPYRVVVLSFE
LLHAPATVCGPKKSTNLVKNK;
or
a variant thereof;
QPTESIVRFPNITNLCPFDEVFNATRFASVYAWNRKRISNCVADYSVLY
NLAPFFTFKCYGVSPTKLNDLCFTNVYADSFVIRGDEVRQIAPGQTGNI
ADYNYKLPDDFTGCVIAWNSNKLDSKVSGNYNYLYRLFRKSNLKPFERD
ISTEIYQAGNKPCNGVAGFNCYFPLRSYSFRPTYGVGHQPYRVVVLSFE
LLHAPATVCGPKKSTNLVKNK;
or
a variant thereof;
and
QPTESIVRFPNITNLCPFDEVFNATRFASVYAWNRKRISNCVADYSVLY
NFAPFFAFKCYGVSPTKLNDLCFTNVYADSFVIRGNEVSQIAPGQTGNI
ADYNYKLPDDFTGCVIAWNSNKLDSKVGGNYNYLYRLFRKSNLKPFERD
ISTEIYQAGNKPCNGVAGFNCYFPLRSYGFRPTYGVGHQPYRVVVLSFE
LLHAPATVCGPKKSTNLVKNK;
or
a variant thereof.

12. A chimeric polypeptide according to any one of claims 1 to 11 wherein the amino acid sequence encoding the one or more immunogenic epitope is a receptor binding domain of the first betacoronavirus spike protein comprising one or more amino acid variant of a second betacoronavirus spike protein.

13. A chimeric polypeptide according to claim 12 wherein the one or more amino acid variant is selected from an amino acid insertion, deletion or an amino acid substitution.

14. A chimeric polypeptide according to claim 13 wherein the second betacoronavirus spike protein is selected from the group consisting of a B.1.1.7 and Q (Alpha), B.1.351 (Beta), B.1.617.2 and AY (Delta), P.1 (Gamma), B.1.427 (Epsilon), B.1.429 (Epsilon), B.1.525 (Eta), B.1.526 (Iota) B.1.617.1 (Kappa), B.1.617.3, C.37 (Lambda), B.1.621 (Mu), B.1.1.529 (Omicron), and P.2 (Zeta) spike protein.

15. A chimeric polypeptide according to any one of claims 1 to 7 wherein the amino acid sequence encoding a trimerization domain is GYIPEAPRDGQAYVRKDGEWVLLSTFL or a variant thereof.

16. A chimeric polypeptide according to any one of claims 1 to 15, wherein the chimeric polypeptide further comprises an amino acid sequence encoding a purification tag.

17. A nucleic acid encoding a chimeric polypeptide according to any one of claims 1 to 16.

18. A nucleic acid according to claim 17, wherein the C terminal region of the first coronavirus spike protein further encodes the transmembrane domain and cytoplasmic tail of the first betacoronavirus spike protein.

19. A nucleic acid according to claim 18 wherein the nucleic acid is mRNA or DNA.

20. A vector comprising the nucleic acid of claim 18 or claim 19.

21. A host cell comprising the nucleic acid of claim 20.

22. A vaccine composition comprising a chimeric polypeptide according to any one of claims 1 to 16 or a trimer thereof, or a nucleic acid according to any one of claims 17 to 19, and a pharmaceutically acceptable carrier.

23. A method for prophylaxis or treatment of a betacoronavirus infection in a subject comprising administering an effective amount of a vaccine composition according to claim 22.

24. A use of a chimeric polypeptide according to any one of claims 1 to 12 or a trimer thereof, or a nucleic acid according to any one of claims 13 to 15 in the manufacture of a medicament for prophylaxis or treatment of SARS-COV-2 infection in a subject.
